(19) United States
(12) Patent Application Publication
(10) Pub. No.: US 2025/0388597 A1
(43) Pub. Date: Dec. 25, 2025

(54) BIFUNCTIONAL PHOTOCROSSLINKING PROBES FOR COVALENT CAPTURE OF PROTEIN-NUCLEIC ACID COMPLEXES IN CELLS

(71) Applicant: UNIVERSITY OF SOUTHERN CALIFORNIA, Los Angeles, CA (US)

(72) Inventors: Lin CHEN, Los Angeles, CA (US); Yi KOU, Los Angeles, CA (US)

(73) Assignee: UNIVERSITY OF SOUTHERN CALIFORNIA, Los Angeles, CA (US)

(21) Appl. No.: 18/881,379

(22) PCT Filed: Jul. 14, 2023
(86) PCT No.: PCT/US2023/070212

§ 371 Date: Jan. 6, 2025

Related U.S. Application Data
(60) Provisional application No. 63/389,580, filed on Jul. 15, 2022.

(51) Int. Cl. C07D 493/04; G01N 33/53

(52) U.S. Cl. CPC C07D 493/04; G01N 33/5308

(57) ABSTRACT

A new class of molecular probes is provided for real, efficient, stable, and selective capture of protein-nucleic acid complexes inside cells. The molecular probes have a nucleic acid-binding functional group and a photo-reactive diazirine based functional group, separated by a linker of a selected length or with a multi-arm core, thereby generating a photocrosslink between nucleic acids and proteins in close proximity. This is useful in various chromatin research, including the study of interactions between transcription factors and DNA.

### CROSS-REFERENCE TO RELATED APPLICATIONS

This application claims the benefit of priority under 35 U.S.C. § 119(e) to U.S. Provisional Patent Application No. 63/389,580 filed Jul. 15, 2022, the contents of which is incorporated herein by reference in its entirety.

### REFERENCE TO SEQUENCE LISTING

This application contains a sequence listing submitted as an electronic xml file named, “065715-000130 WOPT” created on Jul. 11, 2023 and having a size in bytes of 13,066 bytes. The information contained in this electronic file is hereby incorporated by reference in its entirety.

### FIELD OF INVENTION

This invention relates to functional small molecules for use in proximity ligation to identify and/or label protein-nucleic acid complexes.

### BACKGROUND

Protein-nucleic acids interactions are fundamental to a wide range of cellular processes, from genomic DNA replication, repair and transcription to RNA processing, translation and regulation. Nucleic acids such as cytoplasmic DNA and viral RNA also regulate cellular signaling pathways involved in immune responses, aging and diverse human disease. A major challenge in studying protein-nucleic acids interactions in situ is the capturing and isolation of protein-nucleic acid complexes inside cells, as most of these non-covalent complexes are dynamic and dissociate during the isolation process. As part of the effort to address this question, a variety of techniques have been developed to capture protein-nucleic acid complexes in cells, including direct UVC (254 nm) crosslinking between RNA and RNA binding proteins or using UVA (365 nm) with RNA metabolically labelled with 4-thio-uridine (4SU) or 6-thio-guanosine (6SG). While these methods have greatly facilitated the study of protein/RNA interactions, the low crosslinking efficiency, the use of short wavelength UVC (which damages protein, DNA and RNA), or the need to use metabolic labelling with 4SU and 6SG, are still major limitations.

In the study of the interactions between transcription factors (TFs) and DNA, chromatin immuno-precipitation followed by sequencing (ChIP-Seq), has widely been used. However, major limitations of the current approach have been increasingly recognized, especially for low abundance TFs; and in traditional protocols, the cell fixation step by use of formaldehyde could be a major contributor to data irreproducibility, because it negatively impacts the activity of antibodies and other aspects of the ChIP-based experiments (e.g. ChIP-seq, Hi-C or HiChIP). A substantial number of replicate ChIP-seq datasets in the ENCODE database have low correlation (r˜0.5-0.6). Another major issue is that a large fraction (45-80%) of detected DNA sequences lack the expected binding motif, raising the question of whether the DNA fragments are associated with the TF target via indirect mechanisms or simply due to non-specific trapping (see FIG. 1A). These limitations severely undermine the effectiveness of ChIP-seq in mechanistic studies, such as analyzing the functional impact of genetic variations in TF binding sites. Part of the problem has been attributed to the instability and limited quality of antibodies, especially for transcription factors. It has also become increasingly clear that another step of the protocol, namely cell fixation by formaldehyde, could be a major contributor to the data irreproducibility of ChIP-based experiments.

The wide range usage of formaldehyde as a crosslinker in the aforementioned technologies is based on the long-held but poorly understood belief that it can crosslink protein to DNA. Formaldehyde can crosslink two primary amine groups through a Schiff base intermediate to form a methylene bridge between two spatially proximal lysine residues. The reaction is highly facile, which explains the high efficiency of formaldehyde-based cross-linking of protein complexes. The crosslinking between protein and DNA, however, is an entirely different story. The exocyclic amino groups from the nucleic acids are inefficient nucleophiles due to delocalized conjugation with aromatic ring systems of the nucleoside bases (FIG. 1E). Although crosslinking products between certain amino acids and short oligonucleotides were observed by mass spectrometry under extreme conditions, the yield is very low and the crosslinked products are unstable. These observations raise the question of whether formaldehyde could directly crosslink protein and DNA as a stable complex at all. Earlier studies have shown that formaldehyde failed to crosslink purified transcription/DNA complexes in vitro despite its efficient in vivo crosslinking of higher-order chromatin complexes. On the other hand, certain transcription factors, such as NF-kB, STAT3, and the fly insulator factor Elba, which form ring-like structures or higher-order complexes to wrap around DNA, could be stably cross-linked to DNA when a secondary protein crosslinker such as disuccinimidyl suberate (DSS) was used. These observations hint that the apparent success of formaldehyde-based crosslinking of protein-DNA complexes in cells is unlikely due to the direct crosslinking reaction between the two, but rather through the crosslinking of protein complexes that trap the DNA. This mechanism of DNA trapping by crosslinked protein complexes is likely the major source of signal noises of current CHIP-seq data (FIG. 1A). Furthermore, the empirical and poorly characterized step of formaldehyde crosslinking can cause a number of additional problems as described in the literature.

First, direct modification of transcription factors by formaldehyde at the DNA binding face, which is often enriched with lysine residues, could lead to failed capture of the protein-DNA complex (see FIG. 5G), especially for TFs that bind DNA highly dynamically, or could even cause artifact. Second, formaldehyde crosslinking could impact the activity of antibodies used to capture TFs, either directly by modifying residues in the epitope or indirectly by locking the protein structure rendering the epitope inaccessible. As a result, varying cell fixation conditions (formaldehyde concentration and reaction time) could exacerbate the variability of antibody reactivity. These problems are potentially more significant in the study of low abundant TFs (vs histone proteins) where a high concentration of formaldehyde fixation may be required to capture sufficient protein/DNA complexes. Overall, the highly reactive but non-specific modifications to proteins and ineffective crosslinking to DNA by formaldehyde is a major limitation in the current ChIP-based technologies.

Various reports from different labs have realized that formaldehyde cannot crosslink certain proteins to nucleic acids even if they are in close proximity inside the nucleus. The main reason behind this lies in the crosslinking chemistry that formaldehyde requires: an active nucleophilic attack from one side of the crosslinking target onto the carbon atom in the Schiff base imine that it has already formed by crosslinking from the other side of the target. Although amine groups are a nucleophile in general, those exocyclic amine groups from nucleic acids (for example DNA) are almost in an inactive state, as the electrons of the nitrogen on it are somewhat restricted by a delocalized conjugation from the nearby aromatic base ring systems. This makes the nucleic acids innately an inactive nucleophile to be directly crosslinked to protein via formaldehyde mediated crosslinking (FIG. 1E) in these ChIP based methods. Instead the specific genomic loci information extracted from these technologies are somewhat the rough estimation/simulation from nearby protein-protein (such as histone-transcription factor, because histone binds DNA extensively) crosslink result. Therefore, the direct modification of transcription factors by formaldehyde at the DNA binding face would lead to failed capture of the protein-DNA complex, especially for transcription factors that bind DNA highly dynamically. Obtaining these results by formaldehyde are thus deviated from the actuality and could even cause unnecessary artifact since it is histone crosslinked to nearby proteins rather than the DNA itself. Secondly, because there are a large number of proteins inside the cell nucleus, the good crosslinking ability for formaldehyde towards these proteins often results in high and undesired noises despite of antibody extraction, since the desired target for the antibody is often crosslinked to multiple other proteins nearby. And because of such abundance of protein to DNA in the nucleus, more often than not, these protein-protein crosslinking noises are so high that some regions on the chromosomes are forbidden areas for formaldehyde mediated ChIP (FIG. 1E). Thirdly, since the regio-physical and chemical features of various proteins inside the cell nucleus are very heterogeneous, thus the different formaldehyde reactivity toward distinctive proteins, protein complexes, and various subcellular regions and structures are already considered to be the source of a number of problems encountered in formaldehyde-based protocols. This often brings the result that certain proteins or proteins complexes may be more or less crosslinked by formaldehyde (e.g., due to different amounts and structural distribution of the surface lysine residues), rendering certain genome regions over- or under-sampled in ChIP-based assays (FIG. 1E). Finally, formaldehyde crosslinking could impact the activity of antibodies used to capture TFs, either directly by modifying residues in the epitope or indirectly by locking protein structure rendering the epitope inaccessible (FIG. 1E). Thus, although ChIP based methods can have certain repeatable results, the real and precise protein-nucleic acids crosslinking remains a challenge for formaldehyde typed crosslinkers.

In addition to formaldehyde, UV irradiation (e.g., short-wavelength UVC) has also been used to crosslink protein to RNA and DNA. UV crosslinking can yield stable covalent products that can be digested by proteases and nucleases to generate peptide/oligonucleotide conjugates for subsequent mass spectrometry analyses (XL-MS). While this represents a promising method for mapping protein-nucleic acid interactions in vitro and in cells, a major disadvantage of these approaches is the short wavelength UVC (˜250 nm) required to induce cross linking between natural protein and nucleic acids and the low crosslinking efficiency. A short wavelength (E=hc/wavelength, where hc is constant) brings high UV dosage (continuous or pulsed UV laser) and to increase crosslinking yield under such condition would instead lead to broad UV damage to both proteins and DNA or RNA.

Given the significant drawbacks of formaldehyde and UV crosslinking discussed above, it remains challenging to continue using crosslinker of such type.

Therefore, it is an objective of the present invention to provide new compounds and systems for use in capturing protein-DNA complexes in cells with high efficiency, selectivity (i.e., only targeting DNA-bound proteins), and stability (to enable robust isolation of protein-DNA complexes for subsequent analyses by DNA sequencing or protein mass spectrometry).

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

In various embodiments, the present invention provides a compound of Formula (I): A-L1-(C)ₙ-((L2)ₙ-B)ₘ Formula (I), wherein: A represents a nucleic acid-binding functional group derived from psoralen, methyltrioxsalen, benzophenone, 4′,6-diamidino-2-phenylindole (DAPI), a Hoechst dye, a polyamide, or a G quartet binding molecule, kethoxal, or a derivative thereof; L1 is absent or represents a first linker, C represents, when n=1, a core moiety having at least two functional groups each separately for attachment to L1 and attachment to at least one arm represented by L2-B; or C is absent when n=0; L2 represents, when n=1, independently a second linker for each arm represented by L2-B; or L2 is absent when n=0; B represents independently for said each arm: a photo-reactive functional group comprising diazirine or its derivative or an aryl azide or its derivative, optionally the aryl azide or its derivative selected from phenyl azide, orthro-hydroxyphenyl azide, meta-hydroxyphenyl azide, tetrafluorophenyl azide, ortho-nitropenyl azide, meta-nitropenyl azide, or azdo-methylcoumarin; or a detectable functional group; wherein in at least one said each arm, B represents the photo-reactive functional group; n=0 or 1; m represents number of arms represented by (L2)ₙ-B, wherein m is an integer being 1 or greater when n=1, or m=1 when n=0.

In some embodiments, at least one of L1 and L2 is not absent, and the at least one of L1 and L2 is cleavable.

In some embodiments, L1, L2, or both independently comprise one or more of a sulfoxide-containing mass spectrometry (MS)-cleavable bond, an acid-cleavable C—S bond, a disulfide group, and an azo group.

In some embodiments, n=0, m=1, and the compound is represented by Formula (II): A-L1-B Formula (II), wherein L1 is absent or the first linker.

In some embodiments, A is an amine-containing or amine-reactive derivative of the psoralen, an amine-containing or amine-reactive derivative of the methyltrioxsalen, an amine-containing or amine-reactive derivative of the benzophenone, an amine-containing or amine-reactive derivative of the 4′,6-diamidino-2-phenylindole (DAPI), an amine-containing or amine-reactive derivative of the Hoechst dye, an amine-containing or amine-reactive derivative of the polyamide, or an amine-containing or amine-reactive derivative of the G quartet binding molecule, or an amine-containing or amine-reactive derivative of kethoxal, optionally A being derived from succinimidyl-[4-(psoralen-8-yloxy)]-butyrate (SPB) or 4′-aminomethyltrioxsalen (4AMT); B comprises a diazirine or a diazirine alkyne, optionally an amino diazirine alkyne (AAD); and L1 is absent or the first linker, wherein the first linker comprises one or more of (i) a cleavable bond, (ii) an oligomer or polymer having a repeating unit of —OCH₂CH₂—, and (iii) an unsaturated moiety, optionally selected from a carbon-carbon double bond, a carbon-carbon triple bond, or an aryl group.

In some embodiments, L1-B is derived from succinimidyl 6-(4,4′-azipentanamido)hexanoate (NHS-LC-SDA), succinimidyl 2-((4,4′-azipentanamido)ethyl)-1,3′dithiopropionate (NHS-SS-Diazirine), or 2-(3-(But-3-yn-1-yl)-3H-diazirin-3-yl)ethan-1-amine (AAD); and/or wherein A is derived from 4′-aminomethyltrioxsalen (4AMT) or succinimidyl-[4-(psoralen-8-yloxy)]-butyrate (SPB); and wherein optionally the photocrosslinking molecule is represented by Formula (IIa) or Formula (IIc):

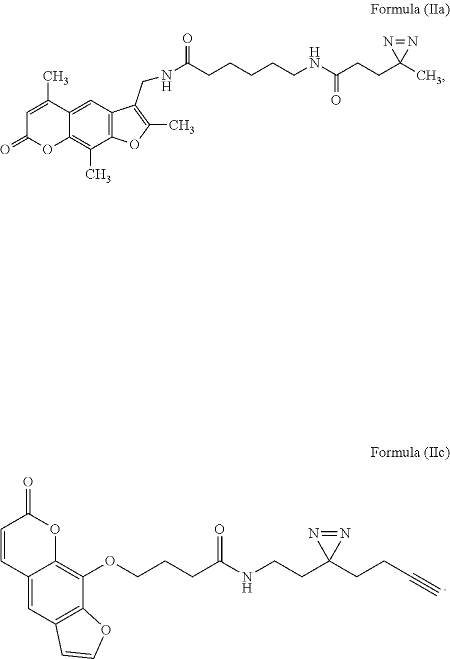 [SMILES: Cc1oc2c(C)c3oc(=O)cc(C)c3cc2c1CNC(=O)CCCCCNC(=O)CCC1(C)N=N1 ; C#CCCC1(CCNC(=O)CCCOc2c3occc3cc3ccc(=O)oc23)N=N1]

In some embodiments, A is derived from succinimidyl-[4-(psoralen-8-yloxy)]-butyrate (SPB) or 4′-aminomethyltrioxsalen (4AMT); B comprises a diazirine or a diazirine alkyne, optionally an amino diazirine alkyne (AAD); and L1 is the first linker comprising one or more of (i) a cleavable bond, (ii) an oligomer or polymer having a repeating unit of —OCH₂CH₂—, and/or (iii) an unsaturated moiety, said unsaturated moiety optionally selected from a carbon-carbon double bond or an aryl group; and wherein optionally the photocrosslinking molecule is represented by Formula (IIb), Formula (IId), Formula (IIe), or Formula (IIf):

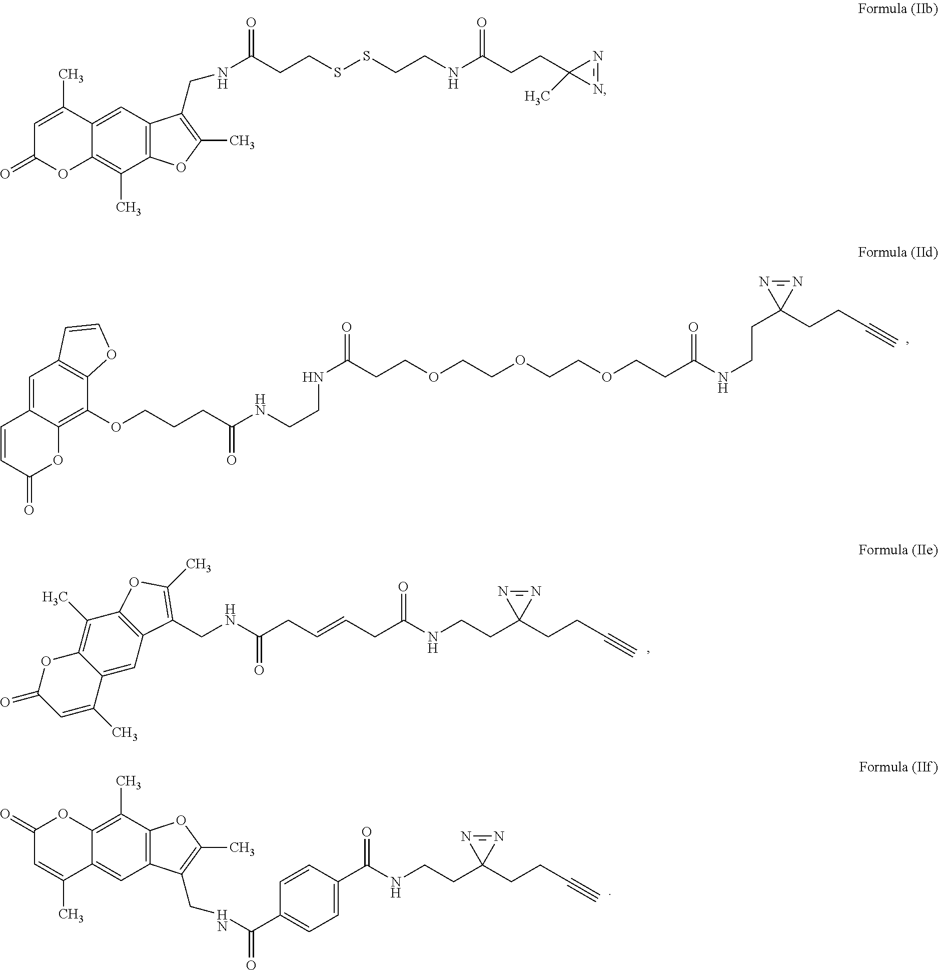

In some embodiments, A is selected from the group consisting of:

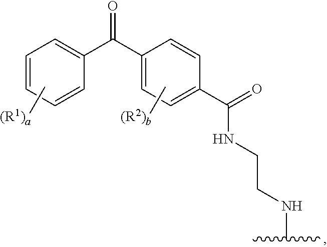

wherein:
R¹ is independently H, halo, OH, optionally substituted alkoxy, or optionally substituted alkyl;
R² is independently H, halo, OH, optionally substituted alkoxy, or optionally substituted alkyl;
a is 0, 1, 2, 3, 4, or 5; and
b is 0, 1, 2, 3, or 4;

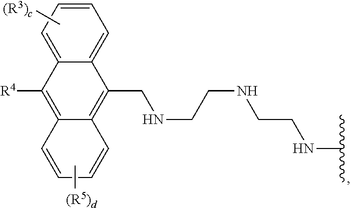

wherein:
R³ is independently H, halo, OH, optionally substituted alkoxy, or optionally substituted alkyl;
R⁴ is H, halo, OH, optionally substituted alkoxy, or optionally substituted alkyl;
R⁵ is independently H, halo, OH, optionally substituted alkoxy, or optionally substituted alkyl;
c is 0, 1, 2, 3, or 4; and
d is 0, 1, 2, 3, or 4;

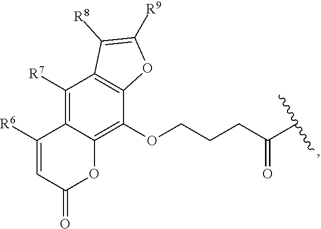

wherein:
R⁶ is H, halo, OH, optionally substituted alkoxy, or optionally substituted alkyl;
R⁷ is H, halo, OH, optionally substituted alkoxy, or optionally substituted alkyl;
R⁸ is H, halo, OH, optionally substituted alkoxy, or optionally substituted alkyl; and
R⁹ is H, halo, OH, optionally substituted alkoxy, or optionally substituted alkyl;

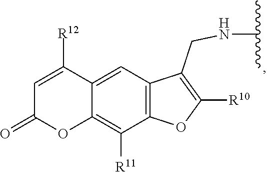

wherein:
R¹⁰ is H, halo, OH, optionally substituted alkoxy, or optionally substituted alkyl;
R¹¹ is H, halo, OH, optionally substituted alkoxy, or optionally substituted alkyl; and
R¹² is H, halo, OH, optionally substituted alkoxy, or optionally substituted alkyl;

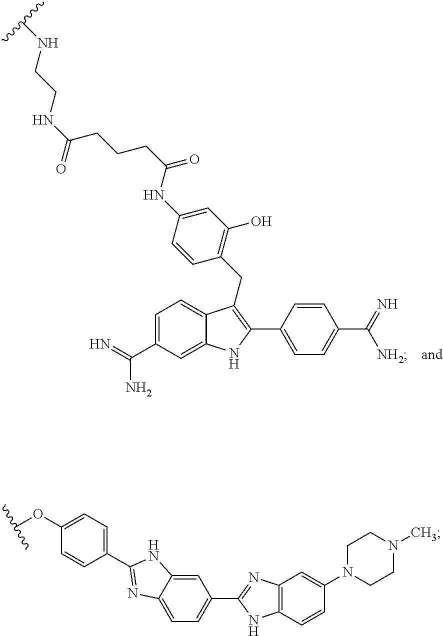 [SMILES: *NCCNC(=O)CCCC(=O)Nc1ccc(Cc2c(-c3ccc(C(=N)N)cc3)[nH]c3cc(C(=N)N)ccc23)c(O)c1 ; *Oc1ccc(-c2nc3ccc(-c4nc5cc(N6CCN(C)CC6)ccc5[nH]4)cc3[nH]2)cc1]

L1 is absent or L is selected from the group consisting of:

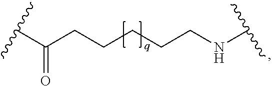

wherein:
q is 0, 1, 2, 3, or 4;

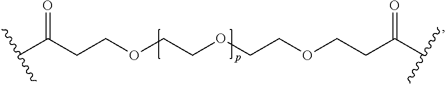

wherein:
p is 0, 1, 2, 3, or 4;

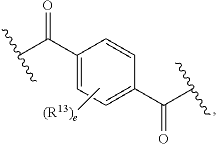

wherein:
R¹³ is independently H, halo, OH, optionally substituted alkoxy, or optionally substituted alkyl;
and e is 0, 1, 2, 3 or 4;

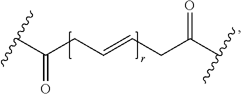

wherein:
r is 0, 1, 2, 3, or 4;

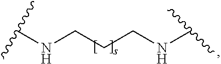

wherein:
s is 0, 1, 2, 3, or 4;

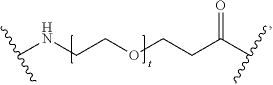

wherein:
t is 0, 1, 2, 3 or 4;

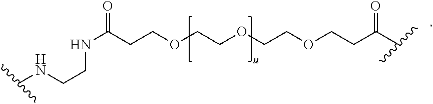

u is 0, 1, 2, 3, or 4;

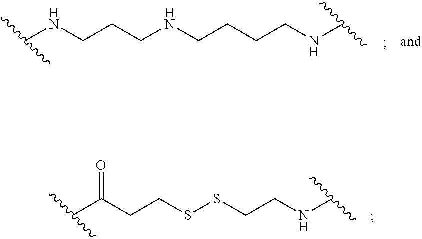 [SMILES: *NCCCCNCCCN* ; *NCCSSCCC(*)=O]

and
B is selected from the group consisting of:

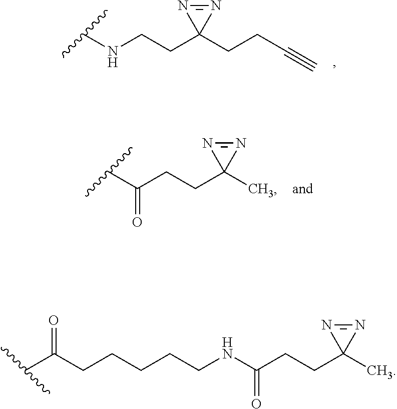 [SMILES: *NCCC1(CCC#C)N=N1 ; *C(=O)CCC1(C)N=N1 ; *C(=O)CCCCCNC(=O)CCC1(C)N=N1]

In some embodiments, A is selected from the group consisting of:

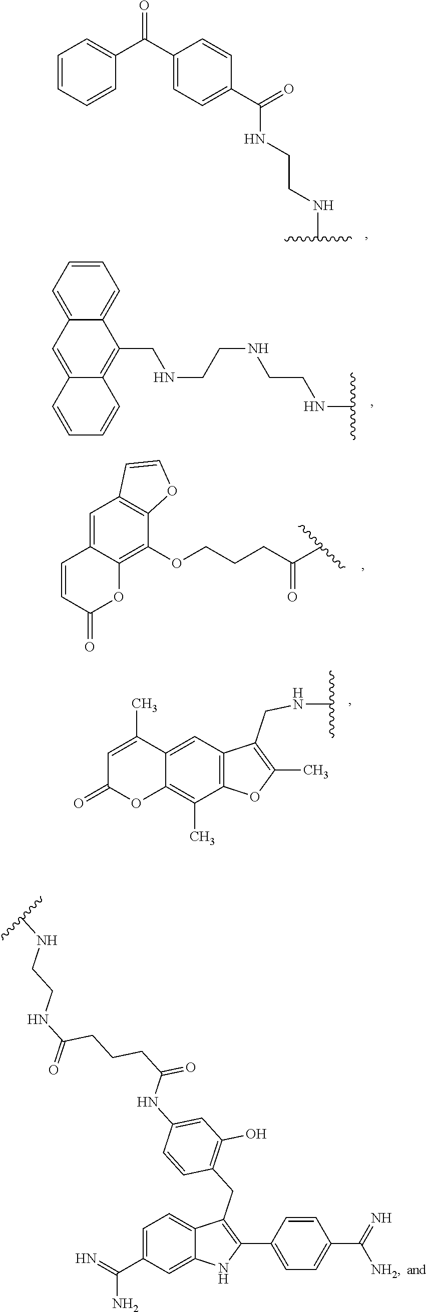

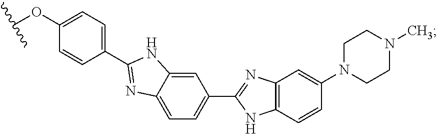 [SMILES: *Oc1ccc(-c2nc3ccc(-c4nc5cc(N6CCN(C)CC6)ccc5[nH]4)cc3[nH]2)cc1]

L1 is absent or L1 is selected from the group consisting of:

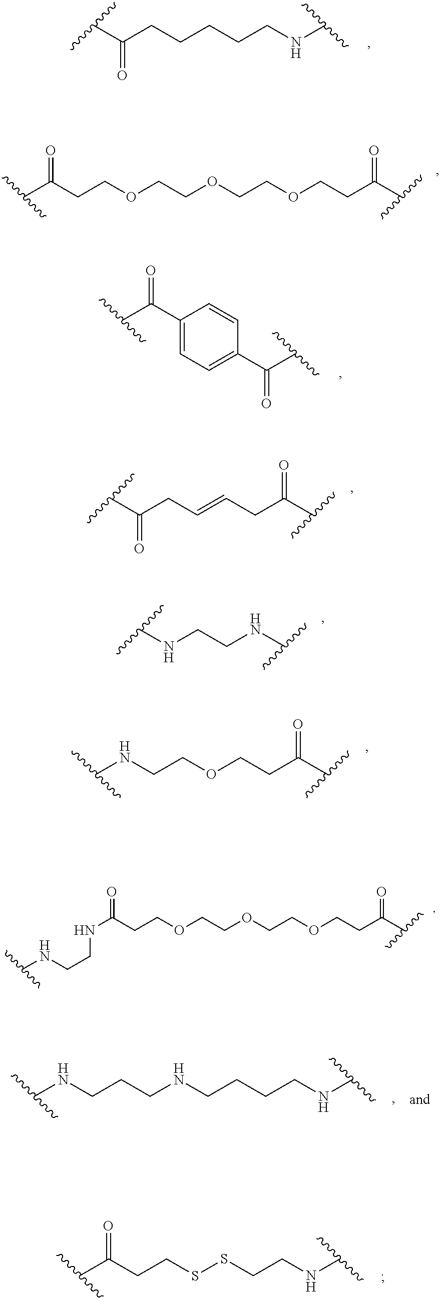

and
B is selected from the group consisting of:

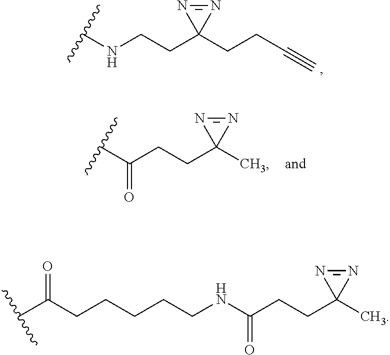 [SMILES: *NCCC1(CCC#C)N=N1 ; *C(=O)CCC1(C)N=N1 ; *C(=O)CCCCCNC(=O)CCC1(C)N=N1]

In some embodiments, the compound is:

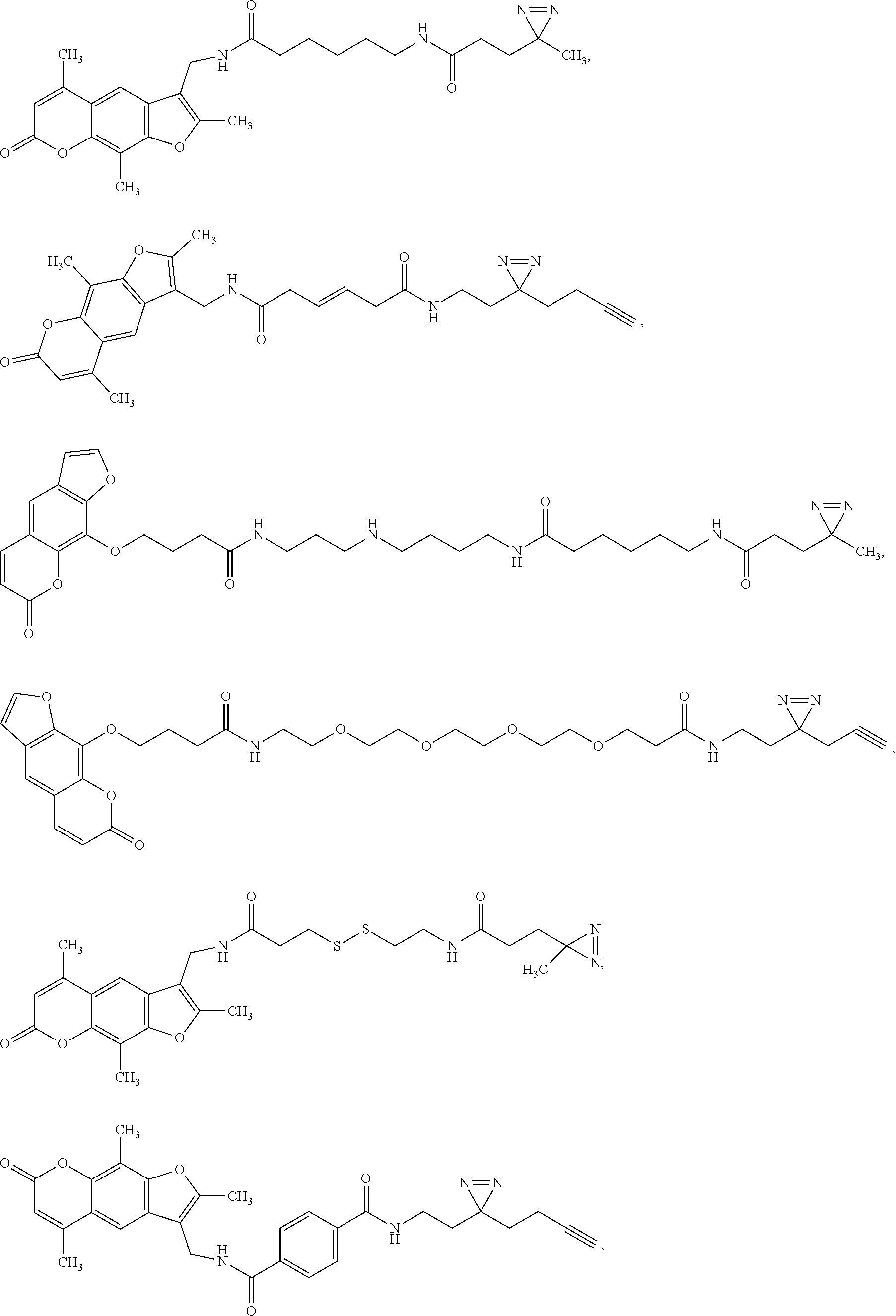 [SMILES: Cc1oc2c(C)c3oc(=O)cc(C)c3cc2c1CNC(=O)CCCCCNC(=O)CCC1(C)N=N1 ; C#CCCC1(CCNC(=O)C/C=C/CC(=O)NCc2c(C)oc3c(C)c4oc(=O)cc(C)c4cc23)N=N1 ; CC1(CCC(=O)NCCCCCC(=O)NCCCCNCCCNC(=O)CCCOc2c3occc3cc3ccc(=O)oc23)N=N1 ; C#CCC1(CCNC(=O)CCOCCOCCOCCOCCNC(=O)CCCOc2c3occc3cc3ccc(=O)oc23)N=N1 ; Cc1oc2c(C)c3oc(=O)cc(C)c3cc2c1CNC(=O)CCSSCCNC(=O)CCC1(C)N=N1 ; C#CCCC1(CCNC(=O)c2ccc(C(=O)NCc3c(C)oc4c(C)c5oc(=O)cc(C)c5cc34)cc2)N=N1]

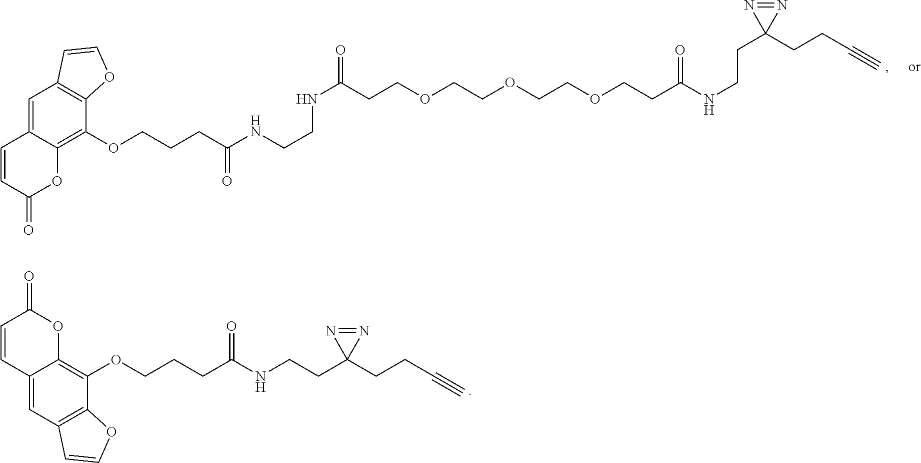 [SMILES: C#CCCC1(CCNC(=O)CCOCCOCCOCCC(=O)NCCNC(=O)CCCOc2c3occc3cc3ccc(=O)oc23)N=N1 ; C#CCCC1(CCNC(=O)CCCOc2c3occc3cc3ccc(=O)oc23)N=N1]

In some embodiments, L1 comprises 2 to 20 carbons or 20-100 carbons in length.

In some embodiments, n=1, m is an integer being 2 or greater, and C represents a core moiety having at least three functional groups each separately for attachment to L1 and attachment to the at least two arms each represented by (L2-B), so that the compound is represented by Formula (III):

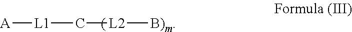

In some embodiments, B comprises diazirine or an azide diazirine in one of the at least two arms, and B represents a detectable functional group in another one of the at least two arms, said detectable function group comprising a fluorophore, a biotin, a chromophore, a chromogen, a quantum dot, a fluorescent microsphere, or a nanoparticle. In some embodiments, L1, L2, or both independently comprise one or more of (i) a cleavable bond, (ii) an oligomer or polymer having a repeating unit of —OCH₂CH₂—, and (iii) an unsaturated moiety. In some embodiments, C represents a dendritic core moiety comprising at least three surface functional groups each separately for attachment to L1 and attachment to the at least two arms each represented by L2-B. In some embodiments, L1, L2, or both independently comprise a triazole in bonding with A.

In various embodiments the present invention provides a method of crosslinking a nucleic acid with a protein in a system, comprising: providing a compound of the present invention; providing a system, wherein the system comprises a nucleic acid and a protein; contacting the compound with the system; and irradiating the system and the compound with an ultraviolet light under conditions effective to crosslink the nucleic acid with the protein. In some embodiments, the system is a live cell. In some embodiments, the ultraviolet light is between 300 nm and 370 nm in wavelength. In some embodiments, the method further comprising performing one or more of immuno precipitation, DNA and RNA complexes extraction using organic solvents, chromatographic separation, chromatin precipitation, 3D chromatin conformation capture, mass spectrometry, and electrophoresis, with the system. In some embodiments, element L1, L2, or both of the compound is independently cleavable, and the method further comprises adding a cleaving agent to the system to cleave the elements L1, L2, or both; or wherein element A of the compound is derived from psoralen, and the method further comprises applying an ultraviolet light of about 230 nm in wavelength to cleave the element A; thereby generating a fingerprint of crosslinked proteins in proximity to nucleic acids in the system.

In some embodiments, the present invention provides a method for preparing a compound of Formula (III), the method comprising: providing an azide derivative of a nucleic acid-binding, photo-reactive agent comprising psoralen, methyltrioxsalen, benzophenone, 4′,6-diamidino-2-phenylindole (DAPI), a Hoechst dye, a polyamide, or a G quartet binding molecule, kethoxal, or a derivative thereof; providing an azide derivative of a photo-reactive agent that comprises a diazirine moiety so as to obtain an azide-diazirine bifunctional, photo-reactive agent, and said photo-reactive agent optionally further comprising an alkyne group, or providing an aryl azide, said aryl azide optionally selected from phenyl azide, orthro-hydroxyphenyl azide, meta-hydroxyphenyl azide, tetrafluorophenyl azide, ortho-nitropenyl azide, meta-nitropenyl azide or azdo-methylcoumarin; optionally providing an azide derivative of a detectable agent comprising a fluorophore, a biotin, a chromophore, a chromogen, a quantum dot, a fluorescent microsphere, or a nanoparticle; providing a multi-arm agent having at least three functional groups each independently comprising an alkyne; and combining each azide derivatives and, if provided, the aryl azide, together with the multi-arm agent in one reaction vessel to prepare the compound.

In some embodiments, the multi-arm agent has at least three functional groups each independently comprising a cyclooctyne group. In some embodiments, the nucleic acid-binding, photo-reactive agent comprises a first primary amine functional group, and providing the azide derivative of the nucleic acid-binding, photo-reactive agent comprises converting the first primary amine functional group to a first azide-containing moiety, optionally via reacting the nucleic acid-binding, photo-reactive agent with imidazole-1-sulfonyl azide; and/or wherein the photo-reactive agent that comprises a diazirine moiety further comprises a second primary amine functional group or is modified with the second primary amino functional group, and providing the azide derivative of said photo-reactive agent comprises converting the second primary amine functional group to a second azide-containing moiety, optionally via reacting said photo-reactive agent with imidazole-1-sulfonyl azide.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A-FIG. 1C depicts that formaldehyde crosslinking of proteins in the nucleus trap large scale protein-DNA complexes with many DNA and proteins associated together. (FIG. 1A) Instead of capturing targeted transcription factor (TF, blue oval) by a selected antibody (Ab, inverse Y shape) and its binding site (TFBS, blue bar); (FIG. 1B) the antibody pull down drag down a lot of other proteins (various colors and shapes) and non-specific sites (NS, yellow bars); (FIG. 1C) the ChIP-seq track contains a high percentage of false signals.

FIG. 1D depicts a general design of the BFPX strategy of the invention herein.

FIG. 1E describes in molecular details as why formaldehyde is an undesired crosslinker towards protein-nucleic acids crosslinking. The main reason is because the amine from nucleic acids such as DNA is not an active nucleophile, thus making the crucial attacking onto the imine intermediate in low yield and resulting in failed or inaccurate capture or crosslink. Other disadvantages include high noise from other proteins in the nucleus, different protein crosslinking effects, histone mediated inaccuracy or artifact, protein epitope masking or distortion.

FIG. 2A-FIG. 2C are schematic diagrams of an exemplary probe design. The probe is ‘bi-functional’ because on one side it confers the nucleic acids with the real free primary amine group by photo-reactive intercalator psoralen, and through this amine linkage on the other side we have equipped it with a same spectrum photo-reactive crosslinker diazirine group, which can crosslink a nearby biomolecule under photo-activation to form a carbene. FIG. 2A depicts an example of psoralen-based BFPX probes wherein the DNA binding/crosslinking head is a psoralen derivative (4′-aminomethyltrioxsalen, 4AMT) and the protein crosslinking head is a diazirine group. The dashed box is the linker region that can be synthetically engineered to have variable length, cleavability, or functional groups (represented by R) for the enrichment and/or fluorescent labelling of the crosslinked Protein-DNA complexes. FIG. 2B depicts a schematic diagram of an exemplary probe design. FIG. 2C depicts a schematic diagram of an exemplary probe design.

FIG. 3A and FIG. 3B depict exemplary approaches to expansion of bifunctional probes through multi-arm PEG DBCO cooper-free clickable core to obtain multifunctional probes. The PEG DBCO clickable arms can be increased to more than 4 shown here. Multiple protein photo-crosslinkable ends can also be clicked into the core for use in capturing multiple nearby proteins. PEG arm length can be chosen. Nucleic acid-binding, photocrosslinkable ends are based on psoralen, benzophenone, DAPI, a polyamide, or a G-quartet binding molecule, wherein each is derived with an amine group; and via imidazole-1-sulfonyl azide, the amine-based nucleic acid-binding, photocrosslinkable ends are converted to azide-based (azido) nucleic-acid binding, photocrosslinkable ends. Azide-based compounds (e.g., azide-based nucleic-acid binding, photocrosslinkable ends; an azide fluorophore; azo biotin-azide; as well as azide-based protein photocrosslinkable ends) can all conjugate with a multi-arm core (e.g., PEG-DBCO) through copper-free click chemistry.

FIG. 3C depicts an exemplary synthetic route for using a cleavable linker—being cleavable due to collision-induced dissociation (CLD) during mass spectrometry—in forming an exemplary BFPX probe using DAPI molecule as the DNA binding head. Through these designed functions, the molecule not only binds to DNA double strand but also contains (1) a fluorescent locator; (2) a MS CID (collision induced dissociation) cleavable finger print that could facilitate MS analysis using the parts after dissociation, via azide-tagged, acid-cleavable disuccinimidyl bissulfoxide (Azide-A-DSBSO; Bis(2,5-dioxopyrrolidin-1-yl) 3,3′-((2-(3-azidopropyl)-2-methyl-1,3-dioxane-5,5-diyl)bis(methylenesulfonyl))dipropionate; a mass spectrometry-cleavable crosslinker for studying protein-protein interactions), to introduce a mass spectrometry-cleavable functional group (i.e., acid-cleavable C—S bonds); and also (3) a S—S(disulfide) cleavable extraction biotin handle, which eases the enrichment process by streptavidin beads. Azide-A-DSBSO possesses two N-hydroxysuccinimide (NHS) ester groups for targeting amines, a ˜14 Å spacer length, two symmetrical acid-cleavable C—S bonds, and a central bioorthogonal azide tag; and the post-cleavage spacer (after cleavage of the C—S bonds) yields tagged peptides for unambiguous identification by collision-induced dissociation in tandem MS.

FIG. 3D depicts exemplary unsaturated moieties as linkers and their exemplary synthetic routes in forming a BFPX probe.

FIG. 3E depicts a schematic of structure-based design of BFPX probes using DAPI or Hoechst 33258 as the DNA binding heads. Top panel: left, the crystal structure of DAPI bound to double stranded DNA, right, the DNA-binding face (orange shaded area) of DAPI should be avoided in synthetic modification; blue arrows indicate potential sites for introducing linkers bearing the protein capturing groups (e.g. the diazirine heads), green arrows indicate potential sites for introducing photo affinity labeling groups that crosslink to DNA because these position of DAPI are proximal to DNA. Bottom panel: left, the crystal structure of Hoechst 33258 bound to double stranded DNA, right, the DNA-binding face (orange shaded area) of Hoechst 33258 should be avoided in synthetic modification; blue arrows indicate potential sites for introducing linkers bearing the protein capturing groups (e.g. the diazirine heads), green arrows indicate potential sites for introducing photo affinity labeling groups that crosslink to DNA because these positions are proximal to DNA.

FIG. 4 shows chemical structures of four exemplary psoralen-based BFPX probes synthesized in preliminary studies in the Example section. Exemplary synthetic routes and mass spectrometry confirmation on the synthesized product for each of 4AMT-LC-SDA, 4AMT-SDAD (also denoted as 4AMT-LC-SDAD), and SPB-PEG3-AAD are shown in FIG. 5A-FIG. 5B, FIG. 6A-FIG. 6B, and FIG. 8A-FIG. 8B, respectively.

FIG. 5A and FIG. 5B depict probe synthesis and confirmation by mass spectrometry (MS). The probe has flexible linkage in between, and an example final probe molecule can be easily synthesized through the amine—N-hydroxysuccinimide esters (NHS) conjugation chemistry, e.g., reaction of 4′-aminomethyltrioxsalen (4-AMT) and NHS-LC-Diazirine (NHS-LC-SDA, succinimidyl 6-(4,4′-azipentanamido)hexanoate) in a one-step mild condition, with a relatively good yield estimated by mass spectrometry. In FIG. 5A, the green frame encircles the DNA binding head, and the red frame encircles the protein binding head. In FIG. 5B showing the MS spectra, a major peak with good yield from the reaction mixture showing the final compound in +Na⁺ (480+23) or double final compounds in +Na⁻ (960+23) mass of multiple reaction set ups (indicated by the arrow).

FIG. 5C-FIG. 5E depict gel electrophoresis results, confirming the success of probes from various signal channels (DNA, protein, larger pore size) in efficiently and selectively crosslinking an exemplary double-stranded DNA and a DNA-binding protein.

FIG. 5F depicts electrophoresis analysis of 4AMT-LC-SDA in a suspension with GM12878 cell lines with different sonication times, in comparison to formaldehyde and UV only in the suspension, demonstrating the overall expected efficiency of applying this probe in vivo.

FIG. 5G depicts a native EMSA assay of the effect of Formaldehyde (FA), BFPX probe (4AMT-LC-SDA), and UVA (365 nm) on the DNA binding by NFAT1. All binding reactions contain 10 μM 27 mer dsDNA (5′6-FAM labeled) with a NFAT-binding site, and various combinations of NFAT1 protein (14 μM), FA (1% v/v), 4AMT-LC-SDA (20 μM), with or without UV illumination (365 nm, LED, 30 Watt, sample distance 2 cm, exposure time 15 min at 4 degree C.). 10 μl of the binding reaction is run on a 10% PAGE 0.5×TBE gel. The same gel was visualized by FAM fluorescence for DNA (top) and by Coomassie blue stain for protein (bottom). The higher smearing bands in lane 3, 8, 9, and 10 are most likely non-specific protein complexes often seen when an excessive amount of protein is used to ensure all DNA is bound.

FIG. 5H depicts denaturing SDS gel analysis of covalent capture of in vitro assembled NFAT1/DNA complexes by the BFPX probe 4AMT-LC-SDA (denoted as “LC-SDA” for Lanes 1, 2, 3) and 4AMT-LC-SDAD (denoted as “LC-SDAD” for Lanes 4, 5, 6). All reactions contain 10 μM 27-mer dsDNA (5′6-FAM labeled) with a NFAT1-binding site, and 14 μM NFAT1, and 25 μM BFPX probe (4AMT-LC-SDA, lanes 1-3; or 4AMT-LC-SDAD, lanes 4-6). The probe binding and UV crosslinking were carried out as described in FIG. 5G and FIG. 7. The crosslinked complexes (lane 3 and lane 6) were further treated with DTT (100 mM, heat 50 degree C. for 30 min). The reaction mixtures were run on SDS gel and visualized by FAM fluorescence for DNA (top) and by Coomassie blue stain for protein (bottom) as described in FIG. 7.

FIG. 6A depicts the synthesis of cleavable BFPX probe, 4AMT-SDAD, which involves coupling between 4AMT and NHS-SS-diazirine.

FIG. 6B is an ESI mass spectrum confirming the synthesized 4AMT-SDAD.

FIG. 7 depicts denaturing SDS gel analysis of covalent capture of in vitro assembled MEF2/DNA and NFAT1/DNA complexes by the BFPX probe, SPB-AAD. Reactions of lanes 1-7 contain 10 μM 44-mer dsDNA (5′6-FAM labeled) with a MEF2-binding site, and 40 μM MEF2A; Reactions of lanes 8-12 contain 10 μM 27 mer dsDNA (5′6-FAM labeled) with a NFAT1-binding site, and 14 μM NFAT1; The protein/DNA complexes are incubated at room temperature for 30 min, and treated with various combination of BFPX probe (SPB-AAD) and UV illumination as described in FIG. 5G. The concentrations of SPB-AAD in lanes 3, 4, 5, 6 and 7 and in lanes 8, 9, 10, 11, 12 are 125 μM, 125 μM, 12.5 μM, 1.25 μM, and 0.125 μM, respectively. The reaction mixtures were added SDS loading dye (2% SDS), boil for 10 min, and run on SDS gel (Bio-Rad Any Kd gradient gel). The same gel was visualized by FAM fluorescence for DNA (top) and by Coomassie blue stain for protein (bottom).

FIG. 8A depicts the synthesis of BFPX probe, SPB-PEG3-AAD.

FIG. 8B is an ESI mass spectrum confirming the synthesized SPB-PEG3-AAD.

FIG. 8C depicts denaturing SDS gel analysis of covalent capture of in vitro assembled MEF2/DNA and NFAT1/DNA complexes by the BFPX probe, SPB-PEG3-AAD. Reactions of MEF2/DNA (lanes 1-4) and NFAT1/DNA (lanes 5-8) areas described in FIG. 7. The concentrations of SPB-PEG3-AAD in lanes 1, 2, 3, and 4 and in lanes 5, 6, 7, and 8 are 100 μM, 100 μM, 10 μM, 1 μM, respectively. The reaction mixtures were run on SDS gel and visualized by FAM fluorescence for DNA (top) and by Coomassie blue stain for protein (bottom) as described in FIG. 7.

FIG. 9A-FIG. 9D depicts characterization of BFPX-mediated crosslinking reaction, to identify the covalent attachment site on the protein. (FIG. 9A) A large scale of crosslinking reaction corresponding to lane 11 of FIG. 7 was carried out. The reaction mixture was separated on FPLC using a mono-Q column (yellow trace: conductance, red trace: salt gradient (A:10 mM Hepes pH 7.4; B: 10 mM Hepes 7.4, IM NaCl), blue trace: UV254 nm absorbance). (FIG. 9B) SDS PAGE analyses of the reaction and purification: lane 1, 5% reaction without UV 365 nm illumination, lane 2 and 3, two different batches of the crosslinking reaction, in addition to the free NFAT and DNA, a larger complex (presumably the covalent NFAT/DNA complex) appeared in lane 2 and 3 after UV365 nm crosslinking, which can be observed by fluorescent imaging (FAM, top) and the CCB-G250 stain (protein-stain, bottom) (interestingly free NFAT is also observable by fluorescent imaging). The flow-through contains only free NFAT (lane 4), peak I contains predominantly the complex (lane 5) and peak II contains predominantly free DNA (lane 6). (FIG. 9C) The purified NFAT/DNA complex was digested with trypsin and the DNA-peptide conjugate was purified and sequenced by Edman degradation: Top panel, a schematic picture of the DNA binding domain of human NFAT1 (residue 399-676) used in the study, the sequence of loop 478-491, RITGKTVTTTSYEK (SEQ ID NO.: 1), is shown above; Middle panel: a schematic picture of the covalently capture NFAT/DNA complex, the DNA sequence (5′-CCATAGAGGAAAATTTGTTTCATACAG-3′ (SEQ ID NO: 2) and its complementary strand 3′-GGTATCTCCTTTTAAACAAAGTATGTC-5′ (SEQ ID NO: 3)) used in the study is shown with the two potential probe (yellow wedge and start) binding site (TpA) indicated). The underlined region is the NFAT binding motif. Bottom panel: Edman sequencing results (1479, T480, and G481) identify the loop of 478-491, RITGKTVTTTSYEK (SEQ ID NO: 1), as the attachment site. (FIG. 9D) The biochemical results of (a-c) matches perfectly with the structural model wherein NFAT binds to it consensus GGAAAA motif underlined in SEQ ID NO: 2 and the BFPX probe insert into the adjacent TpA site.

FIG. 10A depicts Western blot analysis of presence of complexing of MEF2A with DNA in GM12878 cells treated with UV365 and SPB-PEG3-AAD. For each lane, one million of GM12878 cells were used. The nuclei of the cells were extracted and resuspended in 500 μl PBS, treated with various combination of SPB-PEG3-AAD (10 μM) and/or UV365 (10 min, 30 W, LED, sample distance 3 cm, cooling at 4-degree C.). The samples were then sonicated using covaries (1 min) followed by limited MNase digestion (10 min, 37-degree C.). The lysed and partially digested samples were then mixed with SDS loading dye (2% SDS, boil for 10 min) and run on SDS gel for western blot analyses of MEF2A using Anti-MEF2 antibody (B-4) (SCBT, cat #sc-17785).

FIG. 10B depicts Western blot analysis of Hela cells transfected with AVI-TEV-FLAG tagged FOXP3. For each lane, approximately one million of cells were used. Lane 1 was fixed with 1% formaldehyde (FA) for 10 min according to standard protocol. Lane 2, untreated control, Lane 3, UV only, lane 4 and lane 6 were treated with SPB-AAD of 1 or 10 μM, respectively but without UV illumination, lane 5 and lane 7 were treated with SPB-AAD of 1 or 10 μM, respectively and illuminated by UV365 (10 min, 30 W, LED, sample distance 3 cm, cooling at 4 degree C.). The cells were lysed using RIPA buffer and further digested by MNase. The sample mixtures were mixed with SDS loading dye (2% SDS, boil for 10 min) and run on SDS gel for western blot analyses of using Anti-FLAG antibody.

FIG. 11A-FIG. 11F depicts permeability and subcellular distribution of the BFPX probe SPB-AAD in Hela cell. Top panel (FIG. 11A-FIG. 11C): buffer control experiments without adding SPB-AAD, (FIG. 11A) bright field; (FIG. 11B) fluorescence imaging of Alexa Fluor; (FIG. 11C) DAPI stain of the nuclei; Bottom panel: treatment with 25 SPB-AAD, (FIG. 11D) bright field; (FIG. 11E) fluorescence imaging of Alexa Fluor; (FIG. 11F) DAPI stain of the nuclei.

FIG. 12A depicts the general design of the bi-functional photo-crosslinking (BFPX) strategy.

FIG. 12B depicts Psoralen-based BFPX probes synthesized and characterized in this study. The detailed information of compound synthesis and related spectroscopic validation are provided in the Examples section herein.

FIG. 12C depicts Native EMSA assay of the effect of Formaldehyde (FA), BFPX probe (SPB-AAD, SPB-PEG4-AAD, and SPB-Spermidine-AD) at various concentrations, and UVA (365 nm) on the DNA binding by NFAT1. The binding reactions were performed in a buffer of 20 mM Hepes pH 7.6, 150 mM NaCl, 1 mM DTT, 12% glycerol. Typically protein and DNA are mixed to bind at room temperature for 30 min, followed by addition of BFPX probes for an additional 15 min and then UV illumination. In the case of FA treatment, the reaction time is 10 min at room temperature and then quenched by 250 mM glycine. When included (as indicated by a positive sign), the binding reaction contains 10 μM 27 mer dsDNA (Cy5-labeled) with a NFAT-binding site (the antigen receptor response element 2, ARRE2, from the IL-2 promoter, 5′-Cy5-CCATAGAGGAAAATTTGTTTCATACAG-3′ (SEQ ID NO: 2), the complementary strand is not labeled), 24 μM of the DNA binding domain (DBD) of human NFAT1 protein (residue 399-676, NFAT1-DBD). The UV illumination (as indicated by a positive sign) was carried out using a LED Chip (21.4 mm×21.4 mm, 365-370 nm, 30 Watt, sample distance 2 cm, exposure time 2-15 min at 4 degree C., here the exposure time is 15 min). Half of the binding reaction (7.5 μl) was run on a 10% PAGE 0.5×TBE gel and visualized by Cy5 fluorescence on DNA. The small amount of single stranded DNA is due to incomplete annealing. The higher smearing bands are most likely non-specific protein complexes. Neither of these experimental imperfections affected the main conclusion of this experiment because the double stranded-DNA bands and the specific NFAT-DNA complex bands (the middle sharp bands) are well defined and can be monitored without the interference of the other bands.

FIG. 12D depicts the other half of binding reaction from the above experiments (FIG. 12C) were added SDS loading dye (2% SDS), boil for 10 min, and run on SDS gel (4-15% gradient gel). The gel was visualized by Cy5 fluorescence for DNA.

FIG. 12E depicts further characterization of BFPX crosslinking of NFAT1/DNA complexes using different DNA substrates and staining techniques. Two unlabeled DNA substrates containing a core NFAT-binding site and different flanking bases and sequence lengths (c1: 5′-TTCCACAGAGGAAAATTTGTT-3′ (SEQ ID NO: 4), 21 mer, c2: 5′-GTAGAGGAATTTCCTA-3′ (SEQ ID NO: 5), 16 mer) were used to bind the NFAT1-DBD as described in FIG. 12C. The binding reactions were then subject to BFPX crosslinking using SPB-AAD (100 μM) with (lanes 2, 4) and without (lanes 1 and 3) UV illumination. The reactions were then analyzed by SDS PAGE under denaturing condition as described in FIG. 12D. Because the DNA is unlabeled, the gel was stained using either cyanine-based dye (SYBR Safe) or Coomassie Blue stain (CB). As shown in the upper panel, NFAT was crosslinked to c1 DNA (lane 2) and c2 DNA (lane 4) in the presence of UV but not in the absence of UV illumination (lane 1 and lane 3, respectively). While the SYBR safe stained the NFAT-c1DNA complex and NFAT-c2DNA complex well (SYBR stained), both complexes were stained weakly in the Coomassie Blue stained gel (CB stained) wherein the free NFAT1 proteins were stained very well. The different size of the NFAT-c1DNA complexes (lane 2) and NFAT-c2DNA complexes (lane 4) was resolved by loading reactions of lane 2 (2a, 2b and 2c) and lane 4 (4a, 4b and 4c) as triplicates side-by-side on the same gel (lower panel) using either SYBR and CB stain.

FIG. 12F depicts time course of BFPX crosslinking of NFAT1/DNA complexes. The binding reactions between NFAT1 DBD and a 27 mer 5′6-FAM labeled ARRE2 DNA were set up as described in FIG. 12C either with (+) or without (−) the BFPX probe SPB-AAD (100 μM) and illuminated with UV for different amount of time: 3 seconds (3 s), 10 seconds (10 s), 30 seconds (30 s), 100 seconds (100 s), 5 min (5 m), 15 min (15 m), 30 min (30 m). The reactions at different time points were then analyzed on SDS gel as described in FIG. 12D. The crosslinked NFAT-DNA complex was visible as early as 3 seconds and reached plateau around 100 seconds and 5 min time points. When the UV exposure time is longer than 5 min, a significant amount of crosslinked NFAT-DNA complex can be formed with UV illumination without SPB-AAD.

FIG. 12G depicts time course of BFPX crosslinking of MEF2/DNA complexes. We also carried out the time course study of BFPX-based crosslinking of another transcription factor, the myocyte enhancer factor 2 (MEF2). The binding reactions between human MEF2A DBD (residues 2-95) and a 44 mer 5′6-FAM labeled DNA containing a consensus MEF2 site (5′-CTATAAATAG-3′, (SEQ ID NO: 6)) were set up as described in FIG. 12C either with (+) or without (−) the BFPX probe SPB-AAD (100 μM) and illuminated with UV for different amount of time: 3 seconds (3 s), 10 seconds (10 s), 30 seconds (30 s), 100 seconds (100 s), 5 min (5 m), 15 min (15 m), 30 min (30 m). The reactions at different time points were then analyzed on SDS gel as described in FIG. 12D. Again, the crosslinked MEF2-DNA complex was visible as early as 3 seconds and reached plateau around 100 seconds and 5 min time points. Unlike the NFAT/DNA complex, even with long UV exposure time of up to 30 min), no crosslinked MEF2/DNA complex was observed in the absence of the BFPX probe (SPB-AAD). This observation suggests that crosslinking between MEF2 and DNA is strictly dependent on the use of BFPX probe and that crosslinking of in is longer than 5 min, a significant amount of crosslinked NFAT-DNA complex can be formed with UV illumination without SPB-AAD.

FIG. 12H depicts BFPX crosslinking of Nkx2.5/DNA complexes. The binding reactions between human Nkx2.5 DBD (the homeodomain) and a 19 mer 5′6-FAM labeled DNA containing a consensus Nkx2.5 binding site (5′6-FAM-ACTATTTTAAGAACGTGCT-3′, (SEQ ID NO: 7)) were set up and treated with Formaldehyde (FA), or BFPX probes and UV illumination as described in FIG. 12C. The various treatment combinations were list above the gel. The reactions were then analyzed by SDS PAGE under denaturing condition as described in FIG. 12D. All three probes, SPB-AAD (S, lane 5), SPB-PEG4-AAD (E, lane 7), and SPB-Spermidine-AD (M, lane 9) showed significant crosslinking of the Nkx2.5/DNA complex while displaying little effect on the DNA alone (lane 4, 6 and 8). Once again, FA did not result in any protein-DNA crosslinking (lane 2) as compared with the control (lane 1) which contains only free DNA. UV alone in the absence of BFPX probe (lane 3) did not result in any Nkx2.5/DNA crosslinking (lane 3).

FIG. 12I depicts BFPX crosslinking of p53/DNA complexes. The binding reactions between the human tumor suppressor p53 DBD and a 37 mer Cy5 labeled DNA containing a consensus p53 tetramer binding site (5′-/5Cy5/-GCTAGCGTAGCAGGCATGCCTAGGCATGCCTGCTACG-3′, (SEQ ID NO: 8)) were set up and treated with BFPX probes and UV illumination as described in FIG. 12C. The various treatment combinations were list above the gel. The reactions were then analyzed by SDS PAGE under denaturing condition as described in FIG. 12D. All three probes, SPB-AAD (S, lane 2), SPB-PEG4-AAD (E, lane 4), and SPB-Spermidine-AD (M, lane 6) showed significant crosslinking of the p53/DNA complex as compared with the no-protein controls (lanes 1, 3 and 5). Because p53 bind this this DNA substrate as a tetramer, crosslinked p53/DNA complexes may contain one or more p53 protein molecules. While the monomeric p53/DNA complex appears to be the dominant crosslinked protein-DNA complexes, higher-molecule weight species were visible in SDS gel, which may correspond to crosslinked higher-order p53/DNA complex.

FIG. 12J depicts reversing BFPX crosslinking using a probe containing a cleavable linker, 4AMT-SDAD. Because UV alone can cross link NFAT1 to DNA at a low by significant level (FIG. 12F) but not MEF2 (FIG. 12G). We chose the MEF2/DNA complex for this test. The binding reactions between human MEF2A DBD (residues 2-95, 26 μM for all reactions) and a 16 mer 5′6-FAM labeled DNA containing a consensus MEF2 site (5′-CTATAAATAG-3′ (SEQ ID NO: 6), 0 μM for all reactions) were set up as described in FIG. 12C with increasing amount of 4AMT-SDAD from lane 3-1 and lane 6-4 at 10 μM, 50 μM and 100 μM, respectively. The binding reactions were then illuminated with UV for 15 min. The binding reactions of lane 4, 5, and 6 were further incubated with 200 mM TCEP at 37° C. overnight. All reactions were then analyzed on SDS gel as described in FIG. 12D. The result showed that the crosslinked MEF2/DNA complexes can be released by reductive cleavage of the disulfide bond in the linker of 4AMT-SDAD used in the crosslinking reaction.

FIG. 13A depicts characterization of the BFPX-mediated crosslinking reaction: Identification of covalent attachment sites on the crosslinked proteins. The NFAT1/DNA complex crosslinking reactions with SPB-AAD (corresponding to lane 8 of FIG. 12C) and SPB-PEG4-AAD (corresponding to lane 11 of FIG. 12C) were scaled up by 10 fold. A schematic picture of the DNA binding domain of human NFAT1 (residue 399-676) used in the study is shown at the top; the sequences of loop 434-452 (KAPTGGH (SEQ ID NO:9) . . . MENK (SEQ ID NO:10)) and loop 478-497 (RITGKTV (SEQ ID NO: 11) . . . IVGNTK(SEQ ID NO:12))are indicated. The reaction mixtures were separated on FPLC using a mono-Q column (yellow trace: conductance, red trace: salt gradient (buffer A:10 mM Hepes pH 7.4; buffer B: 10 mM Hepes 7.4, 1 M NaCl), blue trace: UV254 nm absorbance). The purified NFAT/DNA complex (P-D complex) was digested with trypsin and the DNA-peptide conjugate was purified and sequenced by Edman degradation. The DNA sequence (5′-CCATAGAGGAAAATTTGTTTCATACAG-3′ (SEQ ID NO: 2) and its complementary strand 3′-GGTATCTCCTTTTAAACAAAGTATGTC-5′ (SEQ ID NO: 3)) used in the study is shown with the two potential probe (yellow wedge and star) binding site (TpA) indicated. The underlined region is the NFAT binding motif. Edman sequencing of the linked peptide identified IVGN (SEQ ID NO: 13) and APTGGH (SEQ ID NO: 14).

FIG. 13B depicts the Edman sequencing results (IVGN) from the SPB-AAD crosslinked NFAT1/DNA complex identify the loop of 478-497 (colored in cyan) adjacent to DNA as the attachment site. This result is concordant with the structural model wherein NFAT binds to its consensus GGAAAA motif and the SPB-AAD probe (in space filling model) inserts into the adjacent TpA site.

FIG. 13C depicts the Edman sequencing results (APTGGH)(SEQ ID NO: 14) from the SPB-PEG4-AAD crosslinked NFAT1/DNA complex identify the loop of 434-452 (colored in magenta) as the attachment site. Loop of 434-452 is further away from DNA as compared with the loop of 478-497. This result is concordant with the structural model wherein NFAT binds to its consensus GGAAAA motif and the SPB-PEG4-AAD probe (in space filling model) inserts into the adjacent TpA site and adopt a largely extended conformation to each out to the 434-452 loop. Thus, the biochemical results matches very well with the structural model, demonstrating the potential utility of BFPX crosslinking in mapping the protein-DNA interface structure of in vitro assembled and in situ isolated transcription factor DNA complexes.

FIG. 14A depicts characterization and testing of BFPX crosslinking inside cells. Hela cells were incubated with SPB-AAD or SPB-PEG4-AAD (10 μM or 100 μM) in the dark for 30 min to allow the DNA binding and intercalation by the psoralen moiety of the BFPX probes. After washing off the excessive BFPX probe, illuminate UV 365 nm for 5 min. An Alexa Fluor 647 picolyl azide molecule (from Click-iT™ Plus Alexa Fluor™ 647 Picolyl Azide Toolkit) was used to react with the alkyne group on SPB-AAD or SPB-PEG4-AAD via click reaction. After the click chemistry labeling, the unreacted fluorescent molecules were removed by washing. The nuclei were analyzed using fluorescence imaging. When no BFPX probes were added (columns 1 and 2) or adding probes without UV treatment (columns 3 and 6), little or no fluorescence signal was retained in the cells. By contrast, only when the BFPX probes were present and activated by UV crosslinking, the Alexa Fluor 647 picolyl azide molecule could be immobilized in the cells via click reaction with SPB-AAD (columns 4 and 5) or SPB-PEG4-AAD (columns 7 and 8).

FIG. 14B depicts quantitative analyses showing that the immobilized fluorescent signal is dosage dependent on the concentration of SPB-AAD and SPB-PEG4-AAD under UV illumination.

FIG. 14C depicts 10 cm plate HEK293T cells (˜8.8 million) were transfected with 2 μg AVI-TEV-FLAG-FOXP3 for about 48 hr. The cells were harvested into 1.6 ml room temperature 1×DPBS buffer (with no calcium and magnesium). The cells were aliquoted into 300 μl per sample and each sample was added SPB-AAD 10 μM (A10) or 100 μM (A100) or SPB-PEG4-AAD 100 μM (p100). The samples were incubated at 37° C. for 30 min in dark with rotation. The samples were transferred to the middle wells of a 24-well plate (Corning #3527). With 300 μl, the liquid is about 1.5 mm high in a 24-well plate well. The samples on the 24-well plate were then UV illuminated (LED Chip: 21.4 mm×21.4 mm, 365-370 nm, 30 Watt, sample distance (the bottom of the well plate to the lamp plane) is 4 cm, exposure time 2-5 min at 4 degree C.). The BFPX-treated cells were recovered from the 24-well plate and washed using 1×DPBS, centrifuged at 500 g for 5 min to pellet the cells and remove the DPBS buffer. 0.5 million of the BFPX-treated cells was added 20 μl 1×RIPA buffer, shake on ice for 15 min to lyse the cells. The lysis mixture was then added 3 μl 10× MNase, 0.25 μl RnaseA, 12.5 Unit Mnase, and ddw to 30 μl, incubated at 37° C. for 15 min. The lysis mixture was then added 6 μl SDS loading dye (2% SDS), boil 10 min at 95° C. Run at 200V for 40 minutes on 4%-15% SDS-PAGE gel and analyzed by western blot (Transfer at 18V (30 mA) overnight, block in 2% BSA in PBS for 45 min, Primary antibody: 1:2000 anti-FLAG; room temperature, 2 hr; Secondary antibody: anti mouse light chain kappa 1: 100,000; Femto Signal ECL). As shown in FIG. 14C, higher molecular weight species containing FOXP3 were observed in SPB-AAD (A) and SPB-PEG4-AAD (P) treated cells (lanes 4-9) as compared with controls (lanes 1-3), and the formation of crosslinked FOXP3 complexes is dosage dependent on probe concentration (compare lane 4 (SPB-AAD, 10 μM, A10), and lane 6 (SPB-AAD, 100 μM, A100)) and the UV exposure time (compare lane 4 (2 min) and lane 5 (5 min) at SPB-AAD concentration of 10 μM (A10); also compare lane 8 (2 min) and lane 9 (5 min) at SPB-PEG4 AAD concentration of 100 μM (P100).

FIG. 14D depicts for the same BFPX-treated HEK293T cells transfected with AVI-TEV-FLAG-FOXP3 described in (FIG. 14C), TRIzol was used to extract genomic DNA. Briefly, for each aliquot of 0.25 million of cells, lyse and homogenize the sample in 200 μl TRIzol™ Reagent. Incubate for 5 minutes to permit complete dissociation of the nucleoproteins complex. 40 μl of chloroform was then added to the sample to incubate for 3 min. The sample was then centrifuged for 15 minutes at 12,000×g at 4° C. to separate into a lower red phenol/chloroform-interphase layer and a colorless upper aqueous phase. The upper aqueous phase was removed by pipetting. 60 μl 100% ethanol was added to the remaining organic phase and interface layer, mixing well, incubating for 3 min, followed by centrifugation for 5 min at 2,000×g at 4° C. to pellet the genomic DNA. Resuspend the DNA pellet in 200 μl of 0.1 M sodium citrate/10% ethanol (pH 8.5), incubate for 30 min., then centrifuge for 5 min at 2,000×g at 4° C. to pellet the genomic DNA again. Repeat the 0.1 M sodium citrate/100% ethanol wash one more time. Resuspend the DNA in 400 μl 75% ethanol, incubate 20 min., centrifuge for 5 min at 2,000×g at 4° C. to pellet the genomic DNA. Air dry the DNA pellet for 10 min. Dissolve the DNA pellet in 300 μl freshly made 8 mM NaOH. Once the DNA is dissolved, adjust the pH to ˜pH 7.5 using 3 M sodium acetate (pH 5.2, about 10-11 μl added). The genomic DNA sample was then added 10× Benzonase buffer (final conc 1×) and 0.2 Unit of Benzonase to digest the genomic DNA completely (checked by agarose gel). The sample was then added SDS loading dye (2% SDS), boil 10 min at 95° C. Run at 200V for 40 minutes on 4%-15% SDS-PAGE gel and analyzed by western blot (Transfer at 20V (40 mA) overnight, block in 2% BSA in PBS for 45 min, Primary antibody: 1:2000 anti-FLAG; room temperature, 2 hr; Secondary antibody: anti mouse light chain kappa 1:100,000; SuperSignal™ Western Blot Substrate Bundle, Femto). As shown in FIG. 14D, only cells treated with UV and probe (SPB-AAD, 100 μM, A100) showed FOXP3 proteins (lanes 4 and 5) as compared with the controls (lane 1-3). This observation suggests that FOXP3 proteins were covalently linked to genomic DNA in BFPX treated cells that can be extracted by the TRIzol protocol under strong denaturing conditions whereas in control cells the non-covalent nucleoprotein complexes were completely dissociated. Moreover, Benzonase digestion releases not only FOXP3 with a molecular weight similar to its free monomer but also higher molecular species, suggesting that FOXP3 may bind some genomic region as higher order structures that are resistant to Benzonase digestion.

FIG. 14E depicts capture of the endogenous transcription factor MEF2 bound to genomic DNA in GM12878 cells. GM12878 cells (7.5 million for each sample) were treated with different combinations of UV illumination and BFPX probe, and the genomic DNA was extracted using the TRIzol protocol, digested by Benzonase and analyzed by SDS-PAGE/western blot as described in FIG. 14D. Here the Trizol extraction procedure was scaled up to 7.5 million cells per ml of TRIzol reagent. The final DNA pellet was dissolved in 300 μl 8 mM NaOH and neutralized with 10-11 μl 3 M sodium acetate (pH 5.2) and digested using 25 U of Benzonase for 1 hour. The results showed that MEF2C covalently bound to genomic DNA and extracted by TRIzol under strong denaturing condition were only observed on cells treated with UV and probe (lanes, 3, 4 and 5) as compared with the control (lane 1). The UV only control (lane 2) showed a weak signal, suggesting that UV alone may crosslink some protein to DNA at a low level. But the crosslinking efficiency in BFPX-treated cells is significantly higher than the UV only control (compare lanes 3-5 and lane 2).

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed, Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application. For references on how to prepare antibodies, see D. Lane, Antibodies: A Laboratory Manual 2^{nd} ed (Cold Spring Harbor Press, Cold Spring Harbor NY, 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For purposes of the present invention, the following terms are defined below. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, systems, articles of manufacture, apparatus, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

In some embodiments, the numbers expressing quantities of reagents, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

As used herein the term “electron donating group” is well-known in the art and generally refers to a functional group or atom that pushes electron density away from itself, towards other portions of the molecule, e.g., through resonance and/or inductive effects. Non-limiting examples of electron-donating groups include OR^{c}, NR^{c}R^{d}, alkyl groups, wherein R^{c} and R^{d} are each independently H, optionally substituted alkyl, optionally substituted heteroalkyl, optionally substituted aryl, optionally substituted heteroaryl, optionally substituted cyclyl, or optionally substituted heterocyclyl.

As used herein the term “electron withdrawing group” is well-known in the art and generally refers to a functional group or atom that pulls electron density towards itself, away from other portions of the molecule, e.g., through resonance and/or inductive effects. Non-limiting examples of electron withdrawing groups include NO₂, F, Cl, Br, I, CF₃. CN, CO₂R^{a}, C(═O)NR^{a}R^{b}, C(═O)R^{a}, SO₂R^{a}, SO₂OR^{a}, SO₂NR^{a}R^{b}, PO₃R^{a}R^{b}, or NO, wherein R^{a} and R^{b} are each independently H, optionally substituted alkyl, optionally substituted heteroalkyl, optionally substituted aryl, optionally substituted heteroaryl, optionally substituted cyclyl, or optionally substituted heterocyclyl.

As used herein, the term “alkyl” means a straight or branched, saturated aliphatic radical having a chain of carbon atoms. Cₓ alkyl and Cₓ-C_{y} alkyl are typically used where X and Y indicate the number of carbon atoms in the chain. For example, C₁-C₆ alkyl includes alkyls that have a chain of between 1 and 6 carbons (e.g., methyl, ethyl, propyl, isopropyl, butyl, sec-butyl, isobutyl, tert-butyl, pentyl, neopentyl, hexyl, and the like). Alkyl represented along with another radical (e.g., as in arylalkyl) means a straight or branched, saturated alkyl divalent radical having the number of atoms indicated or when no atoms are indicated means a bond, e.g., (C₆-C₁₀)aryl(C₀-C₃)alkyl includes phenyl, benzyl, phenethyl, 1-phenylethyl 3-phenylpropyl, and the like. Backbone of the alkyl can be optionally inserted with one or more heteroatoms, such as N, O, or S.

In preferred embodiments, a straight chain or branched chain alkyl has 30 or fewer carbon atoms in its backbone (e.g., C1-C30 for straight chains, C3-C30 for branched chains), and more preferably 20 or fewer. Likewise, preferred cycloalkyls have from 3-10 carbon atoms in their ring structure, and more preferably have 5, 6 or 7 carbons in the ring structure. The term “alkyl” (or“loweralkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

Non-limiting examples of substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like.

As used herein, the term “alkenyl” refers to unsaturated straight-chain, branched-chain or cyclic hydrocarbon radicals having at least one carbon-carbon double bond. Cₓ alkenyl and Cₓ-C_{y} alkenyl are typically used where X and Y indicate the number of carbon atoms in the chain. For example, C₂-C₆alkenyl includes alkenyls that have a chain of between 2 and 6 carbons and at least one double bond, e.g., vinyl, allyl, propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S.

As used herein, the term “alkynyl” refers to unsaturated hydrocarbon radicals having at least one carbon-carbon triple bond. Cₓ alkynyl and Cₓ-C_{y} alkynyl are typically used where X and Y indicate the number of carbon atoms in the chain. For example, C₂-C₆alkynyl includes alkynyls that have a chain of between 2 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S.

The terms “alkylene,” “alkenylene,” and “alkynylene” refer to divalent alkyl, alkaline, and alkynylene” radicals. Prefixes C, and Cₓ-C_{y} are typically used where X and Y indicate the number of carbon atoms in the chain. For example, C₁-C₆alkylene includes methylene, (—CH₂—), ethylene (—CH₂CH₂—), trimethylene (—CH₂CH₂CH₂—), tetramethylene (—CH₂CH₂CH₂CH₂—), 2-methyltetramethylene (—CH₂CH(CH₃)CH₂CH₂—), pentamethylene (—CH₂CH₂CH₂CH₂CH₂—) and the like).

As used herein, the term “alkylidene” means a straight or branched unsaturated, aliphatic, divalent radical having a general formula=CRₐR_{b}. Non-limiting examples of Rₐ and R_{b} are each independently hydrogen, alkyl, substituted alkyl, alkenyl, or substituted alkenyl. C, alkylidene and Cₓ-C_{y} alkylidene are typically used where X and Y indicate the number of carbon atoms in the chain. For example, C₂-C₆ alkylidene includes methylidene (═CH₂), ethylidene (═CHCH₃), isopropylidene (═C(CH₃)₂), propylidene (═CHCH₂CH₃), allylidene (═CH—CH═CH₂), and the like).

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

As used herein, the term “halogen” or “halo” refers to an atom selected from fluorine (F), chlorine (Cl), bromine (Br) and iodine (I). The term “halogen radioisotope” or “halo radioisotope” refers to a radionuclide of an atom selected from fluorine (F), chlorine (Cl), bromine (Br) and iodine (I).

In some embodiments, “iodo” refers to the iodine atom (I) when it is used in the context of a halo functional group or halogen functional group or as a halo substituent or halogen substituent.

In some embodiments, “bromo” refers to the bromine atom (Br) when it is used in the context of a halo functional group or halogen functional group or as a halo substituent or halogen substituent.

In some embodiments, “chloro” refers to the chlorine atom (Cl) when it is used in the context of a halo functional group or halogen functional group or as a halo substituent or halogen substituent.

In some embodiments, “fluoro” refers to the fluorine atom (F) when it is used in the context of a halo functional group or halogen functional group or as a halo substituent or halogen substituent.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (—CF₃), 2,2,2-trifluoroethyl, perfluoroethyl, 2,2,2-trifluoro-1,1-dichloroethyl, and the like).

The term “aryl” refers to monocyclic, bicyclic, or tricyclic fused aromatic ring system. Cₓ aryl and Cₓ-C_{y} aryl are typically used where X and Y indicate the number of carbon atoms in the ring system. For example, C₆-C₁₂ aryl includes aryls that have 6 to 12 carbon atoms in the ring system. Exemplary aryl groups include, but are not limited to, pyridinyl, pyrimidinyl, furanyl, thienyl, imidazolyl, thiazolyl, pyrazolyl, pyridazinyl, pyrazinyl, triazinyl, tetrazolyl, indolyl, benzyl, phenyl, naphthyl, anthracenyl, azulenyl, fluorenyl, indanyl, indenyl, naphthyl, phenyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

The term “heteroaryl” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ heteroaryl and Cₓ-C_{y} heteroaryl are typically used where X and Y indicate the number of carbon atoms in the ring system. For example, C₄-C₉ heteroaryl includes heteroaryls that have 4 to 9 carbon atoms in the ring system. Heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazine, phenoxazine, 1,2-dihydropyrrolo[3,2,1-hi]indole, indolizine, pyrido[1,2-a]indole, 2(H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Some exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring may be substituted by a substituent.

The term “cyclyl” or “cycloalkyl” refers to saturated and partially unsaturated cyclic hydrocarbon groups having 3 to 12 carbons, for example, 3 to 8 carbons, and, for example, 3 to 6 carbons. Cₓ cyclyl and Cₓ-C_{y} cycyl are typically used where X and Y indicate the number of carbon atoms in the ring system. For example, C₃-C₈ cyclyl includes cyclyls that have 3 to 8 carbon atoms in the ring system. The cycloalkyl group additionally can be optionally substituted, e.g., with 1, 2, 3, or 4 substituents. C₃-C₁₀ cyclyl includes cyclopropyl, cyclobutyl, cyclopentyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo[2.2.1]hept-1-yl, and the like.

Aryl and heteroaryls can be optionally substituted with one or more substituents at one or more positions, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The term “heterocyclyl” refers to a nonaromatic 4-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓ heterocyclyl and Cₓ-C_{y} heterocyclyl are typically used where X and Y indicate the number of carbon atoms in the ring system. For example, C₄-C₉ heterocyclyl includes heterocyclyls that have 4-9 carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

The terms “bicyclic” and “tricyclic” refers to fused, bridged, or joined by a single bond polycyclic ring assemblies.

The term “cyclylalkylene” means a divalent aryl, heteroaryl, cyclyl, or heterocyclyl.

As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems can be saturated, partially saturated, cyclyl, heterocyclyl, aromatics, heteroaromatics, and the like.

As used herein, the term “carbonyl” means the radical —C(O)—. It is noted that the carbonyl radical can be further substituted with a variety of substituents to form different carbonyl groups including acids, acid halides, amides, esters, ketones, and the like.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. The term “carboxyl” means —COOH.

The term “cyano” means the radical —CN.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O′)═, —O—, —S— or —S(O)₂—, —OS(O)₂—, and —SS—, wherein RN is H or a further substituent.

The term “hydroxy” means the radical —OH.

The term “imine derivative” means a derivative comprising the moiety —C(NR)—, wherein R comprises a hydrogen or carbon atom alpha to the nitrogen.

The term “nitro” means the radical —NO₂.

An “oxaaliphatic,” “oxaalicyclic”, or “oxaaromatic” mean an aliphatic, alicyclic, or aromatic, as defined herein, except where one or more oxygen atoms (—O—) are positioned between carbon atoms of the aliphatic, alicyclic, or aromatic respectively.

An “oxoaliphatic,” “oxoalicyclic”, or “oxoaromatic” means an aliphatic, alicyclic, or aromatic, as defined herein, substituted with a carbonyl group. The carbonyl group can be an aldehyde, ketone, ester, amide, acid, or acid halide.

As used herein, the term, “aromatic” means a moiety wherein the constituent atoms make up an unsaturated ring system, all atoms in the ring system are sp² hybridized and the total number of pi electrons is equal to 4n+2. An aromatic ring can be such that the ring atoms are only carbon atoms (e.g., aryl) or can include carbon and non-carbon atoms (e.g., heteroaryl).

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbamoyl, alkylene, alkylidene, alkylthios, alkynyl, amide, amido, amino, aminoalkyl, aralkyl, aralkylsulfonamido, arenesulfonamido, arenesulfonyl, aromatic, aryl, arylamino, arylcarbamoyl, aryloxy, azido, carbamoyl, carbonyl, carbonyls including ketones, carboxy, carboxylates, CF₃, cyano (CN), cycloalkyl, cycloalkylene, ester, ether, haloalkyl, halogen, halogen, heteroaryl, heterocyclyl, hydroxy, hydroxyalkyl, imino, iminoketene, ketone, mercapto, nitro, oxoalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which may optionally also be substituted or unsubstituted. In some cases, two substituents, together with the carbon(s) to which they are attached to, can form a ring.

Substituents may be protected as necessary and any of the protecting groups commonly used in the art may be employed. Non-limiting examples of protecting groups may be found, for example, in Greene et al., Protective Groups in Organic Synthesis, 3rd Ed. (New York: Wiley, 1999).

The terms “alkoxyl” or “alkoxy” as used herein refers to an alkyl group, as defined above, having an oxygen radical attached thereto. Representative alkoxyl groups include methoxy, ethoxy, propyloxy, tert-butoxy, n-propyloxy, iso-propyloxy, n-butyloxy, iso-butyloxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aryoxy can be represented by —O-aryl or O-heteroaryl, wherein aryl and heteroaryl are as defined below. The alkoxy and aryoxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

As used herein, the term “amino” means —NH₂. The term “alkylamino” means a nitrogen moiety having at least one straight or branched unsaturated aliphatic, cyclyl, or heterocyclyl radicals attached to the nitrogen. For example, representative amino groups include —NH₂, —NHCH₃, —N(CH₃)₂, —NH(C₁-C₁₀alkyl), —N(C₁-C₁₀alkyl)₂, and the like. The term “alkylamino” includes “alkenylamino,” “alkynylamino,” “cyclylamino,” and “heterocyclylamino.” The term “arylamino” means a nitrogen moiety having at least one aryl radical attached to the nitrogen. For example —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The term “alkoxyalkoxy” means —O-(alkyl)-O-(alkyl), such as —OCH₂CH₂OCH₃, and the like.

The term “alkoxycarbonyl” means —C(O)O-(alkyl), such as —C(═O)OCH₃, —C(═O)OCH₂CH₃, and the like.

The term “alkoxyalkyl” means -(alkyl)-O-(alkyl), such as —CH₂OCH₃, —CH₂OCH₂CH₃, and the like.

The term “aryloxy” means —O-(aryl), such as —O-phenyl, —O-pyridinyl, and the like.

The term “arylalkyl” means -(alkyl)-(aryl), such as benzyl (i.e., —CH₂phenyl), —CH₂-pyrindinyl, and the like.

The term “arylalkyloxy” means —O-(alkyl)-(aryl), such as —O-benzyl, —O—CH₂-pyridinyl, and the like.

The term “cycloalkyloxy” means —O-(cycloalkyl), such as —O-cyclohexyl, and the like.

The term “cycloalkylalkyloxy” means —O-(alkyl)-(cycloalkyl, such as —OCH₂ cyclohexyl, and the like.

The term “aminoalkoxy” means —O-(alkyl)-NH₂, such as —OCH₂NH₂, —OCH₂CH₂NH₂, and the like.

The term “mono- or di-alkylamino” means —NH(alkyl) or —N(alkyl)(alkyl), respectively, such as —NHCH₃, —N(CH₃)₂, and the like.

The term “mono- or di-alkylaminoalkoxy” means —O-(alkyl)-NH(alkyl) or —O-(alkyl)-N(alkyl)(alkyl), respectively, such as —OCH₂NHCH₃, —OCH₂CH₂N(CH₃)₂, and the like.

The term “arylamino” means —NH(aryl), such as —NH-phenyl, —NH-pyridinyl, and the like.

The term “arylalkylamino” means —NH-(alkyl)-(aryl), such as —NH-benzyl, —NHCH₂-pyridinyl, and the like.

The term “alkylamino” means —NH(alkyl), such as —NHCH₃, —NHCH₂CH₃, and the like.

The term “cycloalkylamino” means —NH-(cycloalkyl), such as —NH-cyclohexyl, and the like.

The term “cycloalkylalkylamino”-NH-(alkyl)-(cycloalkyl), such as —NHCH₂— cyclohexyl, and the like.

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH₃) as well as —CRₐR_{b}R_{c} where Rₐ, R_{b}, and R_{c} can each independently be hydrogen or any other substituent where the atom alpha to the carbon is a heteroatom or cyano. Hence, CF₃, CH₂OH and CH₂CN are all C₁ alkyls.

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

In various embodiments, compounds of the present invention as disclosed herein may be synthesized using any synthetic method available to one of skill in the art. Non-limiting examples of synthetic methods used to prepare various embodiments of compounds of the present invention are disclosed in the Examples section herein.

“Hi-C” refers to a technique, in which chromatin is crosslinked with formaldehyde, then digested, and re-ligated in such a way that only DNA fragments that are covalently linked together form ligation products. It is based on Chromosome Conformation Capture, and can be used to comprehensively detect chromatin interactions in the mammalian nucleus.

“Diazirines” refer to a class of organic molecules consisting of a carbon bound to two nitrogen atoms, which are double-bonded to each other, forming a cyclopropene-like ring, 3H-diazirene. They are isomeric with diazocarbon groups, and like them can serve as precursors for carbenes by loss of a molecule of dinitrogen. For example, irradiation of diazirines with ultraviolet light leads to carbene insertion into various C—H, N—H, and O—H bonds. Without wishing to be bound by a particular theory, photo-activation of diazirine creates reactive carbene intermediates. Such intermediates can form covalent bonds through addition reactions with any amino acid side chain or peptide backbone (e.g., a protein or other molecule that contains nucleophilic or active hydrogen groups R′) at distances corresponding to the spacer arm lengths of the particular reagent.

In some embodiments, a diazirine is:

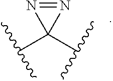 [SMILES: *C1(*)N=N1]

In some embodiments, a diazirine is:

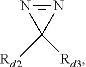

wherein R_{d2} and R_{d3} are each independently H, halo, CH₃, CF₃, optionally substituted alkyl, optionally substituted heteroalkyl, optionally substituted aryl, optionally substituted heteroaryl, optionally substituted cyclyl, or optionally substituted heterocyclyl.

In some embodiments, a diazirine is:

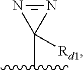

wherein R_{d1} is H, halo, CH₃, CF₃, optionally substituted alkyl, optionally substituted heteroalkyl, optionally substituted aryl, optionally substituted heteroaryl, optionally substituted cyclyl, or optionally substituted heterocyclyl.

Psoralen intercalates into the DNA double helix where it is ideally positioned to form one or more adducts with adjacent pyrimidine bases, preferentially thymine, upon excitation by an ultraviolet photon. Psoralens can also be activated by irradiation with long wavelength UV light. While UVA range light is the clinical standard, UVB is more efficient at forming photoadducts. The photochemically reactive sites in psoralens are the alkene-like carbon-carbon double bonds in the furan ring (the five-member ring) and the pyrone ring (the six-member ring). Without wishing to be bound by a particular theory, when appropriately intercalated adjacent to a pyrimidine base, a four-center photocycloaddition reaction can lead to the formation of either of two cyclobutyl-type monoadducts. Ordinarily, furan-side monoadducts form in a higher proportion. The furan monoadduct can absorb a second UVA photon leading to a second four-center photocycloaddition at the pyrone end of the molecule and hence the formation of a diadduct or cross-link. Pyrone monoadducts do not absorb in the UVA range and hence cannot form crosslinks with further UVA irradiation.

“Azo biotin-azide” refers to a linker that contains a biotin moiety connected to an azide group through a spacer arm containing a diazo group that can be cleaved with 50 mM sodium dithionite solution. Azo compounds are compounds bearing the functional group diazenyl R—N═N—R′, in which R and R′ can be either aryl or alkyl.

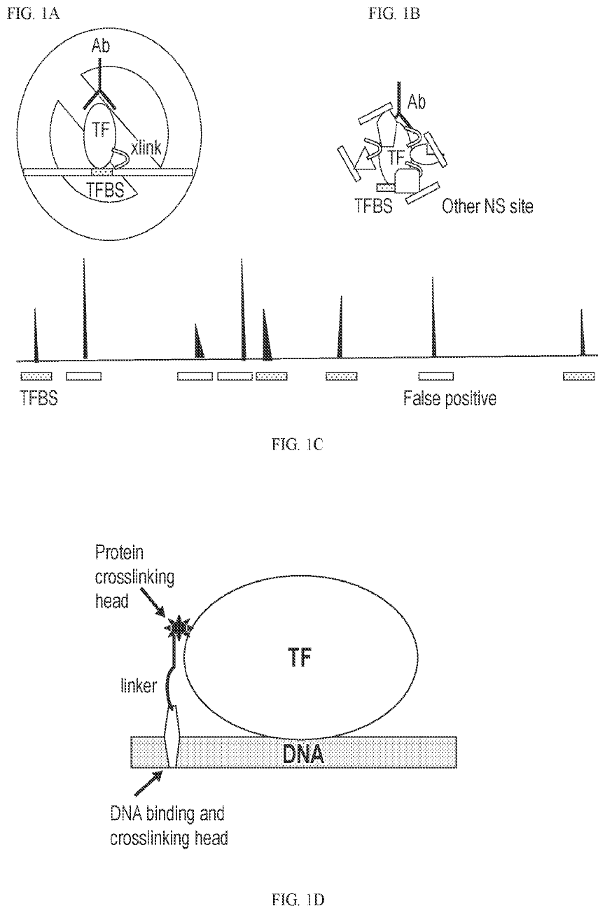

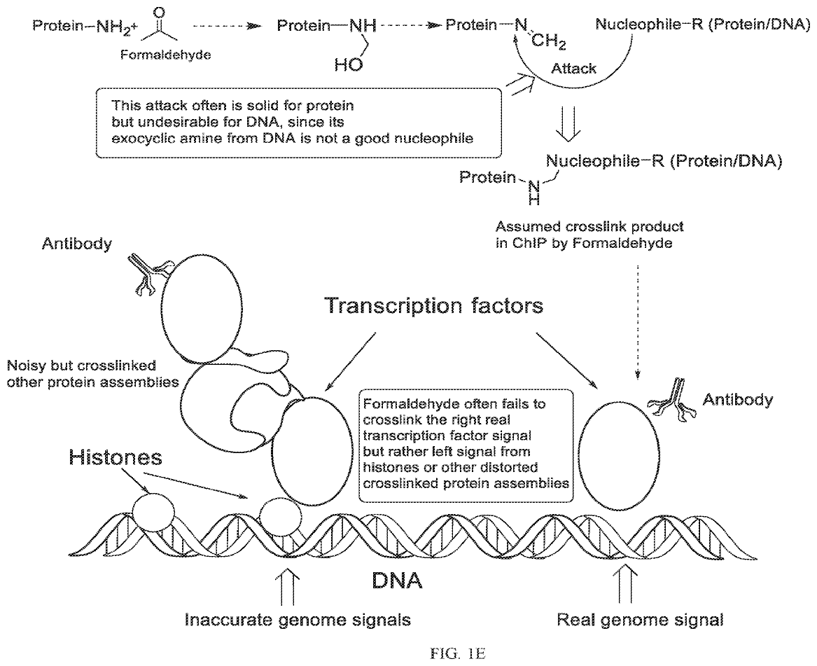

Herein, we have developed a class of molecular probes that bind DNA, and under illumination with long-wavelength UVA (˜360 nm), become activated to engage efficient crosslinking to DNA and proteins that are bound nearby (FIG. 1D). These molecular probes will serve as powerful tools for the covalent capture of protein-DNA and protein-RNA complexes in a wide range of in situ studies of protein-nucleic acid interactions. This new class of molecules, also called bifunctional photocrosslinking (BFPX) probes, permit selective, efficient, and robust (stable) capture of protein-nucleic acid complexes inside cells. Compared with the existing crosslinking technologies, the probes to be developed by the proposed research will have the following new and useful features: (1) High specificity: Unlike formaldehyde and other lysine-reacting bi-functional chemical crosslinking probes (e.g. DSS), which will react with any proteins in the cells, causing damage to antibody epitopes and DNA-binding surface of TFs, our probes will preferably bind only to nucleic acids by the nucleic acids specific recognition head, thereby achieving regioselectivity of photochemical reactions at or near the DNA binding site. (2) High efficiency: we can introduce photo-affinity labeling groups (such as diazirine) that are highly efficient and can be activated by long-wavelength UVA (˜360 nm), thereby reducing the photodamage associated with UVC (250 nm) irradiation. (3) Versatility and tunability: through synthetic engineering, variable functional heads or linkers can be introduced for customized applications. For example, multiple arms of photo-affinity labeling groups could be introduced to capture more than one TFs such as in the NFAT/Fos-Jun/DNA ternary complex, allowing for direct experimental determination of multi-TF complexes rather than relying on sequential ChIP-seq, which is not feasible for most TFs with the current approaches. The linker length can be varied to capture proximal DNA-binding domains or more distal protein cofactors recruited by the TF. (4) Temporal and spatial selectivity: The crosslinking can be initiated by controlled UV illumination in terms of timing and focus, thus allowing for potential temporal and spatial control to capture protein/DNA complexes in selected time and subcellular regions. (5) Allowing for the study of genome-bound-proteome: Because of the enhanced specificity of crosslinking only proteins bound to DNA, in addition to identifying DNA sequences bound by proteins of interests (such as traditional ChIP-seq), it is also possible to use a reversible linker, isotope labeling, and mass spectrometry to label and identify all proteins that are bound to DNA throughout the genome upon UV illumination (conceptually an inverse of ChIP-seq). This will provide unprecedented information for protein-DNA interactions at genome-wide and proteome-wide scales.

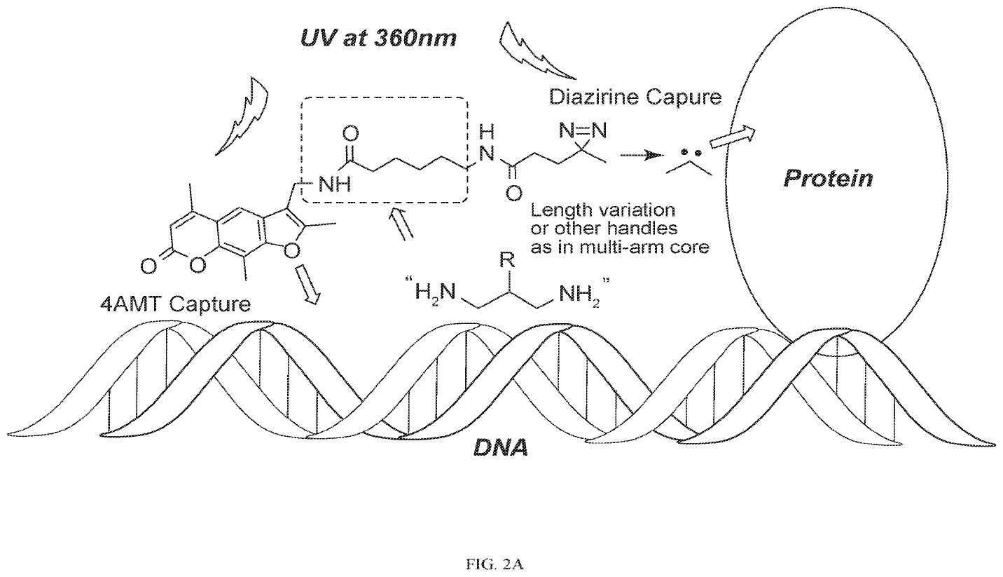

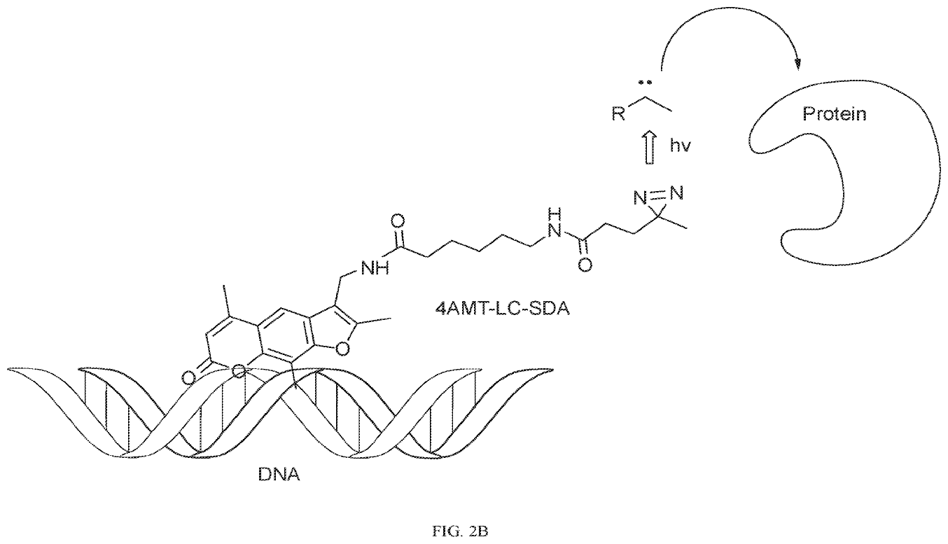

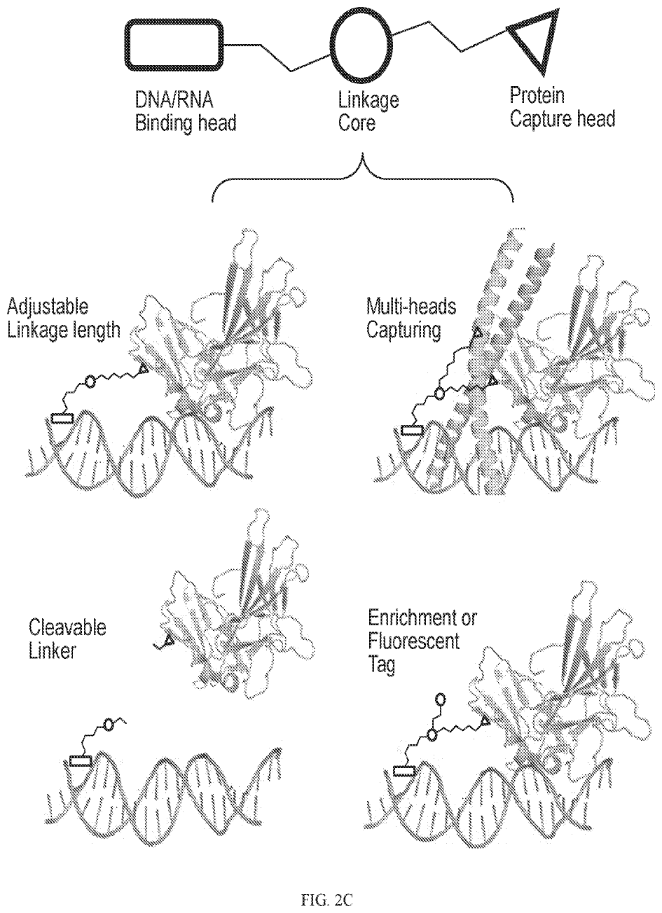

We have developed a class of molecular probes that bind DNA or RNA, and under illumination with long wavelength UV (˜350 nm or about 365 nm), become activated to engage efficient crosslinking to DNA and proteins that are bound nearby (FIG. 2A, FIG. 2B). These molecular probes will serve as powerful tools for covalent capture of protein-DNA and protein-RNA complexes with high efficiency, selectivity (i.e., only targeting DNA-bound proteins) and stability (to enable subsequent XL-MS analyses) in a wide range of in situ studies of protein-nucleic acid interactions.

To overcome the intrinsic disadvantages with formaldehyde crosslinking (it cannot directly crosslink protein to DNA, because the amine from DNA is not active), we have designed bifunctional probes to confer DNA molecules and/or RNA molecules) with real active primary amine. Through an amine linkage (e.g., amine NHS or click chemistry by converting amine to azide), a photo-reactive head (functional group) is introduced for crosslinking with nearby proteins, therefore truly and only capturing nucleic acids with proteins that are in close proximity. This gives more precise location of chromatin protein binding on DNA, as well as preserving epitopes on protein, as it does not act like formaldehyde to crosslink every protein they meet (in chromatin immunoprecipitation, ChIP, or in Hi-C). This can also give protein an ID for mass spectrometry identification (reverse ChIP).

Using DNA as an example, studying the binding of proteins to DNA throughout the genome inside the cells has been a key to understanding cellular mechanisms. A conventional step in these studies is to covalently fix the protein to DNA by formaldehyde crosslinking, such that the protein-bound DNA sites could be analyzed by ChIP-seq or that the protein-mediated chromatin interactions could be analyzed by Hi-C, HiChIP or similar technologies. However, increasing evidence indicates that formaldehyde crosslinking has major limitations, including non-specific modifications of proteins so as to destroy the epitopes of antibodies used in ChIPseq, or inefficient capture of transcription factors such as Lac repressors and NF-kappaB.

To overcome these limitations, we have developed a new approach to covalently capturing protein-nucleic acid complexes in situ using bi-functional photo-crosslinking probes. A class of new molecule tools are provided, which have two photo-crosslinking functional groups, hence the name bi-functional photo-crosslinking (BFPX).

In various embodiments, one such functional group, the DNA photo-crosslinking head, is composed of non-specific DNA binding small molecules (such as psoralen, DAPI, benzophenone etc—for genome wide capture of all protein/DNA complexes) or specific DNA binding small molecules (such as polyamide—for targeted capture of all protein/DNA complexes) that has either intrinsic ability to crosslink with DNA under long UV wavelength (˜360 nm, such as psoralen; or between 330 nm and 370 nm)) or contains a synthetically introduced photo-crosslinking moiety (e.g., benzophone or phenyl diazirine). Other embodiments provide that the nucleic acid-binding head/functional group is derived from methyltrioxsalen; a Hoechst dye, e.g., Hoechst 33342 (2′-[4-ethoxyphenyl]-5-[4-methyl-1-piperazinyl]-2,5′-bi-1H-benzimidazole trihydrochloride trihydrate), Hoechst 33258, Hoechst 34580, Hoechst S769121; a polyamide; or a G quartet binding molecule.

The DNA crosslinking head will be connected through a linker to a protein capture photo-crosslinking head that can be either a single arm of diazirine or multiple arms of several diazirine groups (for multiple captures of more than one proteins bound to DNA together).

The linker length can be of variable length for capture of proximal direct DNA-binding proteins (shorter linker length) or protein co-factors further away from DNA but recruited by DNA binding proteins (longer linker length), and to improve capturing efficiency. (Too short of a linker will lead to crosslinking back to DNA, whereas too long of a linker will capture non-specific proteins not associated with DNA directly or indirectly). The linker can also be engineered to contain tags (such as an alkyne group) that can be used for enrichment of the captured protein/DNA complexes through click chemistry, or for fluorescent labeling to track the probe distributions inside the cells by azido-fluorophore.

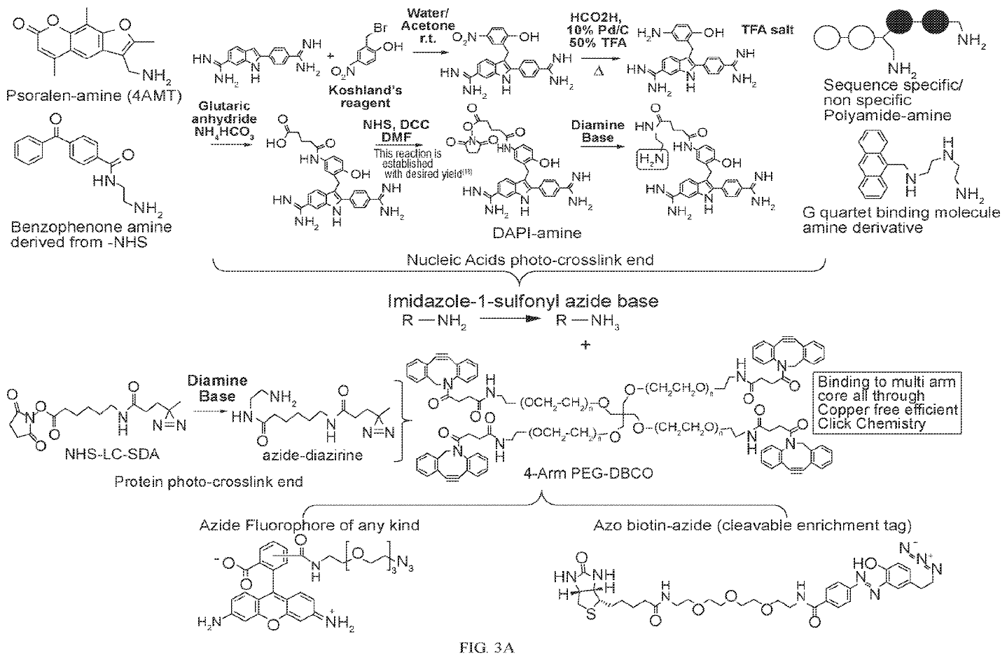

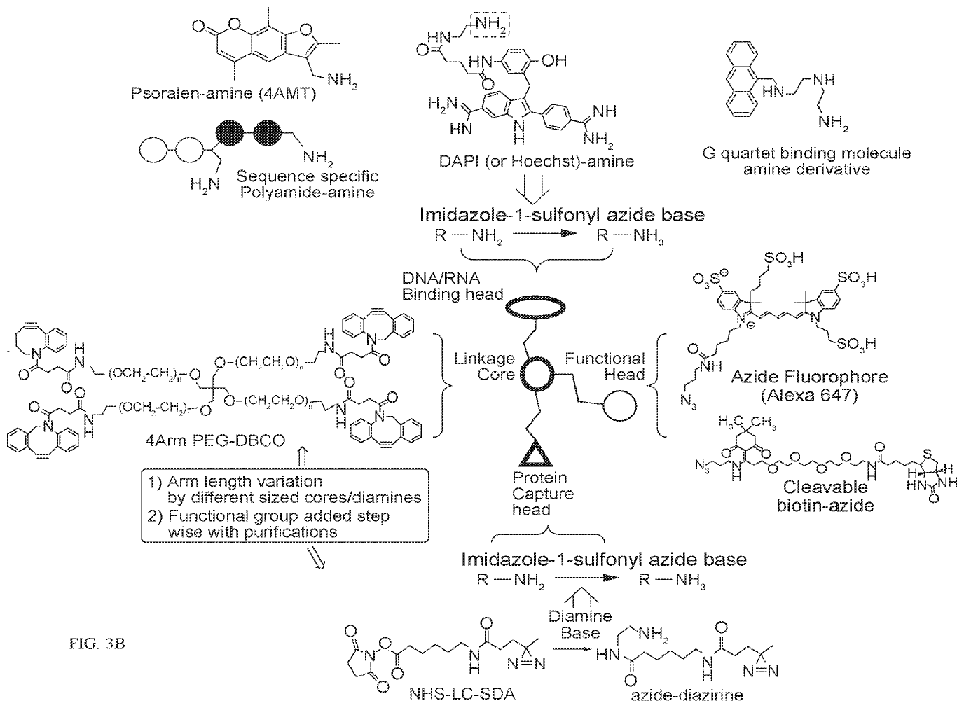

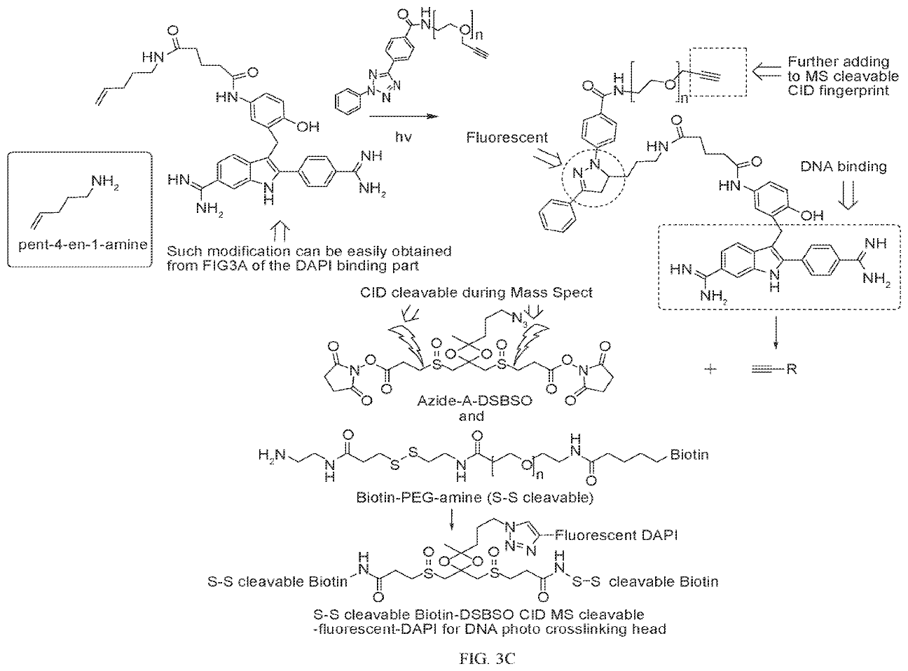

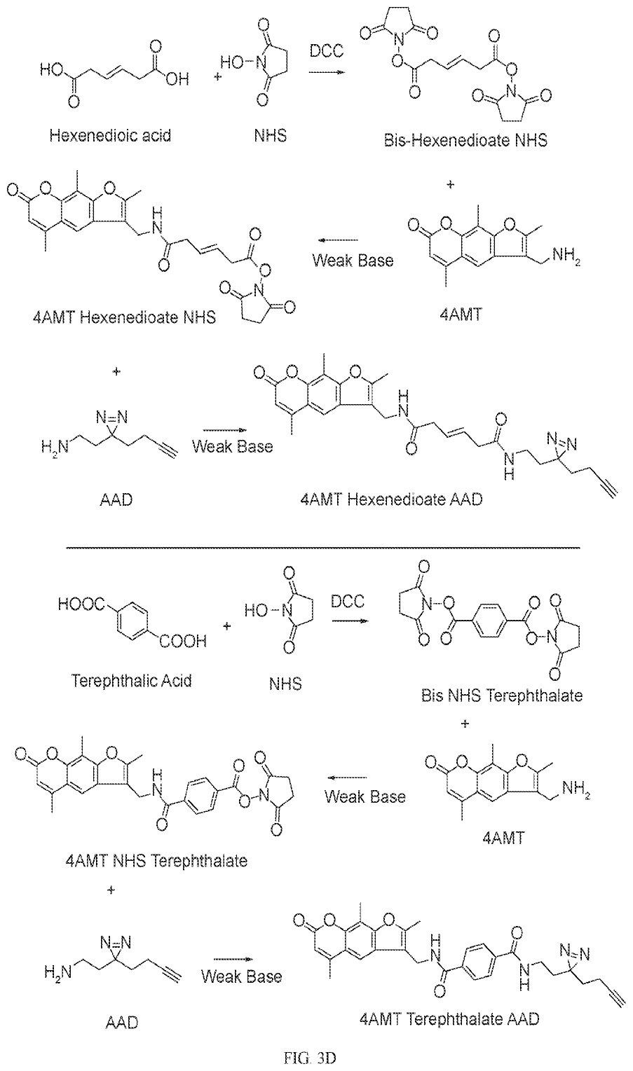

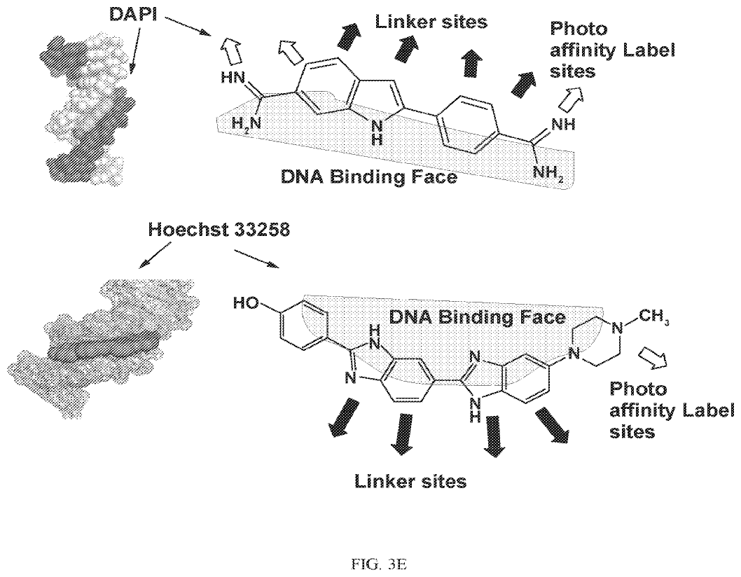

In various embodiments, the linker can comprise one or more of (i) a cleavable bond, (ii) an oligomer or polymer having a repeating unit of —OCH₂CH₂—, and (iii) an unsaturated moiety. Exemplary unsaturated moieties include but are not limited to carbon-carbon double bonds, triple bonds, and an aryl group (e.g., FIG. 3D).

The linker can also be designed to be cleavable so the proteins crosslinked to DNA complex could be released for mass spectrometry analyses. This is an important aspect in these embodiments, which will improve technologies that aim at capturing protein-bound DNA sequences (e.g. ChIP-seq and Hi-C), and open up a new field of mapping all proteins that are bound to DNA (DNA-bound proteome). One example of a probe with a cleavable linker is by coupling NHS-SS-diazirine (SDAD) with 4AMT to yield 4AMT-SS-SDAD. Similar to 4AMT-LC-SDA, 4AMT-SS-SDAD can be used to crosslink protein to DNA. But an added advantage of 4AMT-SS-SDAD is that upon purification of the crosslinked protein-DNA complexes, the proteins or their protease digested peptide fragments could be released by the cleavage of the linker to facilitate the subsequent protein analyses (e.g. by Mass Spectrometry). In addition to the disulfide linker described, other cleavable links that are stable in different cellular redox environments but cleavable under mild conditions could be used in the probe design (FIG. 3C). With these cleavable linker, BFPX probes could not only be used to detect DNA sequences bound by a given protein such as the ChIP-seq analyses, it will also enable genome-wide analyses of all proteins that were bound to DNA. In various embodiments, sulfoxide-containing MS-cleavable cross-linkers are used to introduce an MS-cleavable functional group into the linker for the BFPX probes; or the linker for the BFPX probes comprises sulfoxide-containing, MS-cleavable, C—S bonds. The two symmetric C—S bonds (introduced by these sulfoxide-containing MS-cleavable cross-linkers to the linker of the BFPX probes) can be preferentially cleaved in the gas phase using collision-induced dissociation (CID) during tandem mass spectrometry (MS/MS or MS²), for example by implementing higher-energy collisional dissociation (HCD) and/or electron transfer dissociation (ETD). This results in the physical separation of a nucleic acid-protein cross-link to yield unique peptide fragment pairs with a defined mass relationship. Exemplary sulfoxide-containing MS-cleavable cross-linkers for this use include DSSO (bis-(propionic acid NHS ester)-sulfoxide, Bis(2,5-dioxopyrrolidin-1-yl) 3,3′-sulfinyldipropionate), d0-DMDSSO (Bis(2,5-dioxopyrrolidin-1-yl) 3,3′-sulfinylbis(2-methylpropanoate)), DHSO (3,3′-Sulfinyldi(propanehydrazide); Dihydrazide sulfoxide), BMSO (3,3′-Sulfinylbis(N-(2-(2,5-dioxo-2,5-dihydro-1H-pyrrol-1-yl)ethyl)propanamide)), alkyne-A-DSBSO (Bis(2,5-dioxopyrrolidin-1-yl) 3,3′-((2-(but-3-yn-1-yl)-2-methyl-1,3-dioxane-5,5-diyl)bis(methylenesulfonyl))dipropionate; alkyne-tagged acid-cleavable disuccinimidyl bissulfoxide), and azide-A-DSBSO.

Compared with formaldehyde crosslinking, our design has the following advantages: (1) High specificity: Unlike formaldehyde which will react with any proteins in the cells (which will lead problems of epitope damage and excess fixing of cellular protein complexes, leading to difficulty in reversing crosslinked complexes), our probes will preferably be bound to DNA. After washing off the excess non-bound probes, the DNA bound probes will only be crosslinked to DNA and the nearby DNA-bound proteins; (2) Temporal and spatial selectivity: The crosslinking can be initiated by controlled UV illumination in terms of timing and focus, thus allowing for potential temporal and spatial control to capture protein/DNA complex in selected time and subcellular regions; (3) Because of the enhanced specific of crosslinking only proteins bound to DNA, in addition to identifying DNA sequences bound by proteins of interests (such as traditional ChIP-seq), it is also possible to use reversible linker, isotope labeling, and mass spectrometry to label all proteins that are bound to DNA throughout the genome upon UV illumination (conceptually an inverse of ChIP-seq, but to map DNA-bound proteome). This will provide unprecedented information for protein-DNA interactions at genome-wide and proteome-wide levels.

Various embodiments provide that the probes described in this invention are not limited to containing psoralen (or isopsoralen, or derivatives such as xanthotoxin/methoxsalen, bergapten, imperatorin, and nodakenetin) as the only nucleic acid-binding, photo-crosslinking group (FIG. 1A-FIG. 1E, FIG. 2A-FIG. 2C), and that the probes are not limited to binding towards double strand DNA only. Multiple other nucleic acid-binding molecules can be used to form the nucleic acid-binding head of a bifunctional probe of this invention, so long as they are equipped with a linkage group such as amine or azido. Their nucleic acids targets can be molecule specific.

In one embodiment, a nucleic acid-binding head of the bifunctional probes is derived from 4′,6-diamidino-2-phenylindole (DAPI), whose binding to dsDNA is genome wide non-specific and can be functionalized with an azido linkage group towards a click chemistry alkyne (FIG. 3A-FIG. 3E). DAPI will readily binds DNA solidly even without the need of photo chemistry.

In other embodiments, a nucleic acid-binding head of the bifunctional probes is a photo-reactive nucleic binder, which can be phenylazide, phenyl diazirine or benzophenone—all can bind DNA under similar spectrum illumination (300-360 nm)—and modified with attachment of amine/azido group as the other functional head for reaction with proteins (FIG. 3A-FIG. 3E).

Further embodiments provide that a nucleic acid-binding head of the bifunctional probes is a sequence specific binder, such as polyamides, which can be further modified to possess amine/azido linkage groups as the other functional head for reaction with proteins (FIG. 3A-FIG. 3E). Suitable nucleic acid-binding head/functional group is derived from pyrrole-imidazole polyamides, or hairpin polyamides containing N-methylpyrrole (Py), N-methylimidazole (Im), and N-methyl-3-hydroxypyrrole (Hp) residues, to bind specific predetermined sequences in nucleotides.

Among these, psoralen, phenylazide, phenyl diazirine or benzophenone polyamides can also be used for RNA binding, or single strand DNA binding.

Additionally, special molecule that recognizes specific nucleic acids targets such as G quartet recognition using multi-ring structure based anthracene or quinolinium derivatives can also be modified with an amino/azido linkage to a bifunctional probe to crosslink more efficiently with nearby biomolecules (FIG. 3A-FIG. 3C). Furthermore, Kethoxal (1,1-dihydroxy-3-ethoxy-2-butanone) that bind guanine at the N1 and N2 positions can be used as the nucleic acid binding head to develop BFPX probes targeting single strand DNA and RNA.

Additionally, using this design strategy the bifunctional probe can be further developed into having multiple other functions by a multi arm cyclooctyne core (e.g., dibenzocyclooctyne activated polyethylene glycol (PEG DBCO)) (FIG. 3B), such as for conjugating with a cleavable azido-diazo biotin (as enrichment handle), and/or with fluorophore azide to permit detection/identification of spatial location. The disclosed bifunctional/multi-functional probes conferring DNA with photocrosslinkable ends with an amine group and the ability to photocrosslink nearby protein via diazirine can on each end be modified into molecules with an azide group. When coupled with a multi-arm core, they can assemble in a customer-designed combinatorial way, such as on the cyclooctyne core by cooper-free bioorthogonal click chemistry, to be used in vivo or in vitro.

The photocrosslinking molecules (BFPX) provided herein are suitable for use in a wide range of genomics research tools such as ChIPmentation, Cut & Run, HiChIP, which traditionally depend on but are also limited by formaldehyde crosslinking.

We have developed a class of molecular probes that are cell permeable, inert to cellular molecules, bind DNA, and under illumination with long wavelength UVA (˜360 nm), become activated to covalently crosslink to DNA and proteins that bind to DNA nearby. These molecular probes can capture protein-nucleic acid complexes in vitro and in situ for a wide range of analyses at ensemble and single molecule level.

For a given cell at a certain state, what proteins bind to the genome and where they bind on the genomic DNA are fundamental questions to understanding cellular functions and disease mechanisms. Most protein-nucleic acid complexes are too unstable to be isolated from their native cellular environments due to their intrinsic, mostly ionic and hydrophilic interactions. A variety of techniques have been developed to capture protein-DNA complexes for biochemical and imaging analyses. Most of these methods depend on the capture of protein-DNA complexes by formaldehyde crosslinking.

However, increasing evidence suggests that formaldehyde fixation could be a major problem undermining the effectiveness of the current approaches.

This is largely due to the inefficiency of formaldehyde to crosslink DNA to proteins, and its highly reactive and non-specific damages to proteins, particularly at the DNA binding surfaces and antibody binding epitopes. The majority of DNA captured by formaldehyde is not covalently linked to protein but trapped in fixed protein complexes, leading to the capture of a large amount of non-specific DNA fragments. Direct UV crosslinking of protein to DNA and RNA have been reported, but these approaches are limited by the low crosslinking efficiency and the use of short wavelength UVC (˜250 nm) that damages proteins and nucleic acids. Here we have designed and synthesized a class of molecular probes that bind to (or intercalate) and enrich on DNA (or RNA) and can be activated by long wavelength UVA (˜360 nm) to form covalent linkages between DNA or RNA and their bound proteins. These molecular probes are biocompatible (inert to cellular molecules in the absence of UV), cell permeable, and upon UVA activation, can capture protein-DNA (or Protein-RNA) complexes inside cells with high specificity and stability.

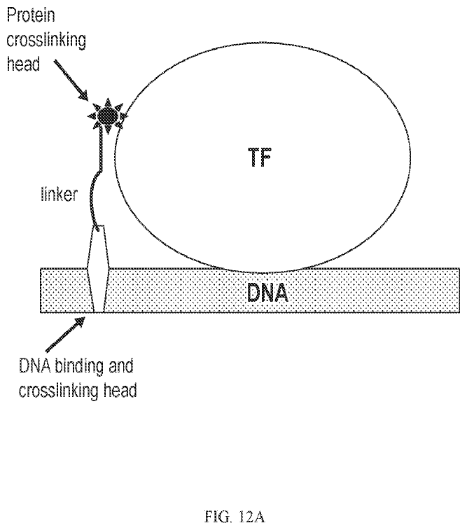

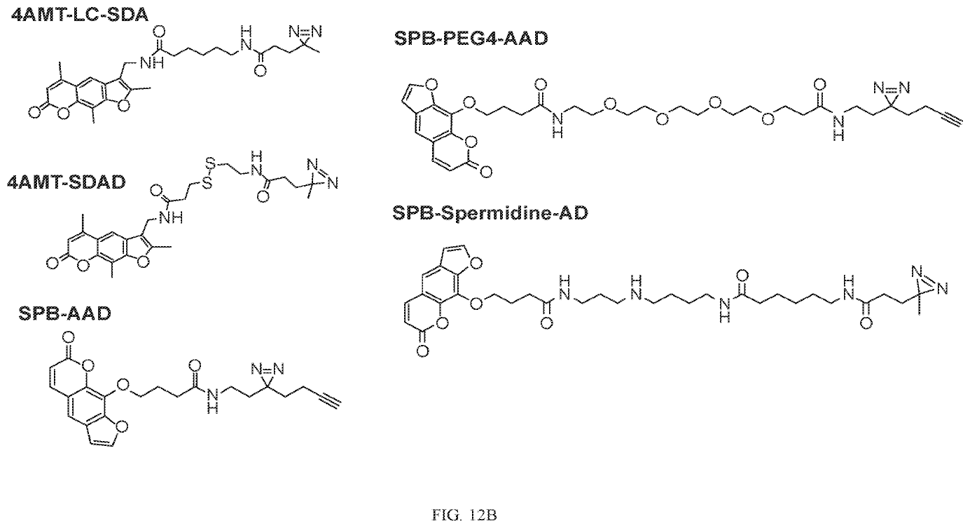

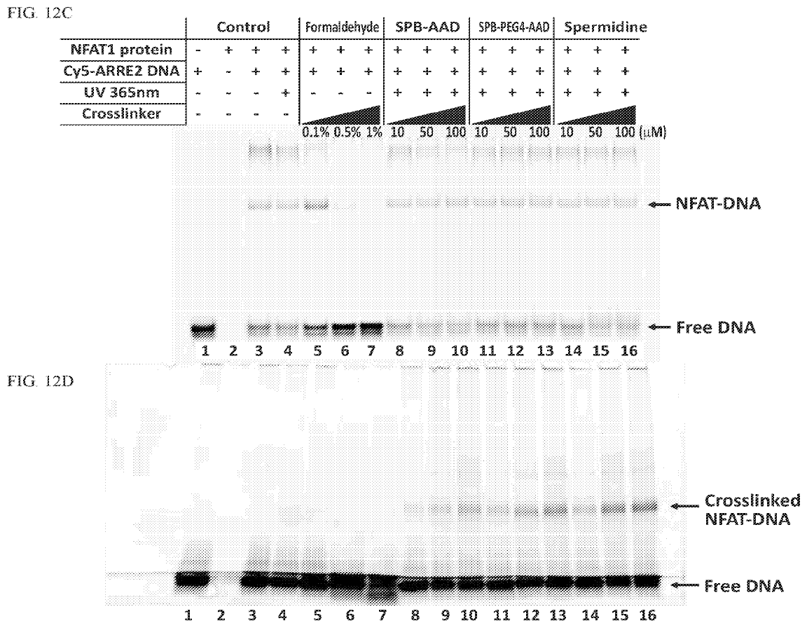

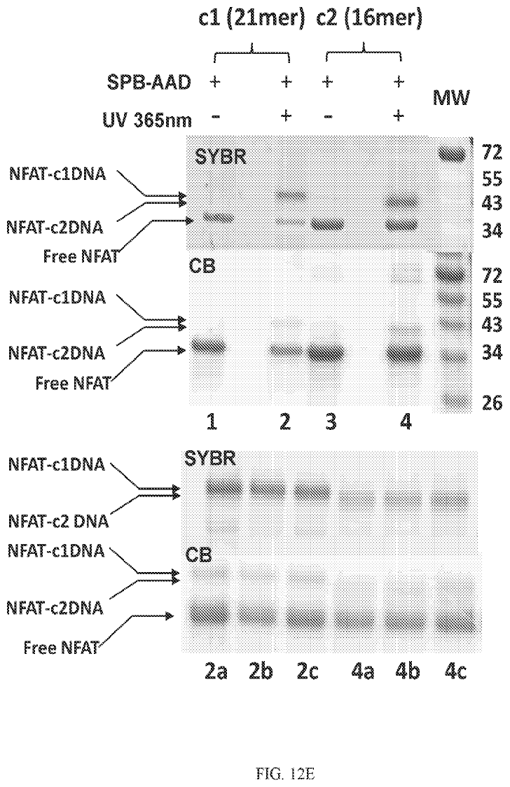

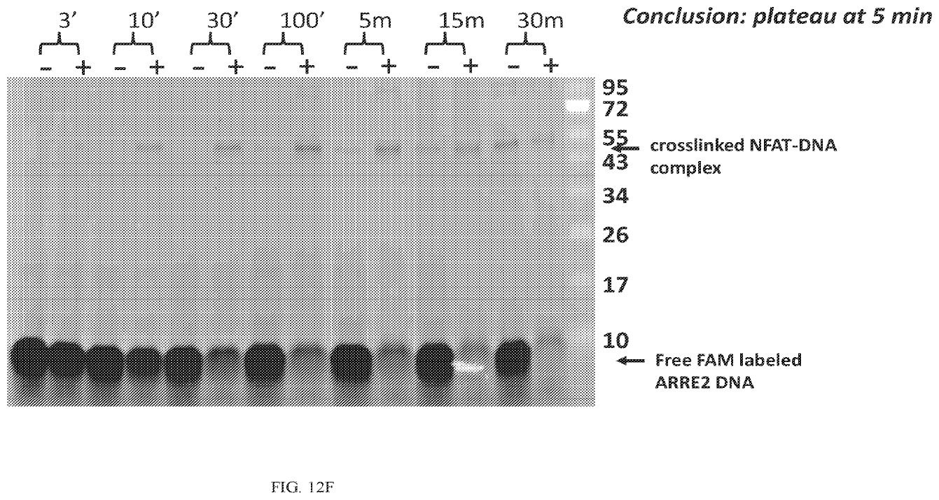

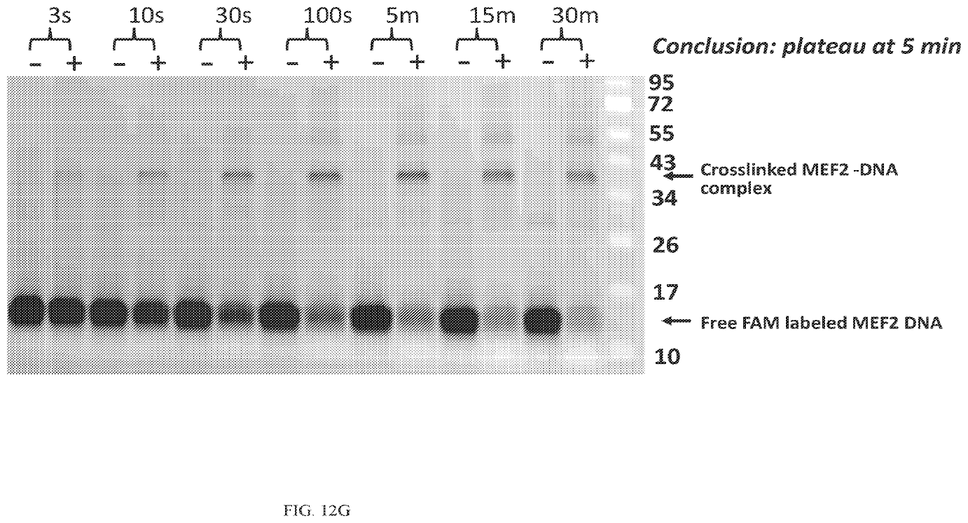

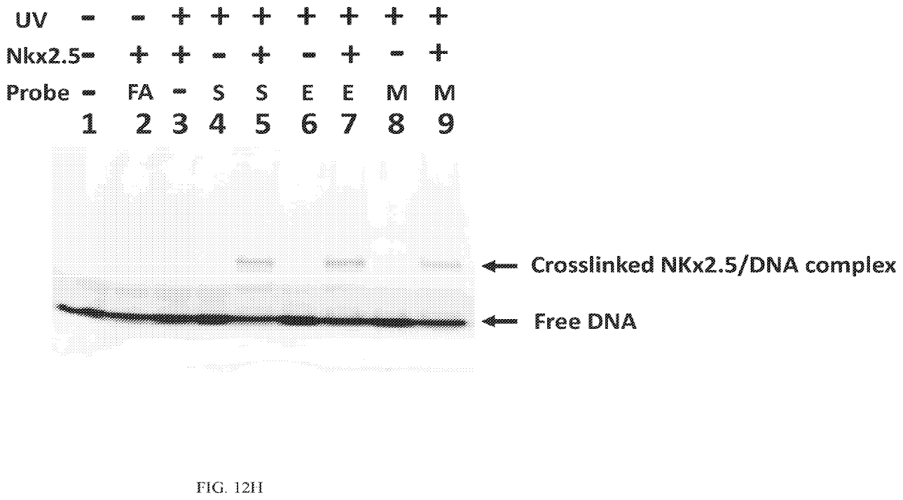

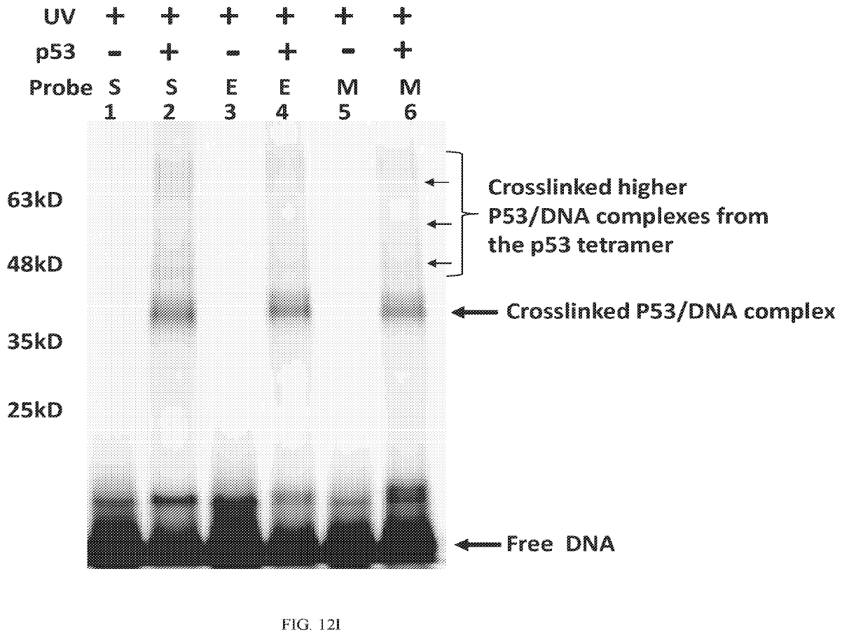

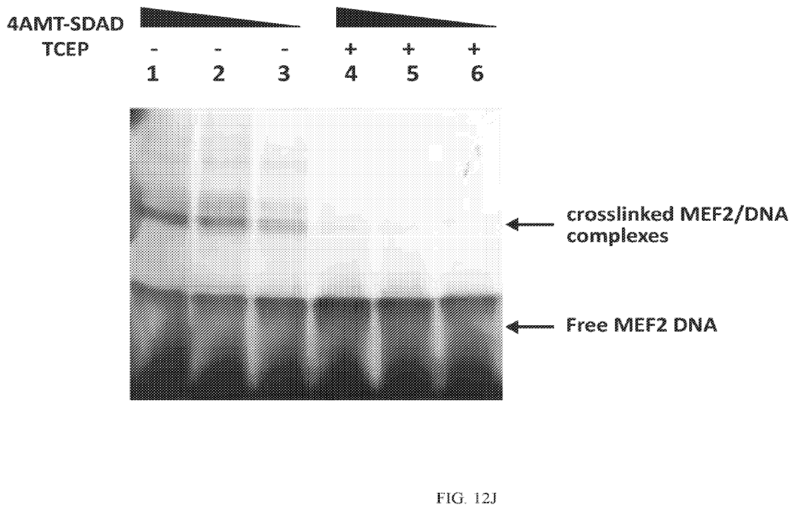

Our general strategy is to engineer molecular probes that have two different photo-crosslinking groups, hence the name bi-functional photo-crosslinking (BFPX) (FIG. 12A). One of the functional groups is responsible for binding and crosslinking to DNA or RNA under UVA illumination, and the other is to covalently capture proteins that are bound or recruited to DNA through UVA-activated photochemical reactions. The two functional groups are connected by a linker engineered to introduce molecular handles that can facilitate the monitoring/labelling, isolation and analyses of the crosslinked protein-DNA complexes.

For the DNA binding and crosslinking head, a number of natural or synthetic DNA binding molecules, including psoralen or derivatives, 4′,6-diamidino-2-phenylindole (DAPI), Hoechst dye that bind DNA nearly nonspecifically, could be used for genome-wide capture of any protein/DNA complexes. Alternatively, specific DNA binding molecules, such as sequence-specific DNA binding polyamides, or G quartet binders, could be used for targeted capture of protein-DNA complexes bound to specific genomic sites of interest. In the current studies, we choose the cell-permeable, non-toxic psoralen as the DNA anchoring head. Psoralen and derivatives can bind and intercalate DNA nearly non-specifically with a binding site preference of 5′-TA>5′-AT>>5′-TG>5′-GT′ and under long-wavelength (˜360 nm) UV illumination, crosslink to DNA covalently with high efficiency (up to 80%).

Psoralen has a modest affinity for double stranded DNA and RNA (Kd˜μM) leading to their enrichment on DNA without disrupting the DNA-binding of most transcription factors (Kd˜nM). For the protein capturing head, any photochemically activatable groups that form covalent linkage with proteins can be considered. In the current study we selected the well-established photo-affinity labelling group diazirine that has a similar long UVA wavelength activation spectrum (340-365 nm) to psoralen. The two heads are connected through a synthetic linkage of variable lengths, flexibility and cleavability. Following the above designing principles, we synthesized a series of BFPX probes with various linker lengths and functional features (FIG. 12B) (Examples section herein for details).

We first tested the activities of the BFPX probes using in vitro assembled transcription factor DNA complexes under both native (electrophoresis mobility shift assay, EMSA, FIG. 12C) and denaturing (SDS page, FIG. 12D) conditions.

As shown in FIG. 12C, UV alone (lane 4) and in combination with increasing concentration of BFPX probes, SPB-AAD (lanes 8-10), SPB-PEG4-AAD (lanes 11-13) and SPB-Spermidine-AD (lanes 14-16), showed little or no effect on the binding of NFAT1 (nuclear factor of activated T cells) to DNA as compared to the control (lane 3). By contrast, formaldehyde (lanes 5-7) showed a dosage dependent inhibition of the NFAT1/DNA binding interactions and at 1%, the typical concentration used in chromatin immunoprecipitation (ChIP) experiments, diminished most of the NFAT/DNA complexes (lane 7). Similar experiments with other transcription factors showed that the BFPX probes at a concentration up to 500 μM did not affect the DNA binding by GATA3 in EMSA whereas formaldehyde at as low as 0.1% (v/v) inhibited the DNA binding (data not shown).

When the same binding reactions of FIG. 12C were analyzed by denaturing SDS-PAGE (FIG. 12D), the native NFAT1/DNA complex dissociated as expected, (lane 3) and the formaldehyde crosslinking did not yield stable protein-DNA complexes (lanes 5-7), consistent with the notion that formaldehyde was not able to crosslink protein to DNA in vitro. By contrast, all three BFPX probes, SPB-AAD (lanes 8-10), SPB-PEG4-AAD (lanes 11-13) and SPB-Spermidine-AD (lanes 14-16), showed dosage dependent crosslinking of NFAT1/DNA complexes that are stable under strong denaturing conditions (2% SDS, 95° C. 10 min), suggesting the existence of stable covalent linkage between the protein and DNA. The crosslinked NFAT/DNA complexes can be well resolved by SDS gradient gel according to their expected molecular weight and can be stained by various methods, including cyanine dye (SYBR Safe) and Coomassie Blue (FIG. 12E).

SYBR Safe stained more effectively the Protein-DNA complex than the free protein whereas Coomassie Blue stain more effectively the free protein than the Protein-DNA complexes (FIG. 12E). Quantitative analysis of the crosslinking reaction is therefore best done using fluorescent labelled DNA. The apparent crosslinking efficiency (the intensity of the complex band over the total DNA intensity) can be as high as 22.5% (FIG. 12D, lane 16). When normalized against the total protein/DNA complex (i.e., only 48% of total DNA exist in the form of protein-DNA complex in lane 16, see FIG. 12C), the crosslinking efficiency is estimated to be 47% under this experimental condition. The BFPX crosslinking efficiency vary depending on the DNA affinity of the protein, the probes used, the binding conditions (salt and buffers), and the DNA sequences flanking the protein-binding site. The crosslinking reaction is also dosage dependent on UV illumination time (FIG. 12F, FIG. 12G). The crosslinked protein-DNA complexes were observed as early as 3 second, and the reaction almost reached plateau within 100 seconds. These observations raise the possibility of using BFPX to study the dynamic processes of protein-DNA interactions inside cells. We have demonstrated BFPX-based crosslinking of a wide range of transcription factor/DNA complexes that belong to distinct DNA binding domain families and bind DNA from the major or minor grooves. These including MEF2 (FIG. 12G), Nkx2.5 (FIG. 12H), and p53 (FIG. 12I) and GATA3 (not shown). Based on the molecular sizes, the crosslinked protein-DNA complexes contain one strand of the duplex DNA, indicating that the psoralen head of the BFPX probes form a mono adduct to DNA. The psoralen-DNA crosslink can be reversed by short UVC (254 nm) or alkaline heating. However, we found that these literature reported reversing procedures generate significant damages to protein and DNA (data not shown). One other way to release the crosslinked protein is to use a probe with a cleavable link as we demonstrated using 4AMT-SDAD in FIG. 12J. The BFPX-based crosslinking is strictly dependent on DNA binding, as large excess of proteins in the binding solution (such as BSA) that do not bind DNA are not crosslinked, and non-cognate DNA-binding proteins are not crosslinked when the DNA is bound by cognate proteins (data no shown). The specificity of the crosslinking reactions is further demonstrated at the structure level (see below).

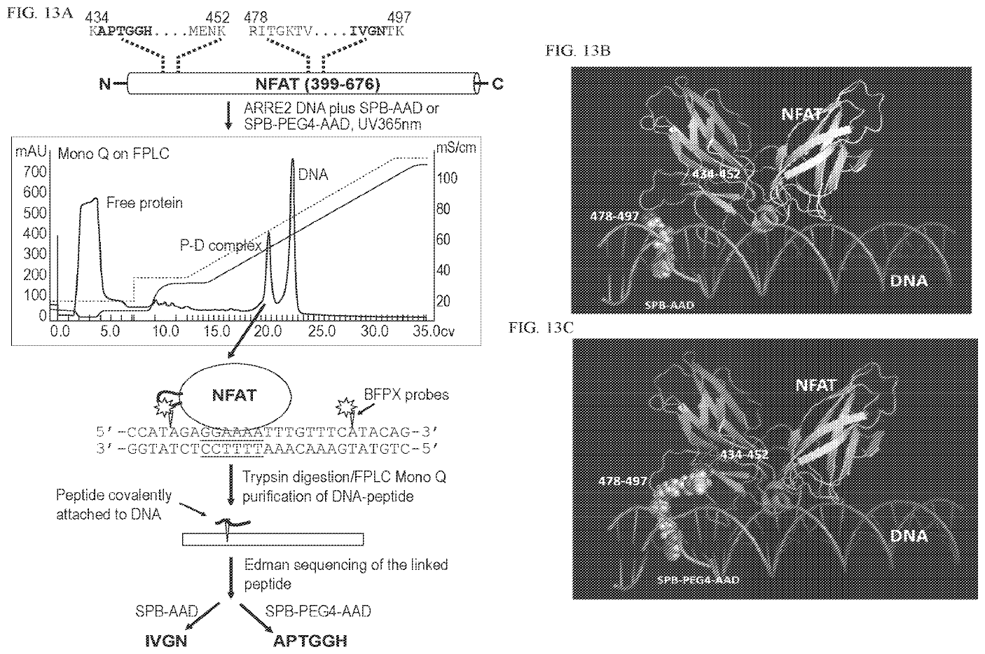

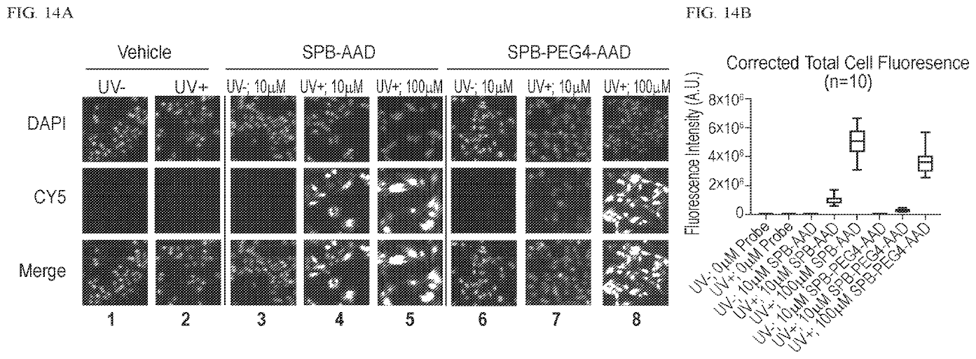

We analyzed the covalent attachment sites on the NFAT/DNA complexes crosslinked by SPB-AAD and SPB-PEG4-AAD using a work flow shown in FIG. 13A. The crosslinked NFAT/DNA complex was purified using a Mono-Q column on FPLC, digested by trypsin and purified by another round of Mono-Q on FPLC. The DNA with a peptide attached to it was eluted like free DNA. The DNA-peptide conjugate was then sequenced by Edman degradation. The sequencing results were generally noisy with multiple amino acids detected in each cycle. However, a major peptide could be identified each for the SPB-AAD and the SPB-PEG4-AAD crosslinked complexes, respectively. For the SPB-AAD crosslinked complex, the peptide (IVGN) matched uniquely to a tryptic fragment of NFAT1 between residues 478 and 497, while the peptide from the SPB-PEG4-AAD crosslinked complex (APTGGH) matched uniquely to a tryptic fragment between residues 434 and 452. These results are compared with the crystal structure of the NFAT/DNA complex (pdb code 1a02, Chen et al., Nature 1998). If we assume the BFPX probes inserts into 5′TpA-3′ sites on the DNA and that the linkers adopt an extended conformation, the diazirine head group of SPB-AAD would be adjacent to the loop (residues 478-497) of NFAT that is close to DNA (FIG. 13B), while the diazirine head group of SPB-PEG4-AAD would be adjacent to the loop (residues 434-452) of NFAT that is relatively further away from the DNA (FIG. 13C). These structure-based predictions matched very well with the Edman sequencing results. These analyses suggest strongly that the psoralen head in the BFPX probes still retain a preference for binding to the 5′-TpA-3′ site despite the synthetic modifications, and that the carbene generated through UV activation of diazirine favor crosslinking with proteins in a proximity dependent manner. These studies also demonstrate the utility of BFPX probes in structural analysis of protein-DNA interactions of both in vitro and in situ captured protein-DNA complexes.

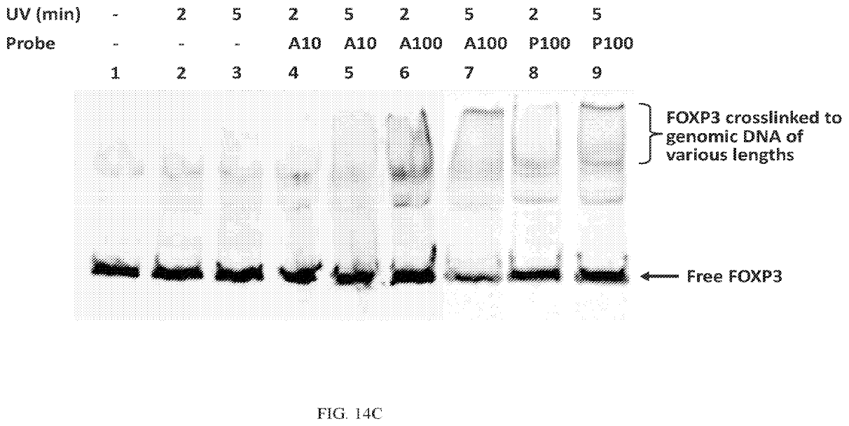

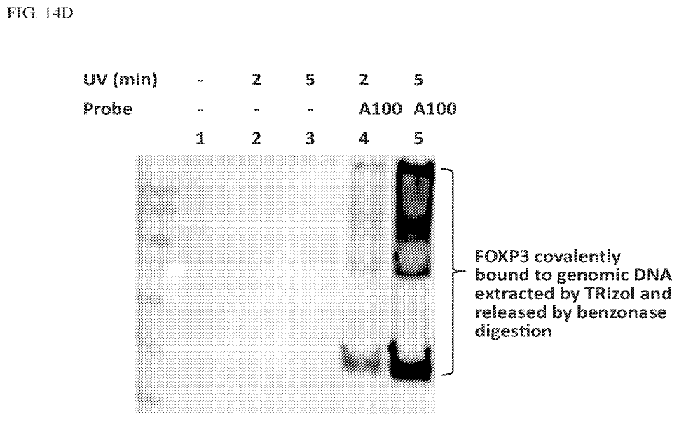

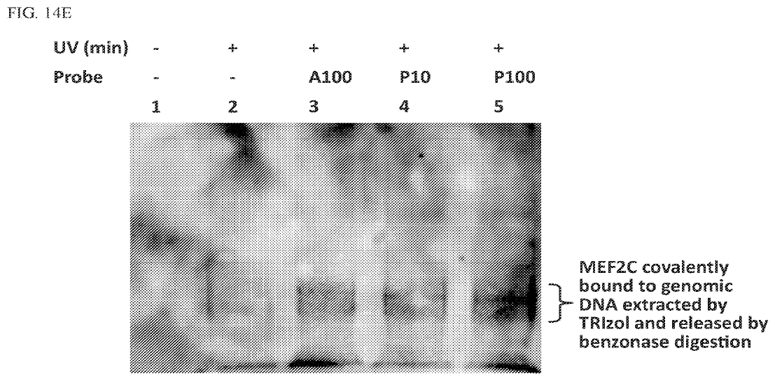

We next tested the cell and nuclear permeability of the BFPX probes using azide-containing fluorescent molecules through click reaction with the terminal alkyne in SPB-AAD and SPB-PEG4-AAD. Briefly, Hela cells were incubated with SPB-AAD or SPB-PEG4-AAD (10 μM or 100 μM) in the dark for 30 min to allow the DNA binding and intercalation by the psoralen moiety of the BFPX probes. After washing off the excess BFPX probes followed by illumination of UV 365 nm for 5 min, Alexa Fluor 647 picolyl azide was coupled to the alkyne group on SPB-AAD or SPB-PEG4-AAD via click reaction. After the click chemistry labeling, the unreacted fluorescent molecules were removed by washing. The nuclei were then be analyzed using fluorescence imaging (FIG. 14A). When no BFPX probes were added (columns 1 and 2) or adding probes without UV treatment (columns 3 and 6), little or no fluorescence signal was retained in the cells. By contrast, only when the BFPX probes were present and activated by UV crosslinking, the Alexa Fluor 647 picolyl azide molecule could be immobilized in the cells via click reaction with SPB-AAD (columns 4 and 5) or SPB-PEG4-AAD (columns 7 and 8) and the immobilized fluorescent signal is dosage dependent on the BFPX probes (FIG. 14B).

Finally, we tested the capture of transcription factors bound to genomic DNA inside cells. HEK293T cells transfected with FLAG-tagged FOXP3 were treated with various combination of UV illumination and probe (SPB-AAD: A; and P: SPB-PEG4-AAD). The cells were lysed in 1×RIPA buffer followed by RNase and MNase treatment. The whole cell lysate were then added SDS loading dye, heated at 95 degree C. and run on a 4-15% SDS PAGE gel and analyzed by western blot using an anti-FLAG antibody.

As shown in FIG. 14C, higher molecular weight species containing FOXP3 were observed in SPB-AAD (A) and SPB-PEG4-AAD (P) treated cells (lanes 4-9) as compared with controls (lanes 1-3), and the formation of crosslinked FOXP3 complexes is dosage dependent on probe concentration (compare lane 4 (SPB-AAD, 10 μM, A10), and lane 6 (SPB-AAD, 100 μM, A100)) and the UV exposure time (compare lane 4 (2 min) and lane 5 (5 min) at SPB-AAD concentration of 10 μM (A10); also compare lane 8 (2 min) and lane 9 (5 min) at SPB-PEG4 AAD concentration of 100 μM (P100). We also used TRIzol to extract genomic DNA from BFPX-treated and control cells. TRIzol uses strong denaturing reagent (e.g. 4 M guanidine thiocyanate) to lyse the cells and completely dissociate nucleoprotein complexes. The extracted genomic DNA was then digested extensively with Benzonase and analyzed by SDS PAGE gel and western blot. Using the same HEK293T cells transfected with FLAG-tagged FOXP3, as shown in FIG. 14D, it is clear that only cells treated with UV and probe (SPB-AAD, 100 μM, A100) showed FOXP3 proteins (lanes 4 and 5) as compared with the controls (lane 1-3). This observation again suggests that FOXP3 proteins were covalently linked to genomic DNA in BFPX treated cells that can be extracted by the TRIzol protocol under strong denaturing conditions whereas in control cells the non-covalent nucleoprotein complexes were indeed completely dissociated. Interestingly, Benzonase digestion releases not only FOXP3 with a molecular weight similar to its free monomer but also higher molecular species, suggesting that FOXP3 may bind some genomic region as higher order structures that are resistant to Benzonase digestion. We further test the capture of endogenous transcription factors in GM12878 using MEF2C as the target. GM12878 cells (7.5 million for each sample) were treated with different combinations of UV illumination and BFPX probe, and the genomic DNA was extracted using the TRIzol protocol, digested by Benzonase and analyzed by SDS-PAGE/western blot (FIG. 14E). Compared with the transfection system, the signal for endogenous transcription factors is substantially weaker due to the low natural abundance. Nevertheless, it is clear that MEF2C covalently bound to genomic DNA and extracted by TRizol under strong denaturing condition were only observed on cells treated with UV and probe (lanes, 3, 4 and 5) as compared with the control (lane 1). The UV only control (lane 2) showed a weak signal, suggesting that UV alone may crosslink some protein to DNA at a low level. But the crosslinking efficiency in BFPX-treated cells is significantly higher than the UV only control (compare lanes 3-5 and lane 2).

The BFPX strategy is mechanistically defined, its modular design enables the development of general and customized tools for capturing protein-nucleic acids complexes in vitro and inside cells for ensemble and single molecule analyses. Unlike formaldehyde and other lysine-reacting bi-functional chemical crosslinking probes, which will react with any proteins in the cells non-specially and uncontrollably, the BFPX probes will preferably bind only to nucleic acids by specific recognition heads, thereby achieving regioselectivity of photochemical reactions at or near the DNA binding site. The crosslinking can be initiated by UV illumination at specific time points and focused on specific subcellular locations, thus allowing for temporal and spatial resolved study of protein/nucleic acid interactions in situ. In addition to identifying DNA sequences bound by proteins of interests, it is also possible to use a reversible linker, isotope labeling, and mass spectrometry to identify all proteins that are bound to DNA throughout the genome at a specific time point. These new capabilities afforded by BFPX will provide unprecedented information for protein-DNA interactions at genome-wide and proteome-wide scales.

### VARIOUS EMBODIMENTS OF THE INVENTION

Embodiments include those listed below.

Embodiment 1. A photocrosslinking molecule of Formula (I): A-L1-(C)ₙ-((L2)ₙ-B)ₘ, (I), wherein A represents a nucleic acid-binding functional group derived from psoralen, methyltrioxsalen, benzophenone, 4′,6-diamidino-2-phenylindole (DAPI), a Hoechst dye, a polyamide, or a G quartet binding molecule, or a derivative thereof; L1 is absent or represents a first linker; C represents, when n=1, a core moiety having at least two functional groups each separately for attachment to L1 and attachment to at least one arm represented by L2-B; or C is absent when n=0; L2 represents, when n=1, independently a second linker for each arm represented by L2-B; or L2 is absent when n=0; B represents independently for said each arm: a photo-reactive functional group comprising diazirine or its derivative or an aryl azide or its derivative, optionally the aryl azide or its derivative selected from phenyl azide, orthro-hydroxyphenyl azide, meta-hydroxyphenyl azide, tetrafluorophenyl azide, ortho-nitropenyl azide, meta-nitropenyl azide, or azdo-methylcoumarin; or a detectable functional group; wherein in at least one said each arm, B represents the photo-reactive functional group; n=0 or 1; m represents number of arms represented by (L2)ₙ-B, wherein m is an integer being 1 or greater when n=1, or m=1 when n=0.

Embodiment 2. The photocrosslinking molecule of embodiment 1, wherein at least one of L1 and L2 is not absent, and the at least one of L1 and L2 is cleavable.

Embodiment 3. The photocrosslinking molecule of embodiment 2, wherein L1, L2, or both independently comprise one or more of a sulfoxide-containing mass spectrometry (MS)-cleavable bond, an acid-cleavable C—S bond, a disulfide group, and an azo group.

Embodiment 4. The photocrosslinking molecule of any one of embodiments 1-3, wherein n=0, m=1, and the photocrosslinking molecule is represented by formula (II): A-L1-B (11).

Embodiment 5. The photocrosslinking molecule of embodiment 4, wherein: A is an amine-containing or amine-reactive derivative of the psoralen, the methyltrioxsalen, the benzophenone, the DAPI, the Hoechst dye, the polyamide, or the G quartet binding molecule, optionally A being derived from succinimidyl-[4-(psoralen-8-yloxy)]-butyrate (SPB) or 4′-aminomethyltrioxsalen (4AMT); B comprises a diazirine or a diazirine alkyne, optionally an amino diazirine alkyne (AAD); and L1 is absent or the first linker, wherein the first linker comprises one or more of (i) a cleavable bond, (ii) an oligomer or polymer having a repeating unit of —OCH₂CH₂- and (iii) an unsaturated moiety, optionally selected from a carbon-carbon double bond, a carbon-carbon triple bond, or an aryl group.

Embodiment 6. The photocrosslinking molecule of embodiment 5, wherein L1-B is derived from succinimidyl 6-(4,4′-azipentanamido)hexanoate (NHS-LC-SDA), succinimidyl 2-((4,4′-azipentanamido)ethyl)-1,3′dithiopropionate (NHS-SS-Diazirine), or 2-(3-(But-3-yn-1-yl)-3H-diazirin-3-yl)ethan-1-amine (AAD); and/or wherein A is derived from 4′-aminomethyltrioxsalen (4AMT) or succinimidyl-[4-(psoralen-8-yloxy)]-butyrate (SPB); and wherein optionally the photocrosslinking molecule is represented by formula (IIa) or formula (IIc):

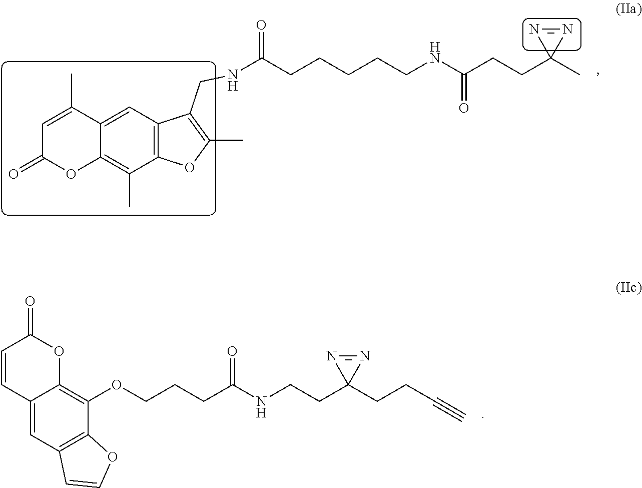 [SMILES: Cc1oc2c(C)c3oc(=O)cc(C)c3cc2c1CNC(=O)CCCCCNC(=O)CCC1(C)N=N1 ; C#CCCC1(CCNC(=O)CCCOc2c3occc3cc3ccc(=O)oc23)N=N1]

Embodiment 7. The photocrosslinking molecule of embodiment 5, wherein: A is derived from SPB or 4AMT; B comprises a diazirine or a diazirine alkyne, optionally an amino diazirine alkyne (AAD); and L1 is the first linker comprising one or more of (i) a cleavable bond, (ii) an oligomer or polymer having a repeating unit of —OCH₂CH₂—, and (iii) an unsaturated moiety, said unsaturated moiety optionally selected from a carbon-carbon double bond or an aryl group; and wherein optionally the photocrosslinking molecule is represented by formula (IIb), (IId), (IIe), or (IIf):

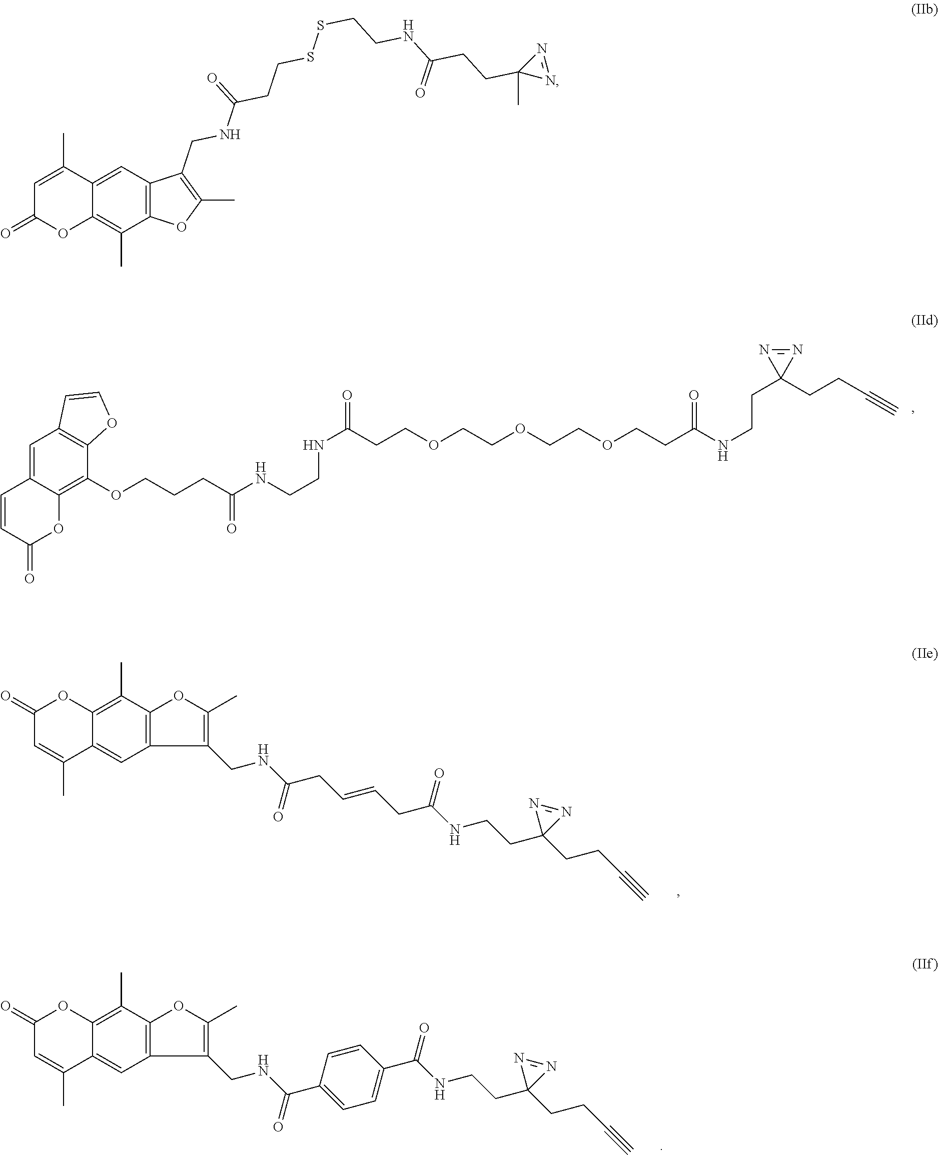

Embodiment 8. The photocrosslinking molecule of embodiment 5, wherein L1 comprises 2 to 20 carbons or 20-100 carbons in length.

Embodiment 9. The photocrosslinking molecule of any one of embodiments 1-3, wherein n=1, m is an integer being 2 or greater, and C represents a core moiety having at least three functional groups each separately for attachment to L1 and attachment to the at least two arms each represented by (L2-B), so that the photocrosslinking molecule is represented by Formula (III):

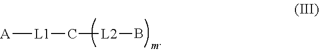

Embodiment 10. The photocrosslinking molecule of embodiment 9, wherein B comprises diazirine or an azide diazirine in one of the at least two arms, and B represents a detectable functional group in another one of the at least two arms, said detectable function group comprising a fluorophore, a biotin, a chromophore, a chromogen, a quantum dot, a fluorescent microsphere, or a nanoparticle.

Embodiment 11. The photocrosslinking molecule of embodiment 9 or 10, wherein L1, L2, or both independently comprise one or more of (i) a cleavable bond, (ii) an oligomer or polymer having a repeating unit of —OCH₂CH₂—, and (iii) an unsaturated moiety.

Embodiment 12. The photocrosslinking molecule of any one of embodiments 9-11, wherein C represents a dendritic core moiety comprising at least three surface functional groups each separately for attachment to L1 and attachment to the at least two arms each represented by L2-B.

Embodiment 13. The photocrosslinking molecule of any one of embodiments 9-12, wherein L1, L2, or both independently comprise a triazole in bonding with A.

Embodiment 14. A method of crosslinking a nucleic acid with a protein in proximity in a system, comprising: incubating the photocrosslinking molecule of any one of embodiments 1-13 with the system, and irradiating the system with an ultraviolet light.

Embodiment 15. The method of embodiment 14, wherein the system is a live cell.

Embodiment 16. The method of embodiment 14 or 15, wherein the ultraviolet light is between 300 nm and 360 nm in wavelength.

Embodiment 17. The method of any one of embodiments 14-16, further comprising performing one or more of immuno precipitation, chromatic precipitation, 3D chromatin conformation capture, mass spectrometry, and electrophoresis, with the system.

Embodiment 18. The method of any one of embodiments 14-17, wherein element L1, L2, or both of the photocrosslinking molecule is independently cleavable, and the method further comprises adding a cleaving agent to the system to cleave the elements L1, L2, or both; or wherein element A of the photocrosslinking molecule is derived from psoralen, and the method further comprises applying an ultraviolet light of about 230 nm in wavelength to cleave the element A; thereby generating a fingerprint of crosslinked proteins in proximity to nucleic acids in the system.

Embodiment 19. A method for preparing the photocrosslinking molecule of any one of embodiments 9-13, comprising: providing an azide derivative of a nucleic acid-binding, photo-reactive agent comprising psoralen, methyltrioxsalen, benzophenone, 4′,6-diamidino-2-phenylindole (DAPI), a Hoechst dye, a polyamide, or a G quartet binding molecule, or a derivative thereof, providing an azide derivative of a photo-reactive agent that comprises a diazirine moiety so as to obtain an azide-diazirine bifunctional, photo-reactive agent, and said photo-reactive agent optionally further comprising an alkyne group, or providing an aryl azide, said aryl azide optionally selected from phenyl azide, orthro-hydroxyphenyl azide, meta-hydroxyphenyl azide, tetrafluorophenyl azide, ortho-nitropenyl azide, meta-nitropenyl azide or azdo-methylcoumarin; optionally providing an azide derivative of a detectable agent comprising a fluorophore, a biotin, a chromophore, a chromogen, a quantum dot, a fluorescent microsphere, or a nanoparticle; providing a multi-arm agent having at least three functional groups each independently comprising an alkyne; and combining each azide derivatives and, if provided, the aryl azide, together with the multi-arm agent in one reaction vessel to prepare the photocrosslinking molecule.

Embodiment 20. The method of embodiment 19, wherein the multi-arm agent has at least three functional groups each independently comprising a cyclooctyne group.

Embodiment 21. The method of embodiment 19 or 20, wherein the nucleic acid-binding, photo-reactive agent comprises a first primary amine functional group, and providing the azide derivative of the nucleic acid-binding, photo-reactive agent comprises converting the first primary amine functional group to a first azide-containing moiety, optionally via reacting the nucleic acid-binding, photo-reactive agent with imidazole-1-sulfonyl azide; and/or wherein the photo-reactive agent that comprises a diazirine moiety further comprises a second primary amine functional group or is modified with the second primary amino functional group, and providing the azide derivative of said photo-reactive agent comprises converting the second primary amine functional group to a second azide-containing moiety, optionally via reacting said photo-reactive agent with imidazole-1-sulfonyl azide.

Additional embodiments include those listed below.

In some embodiments, L1 comprises 2 to 20 carbons or 20-100 carbons in length. In some embodiments, L1 comprises 2 to 20 carbons, 2 to 19 carbons, 2 to 18 carbons, 2 to 17 carbons, 2 to 16 carbons, 2 to 15 carbons, 2 to 14 carbons, 2 to 13 carbons, 2 to 12 carbons, 2 to 11 carbons, 2 to 10 carbons, 2 to 9 carbons, 2 to 8 carbons, 2 to 7 carbons, 2 to 6 carbons, 2 to 5 carbons, 2 to 4 carbons, 2 to 3 carbons.

In some embodiments, L1 comprises 20-100 carbons in length, 20-95 carbons in length, 20-90 carbons in length, 20-85 carbons in length, 20-80 carbons in length, 20-75 carbons in length, 20-70 carbons in length, 20-65 carbons in length, 20-60 carbons in length, 20-55 carbons in length, 20-50 carbons in length, 20-45 carbons in length, 20-40 carbons in length, 20-35 carbons in length, 20-30 carbons in length, or 20-25 carbons in length.

In some embodiments, L2 comprises 2 to 20 carbons or 20-100 carbons in length. In some embodiments, L2 comprises 2 to 20 carbons, 2 to 19 carbons, 2 to 18 carbons, 2 to 17 carbons, 2 to 16 carbons, 2 to 15 carbons, 2 to 14 carbons, 2 to 13 carbons, 2 to 12 carbons, 2 to 11 carbons, 2 to 10 carbons, 2 to 9 carbons, 2 to 8 carbons, 2 to 7 carbons, 2 to 6 carbons, 2 to 5 carbons, 2 to 4 carbons, 2 to 3 carbons.

In some embodiments, L2 comprises 20-100 carbons in length, 20-95 carbons in length, 20-90 carbons in length, 20-85 carbons in length, 20-80 carbons in length, 20-75 carbons in length, 20-70 carbons in length, 20-65 carbons in length, 20-60 carbons in length, 20-55 carbons in length, 20-50 carbons in length, 20-45 carbons in length, 20-40 carbons in length, 20-35 carbons in length, 20-30 carbons in length, or 20-25 carbons in length.

In some embodiments, the detectable functional group comprises a fluorophore, a biotin, a chromophore, a chromogen, a quantum dot, a fluorescent microsphere, or a nanoparticle. In some embodiments, the detectable functional group comprises a fluorophore. In some embodiments, the detectable functional group comprises a biotin. In some embodiments, the detectable functional group comprises a chromophore. In some embodiments, the detectable functional group comprises a chromogen. In some embodiments, the detectable functional group comprises a quantum dot. In some embodiments, the detectable functional group comprises a fluorescent microsphere. In some embodiments, the detectable functional group comprises a nanoparticle.

In some embodiments, the detectable functional group is a fluorophore, a biotin, a chromophore, a chromogen, a quantum dot, a fluorescent microsphere, or a nanoparticle. In some embodiments, the detectable functional group is a fluorophore. In some embodiments, the detectable functional group is a biotin. In some embodiments, the detectable functional group is a chromophore. In some embodiments, the detectable functional group is a chromogen. In some embodiments, the detectable functional group is a quantum dot. In some embodiments, the detectable functional group is a fluorescent microsphere. In some embodiments, the detectable functional group is a nanoparticle.

In some embodiments a compound of the present invention is a photocrosslinking molecule. In some embodiments, a compound of Formula (I) is a photocrosslinking molecule. In some embodiments, a compound of Formula (II) is a photocrosslinking molecule. In some embodiments, a compound of Formula (III) is a photocrosslinking molecule. In some embodiments, a compound of Formula (IIa) is a photocrosslinking molecule. In some embodiments, a compound of Formula (IIb) is a photocrosslinking molecule. In some embodiments, a compound of Formula (IIc) is a photocrosslinking molecule. In some embodiments, a compound of Formula (IId) is a photocrosslinking molecule. In some embodiments, a compound of Formula (IIe) is a photocrosslinking molecule.

In some embodiments a compound of the present invention is a bi-functional photo-crosslinking (BFPX) probe. In some embodiments, a compound of Formula (I) is a bi-functional photo-crosslinking (BFPX) probe. In some embodiments, a compound of Formula (II) is a bi-functional photo-crosslinking (BFPX) probe. In some embodiments, a compound of Formula (III) is a bi-functional photo-crosslinking (BFPX) probe. In some embodiments, a compound of Formula (IIa) is a bi-functional photo-crosslinking (BFPX) probe. In some embodiments, a compound of Formula (IIb) is a bi-functional photo-crosslinking (BFPX) probe. In some embodiments, a compound of Formula (IIc) is a bi-functional photo-crosslinking (BFPX) probe. In some embodiments, a compound of Formula (IId) is a bi-functional photo-crosslinking (BFPX) probe. In some embodiments, a compound of Formula (IIe) is a bi-functional photo-crosslinking (BFPX) probe.

In some embodiments, a compound of Formula (II) is a compound of Formula (I). In some embodiments, a compound of Formula (III) is a compound of Formula (I). In some embodiments, a compound of Formula (IIa) is a compound of Formula (II). In some embodiments, a compound of Formula (IIb) is a compound of Formula (II). In some embodiments, a compound of Formula (IIc) is a compound of Formula (II). In some embodiments, a compound of Formula (IId) is a compound of Formula (II). In some embodiments, a compound of Formula (IIe) is a compound of Formula (II). In some embodiments, a compound of Formula (Ha) is a compound of Formula (I). In some embodiments, a compound of Formula (IIb) is a compound of Formula (I). In some embodiments, a compound of Formula (IIc) is a compound of Formula (I). In some embodiments, a compound of Formula (IId) is a compound of Formula (I). In some embodiments, a compound of Formula (IIe) is a compound of Formula (I).

In some embodiments, a compound of Formula (I) is: A-L1-B. In some embodiments, a compound of Formula (I) is: A-B. In some embodiments, a compound of Formula (II) is: A-L1-B. In some embodiments, a compound of Formula (II) is: A-B.

In some embodiments, the ultraviolet light comprises UVA light, UVB light, or UVC light, or combination thereof. In some embodiments, the ultraviolet light comprises UVA and UVB light. In some embodiments, the ultraviolet light comprises UVB and UVC light. In some embodiments, the ultraviolet light comprises UVA and UVC light. In some embodiments, the ultraviolet light comprises only UVA light. In some embodiments, the ultraviolet light comprises only UVB light. In some embodiments, the ultraviolet light comprises only UVC light.

In some embodiments, the ultraviolet light is UVA light, UVB light, or UVC light, or combination thereof. In some embodiments, the ultraviolet light is UVA and UVB light. In some embodiments, the ultraviolet light is UVB and UVC light. In some embodiments, the ultraviolet light is UVA and UVC light. In some embodiments, the ultraviolet light is only UVA light. In some embodiments, the ultraviolet light is only UVB light. In some embodiments, the ultraviolet light is only UVC light.

Without being bound by theory, in some embodiments the ultraviolet light is 100 nm to 400 nm in wavelength. Without being bound by theory, in some embodiments the UVA light is 315 nm to 400 nm in wavelength. Without being bound by theory, in some embodiments the UVB light is 280 nm to 315 nm in wavelength. Without being bound by theory, in some embodiments the UVC light is 100 nm to 280 nm in wavelength.

Without being bound by theory, in some embodiments the ultraviolet light is 100 nm to 400 nm in wavelength. Without being bound by theory, in some embodiments the UVA light is 315 nm to 400 nm in wavelength. Without being bound by theory, in some embodiments the UVB light is 280 nm to 314 nm in wavelength. Without being bound by theory, in some embodiments the UVC light is 100 nm to 279 nm in wavelength.

In some embodiments, the ultraviolet light is between 300 nm and 360 nm in wavelength. In some embodiments, the ultraviolet light is 300 nm to 360 nm in wavelength. In some embodiments, the ultraviolet light is 300 nm to 400 nm in wavelength, 300 nm to 310 nm in wavelength, 300 nm to 320 nm in wavelength, 300 nm to 330 nm in wavelength, 300 nm to 340 nm in wavelength, 300 nm to 350 nm in wavelength, 300 nm to 360 nm in wavelength, 300 nm to 370 nm in wavelength, 300 nm to 379 nm in wavelength, 300 nm to 380 nm in wavelength, or 300 nm to 390 nm in wavelength.

In some embodiments, the ultraviolet light is 400 nm to 390 nm in wavelength, 400 nm to 380 nm in wavelength, 400 nm to 370 nm in wavelength, 400 nm to 360 nm in wavelength, 400 nm to 350 nm in wavelength, 400 nm to 340 nm in wavelength, 400 nm to 330 nm in wavelength, 400 nm to 320 nm in wavelength, 400 nm to 316 nm in wavelength, 400 nm to 315 nm in wavelength, 400 nm to 310 nm in wavelength, or 400 nm to 300 nm in wavelength.

In some embodiments, the ultraviolet light is 315 nm to 400 nm in wavelength, 315 nm to 390 nm in wavelength, 315 nm to 380 nm in wavelength, 315 nm to 370 nm in wavelength, 315 nm to 360 nm in wavelength, 315 nm to 350 nm in wavelength, 315 nm to 340 nm in wavelength, or 315 nm to 330 nm in wavelength.

In some embodiments, the ultraviolet light is 316 nm to 400 nm in wavelength, 316 nm to 390 nm in wavelength, 316 nm to 380 nm in wavelength, 316 nm to 379 nm in wavelength, 316 nm to 370 nm in wavelength, 316 nm to 360 nm in wavelength, 316 nm to 350 nm in wavelength, 316 nm to 340 nm in wavelength, or 316 nm to 330 nm in wavelength. In some embodiments, the ultraviolet light is 316 nm to 379 nm in wavelength. In some embodiments, the nucleic acid comprises deoxyribonucleic acid (DNA), or ribonucleic acid (RNA), or combination thereof. In some embodiments, the nucleic acid comprises deoxyribonucleic acid (DNA). In some embodiments, the nucleic acid comprises ribonucleic acid (RNA).

In some embodiments, the nucleic acid is deoxyribonucleic acid (DNA), or ribonucleic acid (RNA), or combination thereof. In some embodiments, the nucleic acid is deoxyribonucleic acid (DNA). In some embodiments, the nucleic acid is ribonucleic acid (RNA).

Additional embodiments include those listed below.

Embodiment 1A. A compound of Formula (I):

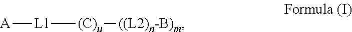

wherein:
A represents a nucleic acid-binding functional group derived from psoralen, methyltrioxsalen, benzophenone, 4′,6-diamidino-2-phenylindole (DAPI), a Hoechst dye, a polyamide, or a G quartet binding molecule, kethoxal, or a derivative thereof;
L1 is absent or represents a first linker;
C represents, when n=1, a core moiety having at least two functional groups each separately for attachment to L1 and attachment to at least one arm represented by L2-B; or C is absent when n=0;
L2 represents, when n=1, independently a second linker for each arm represented by L2-B; or L2 is absent when n=0;
B represents independently for said each arm:
   a photo-reactive functional group comprising diazirine or its derivative or an aryl azide or its derivative, optionally the aryl azide or its derivative selected from phenyl azide, orthro-hydroxyphenyl azide, meta-hydroxyphenyl azide, tetrafluorophenyl azide, ortho-nitropenyl azide, meta-nitropenyl azide, or azdo-methylcoumarin; or
   a detectable functional group;
   wherein in at least one said each arm, B represents the photo-reactive functional group;
n=0 or 1;
m represents number of arms represented by (L2)ₙ-B, wherein m is an integer being 1 or
greater when n=1, or m=1 when n=0.

Embodiment 2A. The compound of embodiment 1A, wherein at least one of L1 and L2 is not absent, and the at least one of L1 and L2 is cleavable.

Embodiment 3A. The compound of embodiment 2A, wherein L1, L2, or both independently comprise one or more of a sulfoxide-containing mass spectrometry (MS)-cleavable bond, an acid-cleavable C—S bond, a disulfide group, and an azo group.

Embodiment 4A. The compound of any one of embodiments 1A-3A, wherein n=0, m=1, and the compound is represented by Formula (II):

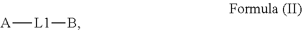

wherein L1 is absent or the first linker.

Embodiment 5A. The compound of embodiment 4A, wherein:
A is an amine-containing or amine-reactive derivative of the psoralen, an amine-containing or amine-reactive derivative of the methyltrioxsalen, an amine-containing or amine-reactive derivative of the benzophenone, an amine-containing or amine-reactive derivative of the 4′,6-diamidino-2-phenylindole (DAPI), an amine-containing or amine-reactive derivative of the Hoechst dye, an amine-containing or amine-reactive derivative of the polyamide, or an amine-containing or amine-reactive derivative of the G quartet binding molecule, or an amine-containing or amine-reactive derivative of kethoxal, optionally A being derived from succinimidyl-[4-(psoralen-8-yloxy)]-butyrate (SPB) or 4′-aminomethyltrioxsalen (4AMT);
B comprises a diazirine or a diazirine alkyne, optionally an amino diazirine alkyne (AAD); and
L1 is absent or the first linker, wherein the first linker comprises one or more of (i) a cleavable bond, (ii) an oligomer or polymer having a repeating unit of —OCH₂CH₂—, and (iii) an unsaturated moiety, optionally selected from a carbon-carbon double bond, a carbon-carbon triple bond, or an aryl group.

Embodiment 6A. The compound of embodiment 5A, wherein L1-B is derived from succinimidyl 6-(4,4′-azipentanamido)hexanoate (NHS-LC-SDA), succinimidyl 2-((4,4′-azipentanamido)ethyl)-1,3′dithiopropionate (NHS-SS-Diazirine), or 2-(3-(But-3-yn-1-yl)-3H-diazirin-3-yl)ethan-1-amine (AAD); and/or wherein A is derived from 4′-aminomethyltrioxsalen (4AMT) or succinimidyl-[4-(psoralen-8-yloxy)]-butyrate (SPB); and wherein optionally the photocrosslinking molecule is represented by Formula (IIa) or Formula (IIc):

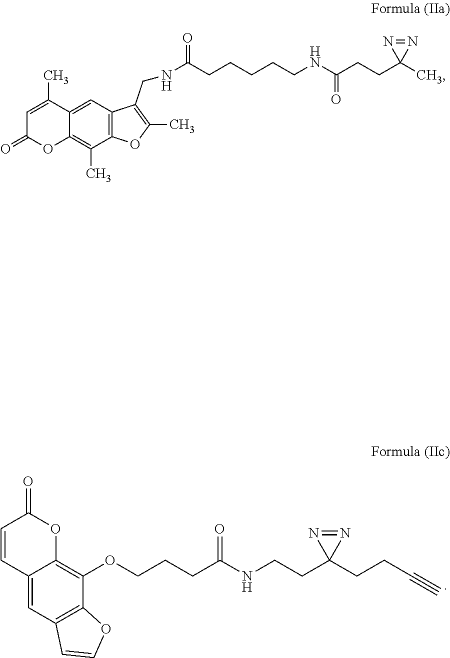 [SMILES: Cc1oc2c(C)c3oc(=O)cc(C)c3cc2c1CNC(=O)CCCCCNC(=O)CCC1(C)N=N1 ; C#CCCC1(CCNC(=O)CCCOc2c3occc3cc3ccc(=O)oc23)N=N1]

Embodiment 7A. The compound of embodiment 5A, wherein:
A is derived from succinimidyl-[4-(psoralen-8-yloxy)]-butyrate (SPB) or 4′-aminomethyltrioxsalen (4AMT);
B comprises a diazirine or a diazirine alkyne, optionally an amino diazirine alkyne (AAD); and
L1 is the first linker comprising one or more of (i) a cleavable bond, (ii) an oligomer or polymer having a repeating unit of —OCH₂CH₂—, and/or (iii) an unsaturated moiety, said unsaturated moiety optionally selected from a carbon-carbon double bond or an aryl group; and wherein optionally the photocrosslinking molecule is represented by Formula (IIb), Formula (IId), Formula (IIe), or Formula (IIf);

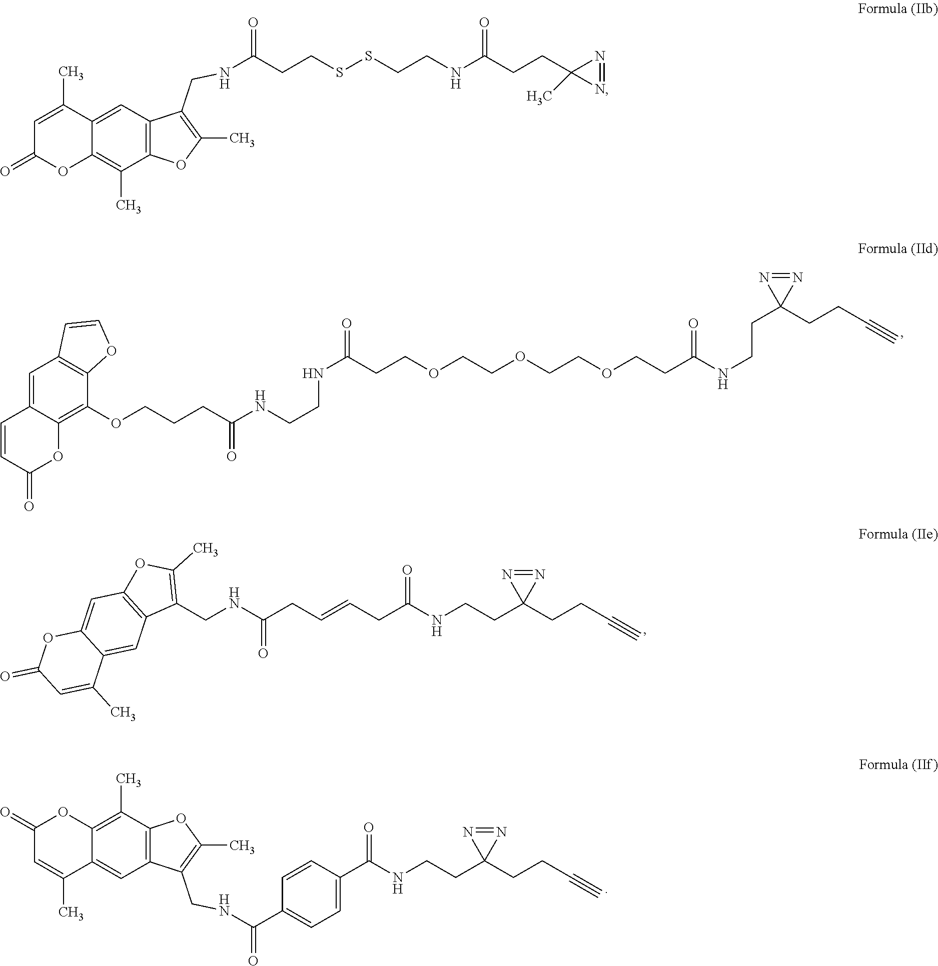

Embodiment 8A. The compound of embodiment 4A, wherein: A is selected from the group consisting of:

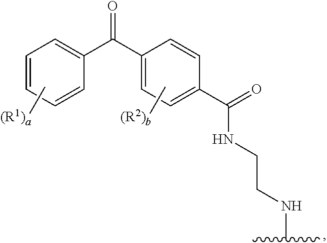

wherein:
R¹ is independently H, halo, OH, optionally substituted alkoxy, or optionally substituted alkyl;
R² is independently H, halo, OH, optionally substituted alkoxy, or optionally substituted alkyl,
a is 0, 1, 2, 3, 4, or 5; and
b is 0, 1, 2, 3, or 4;

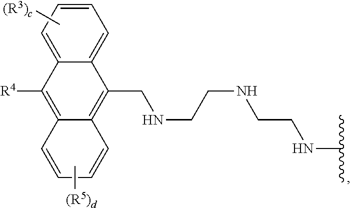

wherein:
R³ is independently H, halo, OH, optionally substituted alkoxy, or optionally substituted alkyl;
R⁴ is H, halo, OH, optionally substituted alkoxy, or optionally substituted alkyl;
R⁵ is independently H, halo, OH, optionally substituted alkoxy, or optionally substituted alkyl;
c is 0, 1, 2, 3, or 4; and
d is 0, 1, 2, 3, or 4;

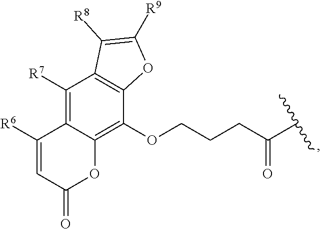

wherein:
R⁶ is H, halo, OH, optionally substituted alkoxy, or optionally substituted alkyl;
R⁷ is H, halo, OH, optionally substituted alkoxy, or optionally substituted alkyl;
R⁸ is H, halo, OH, optionally substituted alkoxy, or optionally substituted alkyl; and
R⁹ is H, halo, OH, optionally substituted alkoxy, or optionally substituted alkyl;

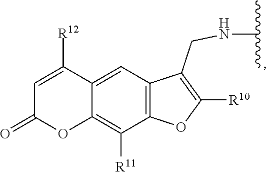

wherein:
R¹⁰ is H, halo, OH, optionally substituted alkoxy, or optionally substituted alkyl;
R¹¹ is H, halo, OH, optionally substituted alkoxy, or optionally substituted alkyl; and
R¹² is H, halo, OH, optionally substituted alkoxy, or optionally substituted alkyl;

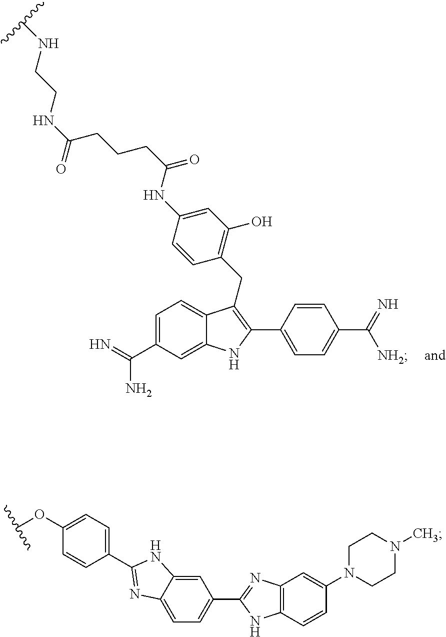 [SMILES: *NCCNC(=O)CCCC(=O)Nc1ccc(Cc2c(-c3ccc(C(=N)N)cc3)[nH]c3cc(C(=N)N)ccc23)c(O)c1 ; *Oc1ccc(-c2nc3ccc(-c4nc5cc(N6CCN(C)CC6)ccc5[nH]4)cc3[nH]2)cc1]

L1 is absent or L is selected from the group consisting of:

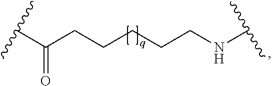

wherein:
q is 0, 1, 2, 3, or 4:

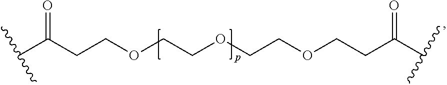

wherein:
p is 0, 1, 2, 3, or 4;

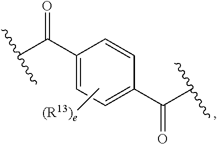

wherein:
R¹³ is independently H, halo, OH, optionally substituted alkoxy, or optionally substituted alkyl; and e is 0, 1, 2, 3 or 4;

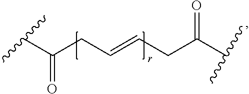

wherein:
r is 0, 1, 2, 3, or 4;

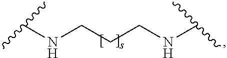

wherein:
s is 0, 1, 2, 3, or 4;

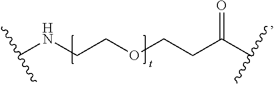

wherein:
t is 0, 1, 2, 3 or 4;

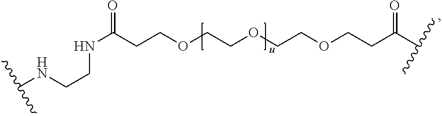

u is 0, 1, 2, 3, or 4;

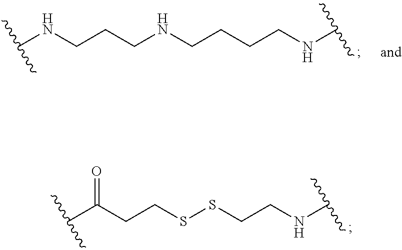 [SMILES: *NCCCCNCCCN* ; *NCCSSCCC(*)=O]

B is selected from the group consisting of:

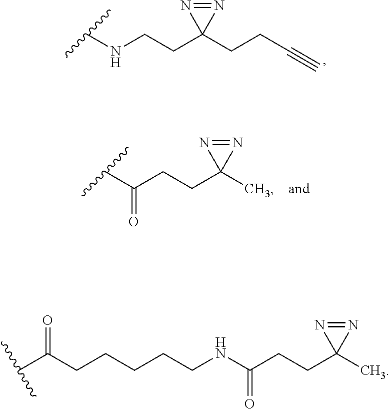 [SMILES: *NCCC1(CCC#C)N=N1 ; *C(=O)CCC1(C)N=N1 ; *C(=O)CCCCCNC(=O)CCC1(C)N=N1]

Embodiment 9A. The compound of embodiment 4A, wherein:
A is selected from the group consisting of:

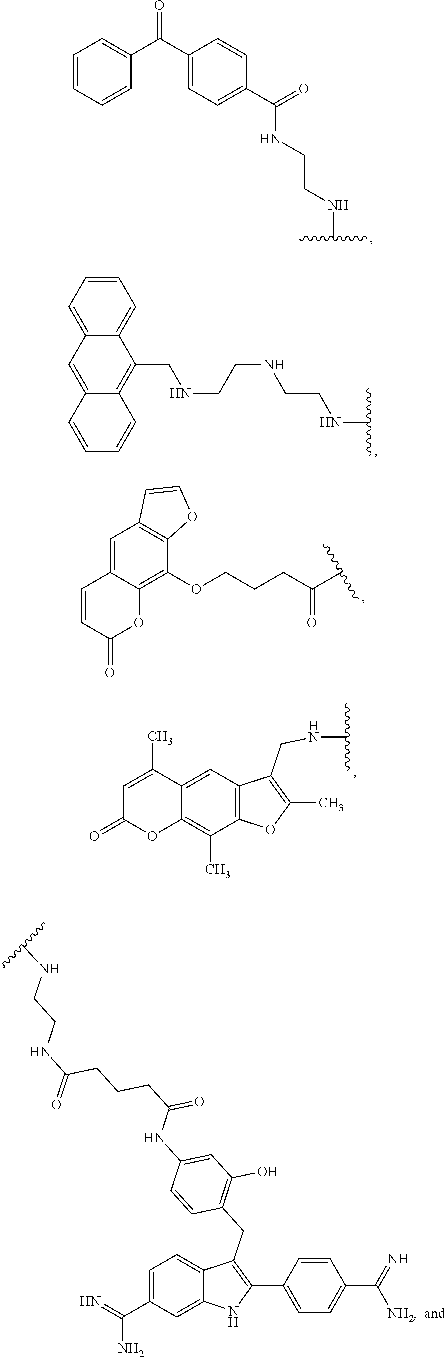 [SMILES: *NCCNC(=O)c1ccc(C(=O)c2ccccc2)cc1 ; *NCCNCCNCc1c2ccccc2cc2ccccc12 ; *C(=O)CCCOc1c2occc2cc2ccc(=O)oc12 ; *NCc1c(C)oc2c(C)c3oc(=O)cc(C)c3cc12 ; *NCCNC(=O)CCCC(=O)Nc1ccc(Cc2c(-c3ccc(C(=N)N)cc3)[nH]c3cc(C(=N)N)ccc23)c(O)c1]

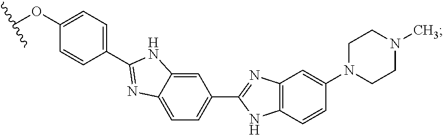 [SMILES: *Oc1ccc(-c2nc3ccc(-c4nc5cc(N6CCN(C)CC6)ccc5[nH]4)cc3[nH]2)cc1]

L1 is absent or L1 is selected from the group consisting of

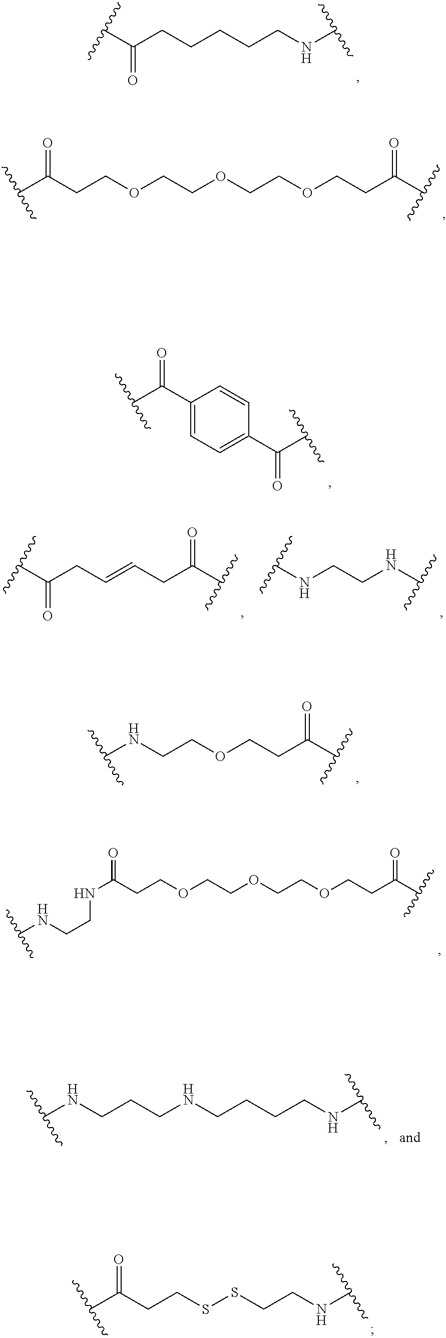

and
B is selected from the group consisting of:

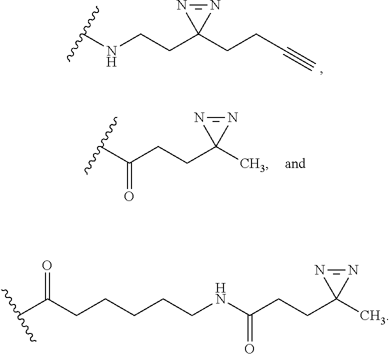 [SMILES: *NCCC1(CCC#C)N=N1 ; *C(=O)CCC1(C)N=N1 ; *C(=O)CCCCCNC(=O)CCC1(C)N=N1]

Embodiment 10A. The compound of embodiment 1A or embodiment 4A, wherein the compound is:

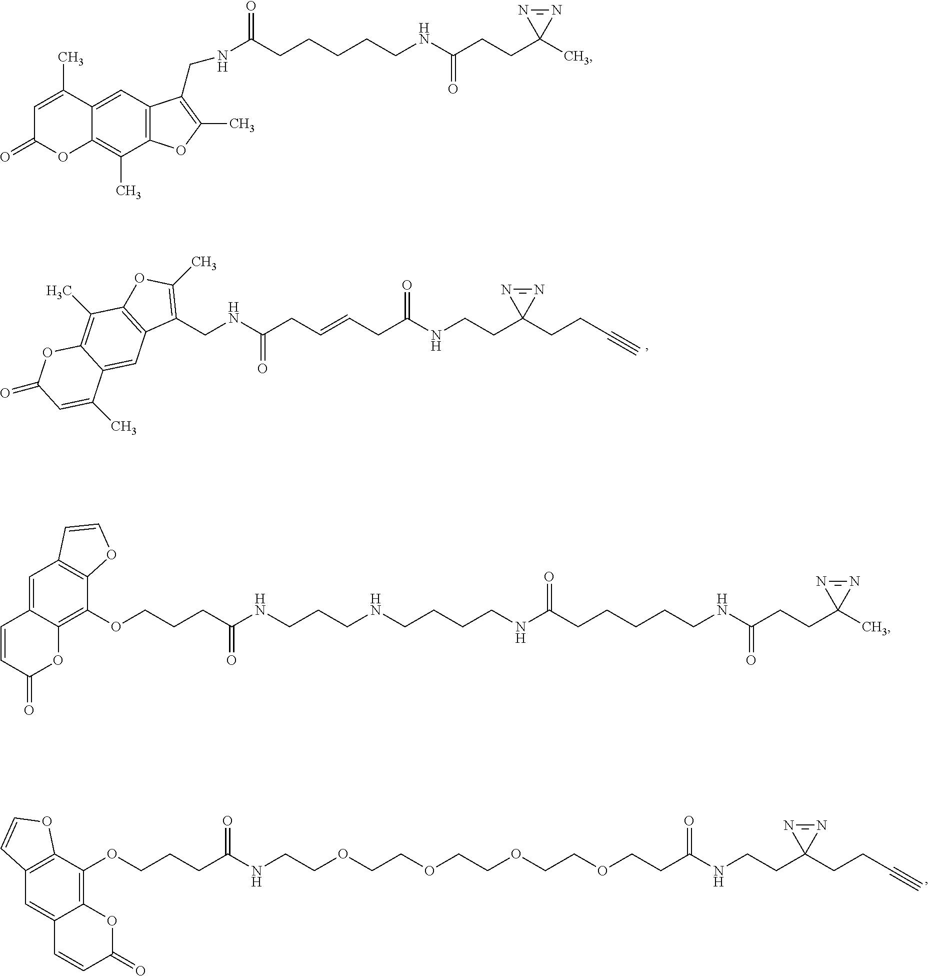 [SMILES: Cc1oc2c(C)c3oc(=O)cc(C)c3cc2c1CNC(=O)CCCCCNC(=O)CCC1(C)N=N1 ; C#CCCC1(CCNC(=O)C/C=C/CC(=O)NCc2c(C)oc3c(C)c4oc(=O)cc(C)c4cc23)N=N1 ; CC1(CCC(=O)NCCCCCC(=O)NCCCCNCCCNC(=O)CCCOc2c3occc3cc3ccc(=O)oc23)N=N1 ; C#CCCC1(CCNC(=O)CCOCCOCCOCCOCCNC(=O)CCCOc2c3occc3cc3ccc(=O)oc23)N=N1]

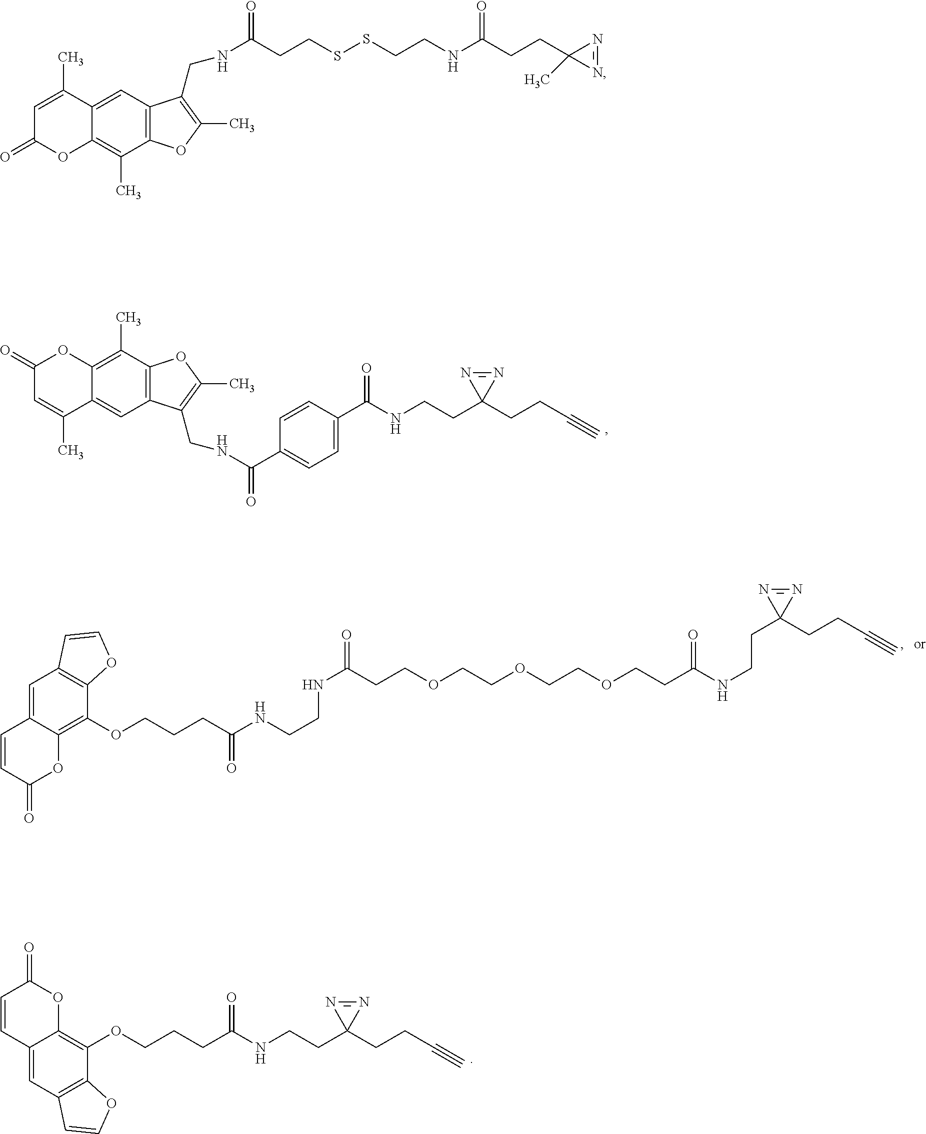 [SMILES: Cc1oc2c(C)c3oc(=O)cc(C)c3cc2c1CNC(=O)CCSSCCNC(=O)CCC1(C)N=N1 ; C#CCCC1(CCNC(=O)c2ccc(C(=O)NCc3c(C)oc4c(C)c5oc(=O)cc(C)c5cc34)cc2)N=N1 ; C#CCCC1(CCNC(=O)CCOCCOCCOCCC(=O)NCCNC(=O)CCCOc2c3occc3cc3ccc(=O)oc23)N=N1 ; C#CCCC1(CCNC(=O)CCCOc2c3occc3cc3ccc(=O)oc23)N=N1]

Embodiment 11A. The compound of embodiment 5A, wherein L1 comprises 2 to 20 carbons or 20-100 carbons in length.

Embodiment 12A. The compound of any one of embodiments 1A-3A, wherein n=1, m is an integer being 2 or greater, and C represents a core moiety having at least three functional groups each separately for attachment to L1 and attachment to the at least two arms each represented by (L2-B), so that the compound is represented by Formula (III):

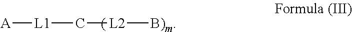

Embodiment 13A. The compound of embodiment 12A, wherein B comprises diazirine or an azide diazirine in one of the at least two arms, and B represents a detectable functional group in another one of the at least two arms, said detectable function group comprising a fluorophore, a biotin, a chromophore, a chromogen, a quantum dot, a fluorescent microsphere, or a nanoparticle.

Embodiment 14A. The compound of embodiment 12A or embodiment 13A, wherein L1, L2, or both independently comprise one or more of (i) a cleavable bond, (ii) an oligomer or polymer having a repeating unit of —OCH₂CH₂—, and (iii) an unsaturated moiety.

Embodiment 15A. The compound of any one of embodiments 9A-14A, wherein C represents a dendritic core moiety comprising at least three surface functional groups each separately for attachment to L1 and attachment to the at least two arms each represented by L2-B.

Embodiment 16A. The compound of any one of embodiments 9A-15A, wherein L1, L2, or both independently comprise a triazole in bonding with A.

Embodiment 17A. A method of crosslinking a nucleic acid with a protein in proximity in a system, comprising: incubating the compound of any one of embodiments 1A-16A with the system, and irradiating the system with an ultraviolet light.

Embodiment 18A. The method of embodiment 17A, wherein the system is a live cell.

Embodiment 19A. The method of embodiment 17A or embodiment 18A, wherein the ultraviolet light is between 300 nm and 370 nm in wavelength.

Embodiment 20A. The method of any one of embodiments 17A-19A, further comprising performing one or more of immuno precipitation, chromatic precipitation, 3D chromatin conformation capture, mass spectrometry, and electrophoresis, with the system.

Embodiment 21A. The method of any one of embodiments 17A-20A, wherein element L1, L2, or both of the compound is independently cleavable, and the method further comprises adding a cleaving agent to the system to cleave the elements L1, L2, or both; or wherein element A of the compound is derived from psoralen, and the method further comprises applying an ultraviolet light of about 230 nm in wavelength to cleave the element A; thereby generating a fingerprint of crosslinked proteins in proximity to nucleic acids in the system.

Embodiment 22A. A method for preparing the compound of any one of embodiments 12A-16A, comprising:
providing an azide derivative of a nucleic acid-binding, photo-reactive agent comprising psoralen, methyltrioxsalen, benzophenone, 4′,6-diamidino-2-phenylindole (DAPI), a Hoechst dye, a polyamide, or a G quartet binding molecule, kethoxal, or a derivative thereof;
providing an azide derivative of a photo-reactive agent that comprises a diazirine moiety so as to obtain an azide-diazirine bifunctional, photo-reactive agent, and said photo-reactive agent optionally further comprising an alkyne group, or providing an aryl azide, said aryl azide optionally selected from phenyl azide, orthro-hydroxyphenyl azide, meta-hydroxyphenyl azide, tetrafluorophenyl azide, ortho-nitropenyl azide, meta-nitropenyl azide or azdo-methylcoumarin;
optionally providing an azide derivative of a detectable agent comprising a fluorophore, a biotin, a chromophore, a chromogen, a quantum dot, a fluorescent microsphere, or a nanoparticle;
providing a multi-arm agent having at least three functional groups each independently comprising an alkyne; and
combining each azide derivatives and, if provided, the aryl azide, together with the multi-arm agent in one reaction vessel to prepare the compound.

Embodiment 23A. The method of embodiment 22A, wherein the multi-arm agent has at least three functional groups each independently comprising a cyclooctyne group.

Embodiment 24A. The method of embodiment 22A or embodiment 23A, wherein the nucleic acid-binding, photo-reactive agent comprises a first primary amine functional group, and providing the azide derivative of the nucleic acid-binding, photo-reactive agent comprises converting the first primary amine functional group to a first azide-containing moiety, optionally via reacting the nucleic acid-binding, photo-reactive agent with imidazole-1-sulfonyl azide; and/or wherein the photo-reactive agent that comprises a diazirine moiety further comprises a second primary amine functional group or is modified with the second primary amino functional group, and providing the azide derivative of said photo-reactive agent comprises converting the second primary amine functional group to a second azide-containing moiety, optionally via reacting said photo-reactive agent with imidazole-1-sulfonyl azide.

Embodiment 25A. A method of crosslinking a nucleic acid with a protein in a system, comprising: providing a compound of any one of embodiments 1A-16A;
providing a system, wherein the system comprises a nucleic acid and a protein;
contacting the compound with the system; and
irradiating the system and the compound with an ultraviolet light under conditions effective to crosslink the nucleic acid with the protein.

Embodiment 26A. The method of embodiment 25A, wherein the system is a live cell.

Embodiment 27A. The method of embodiment 25A or embodiment 26A, wherein the ultraviolet light is between 300 nm and 370 nm in wavelength.

Embodiment 28A. The method of any one of embodiments 25A-27A, further comprising performing one or more of immuno precipitation, chromatic precipitation, 3D chromatin conformation capture, mass spectrometry, and electrophoresis, with the system.

Embodiment 29A. The method of any one of embodiments 25A-28A, wherein element L1, L2, or both of the compound is independently cleavable, and the method further comprises adding a cleaving agent to the system to cleave the elements L1, L2, or both; or wherein element A of the compound is derived from psoralen, and the method further comprises applying an ultraviolet light of about 230 nm in wavelength to cleave the element A; thereby generating a fingerprint of crosslinked proteins in proximity to nucleic acids in the system.

Additional embodiments include those listed below.

In some embodiments, the present invention provides a compound:

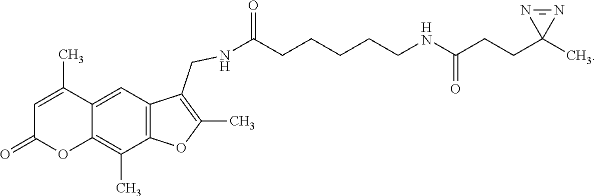 [SMILES: Cc1oc2c(C)c3oc(=O)cc(C)c3cc2c1CNC(=O)CCCCCNC(=O)CCC1(C)N=N1]

In some embodiments, the present invention provides a compound:

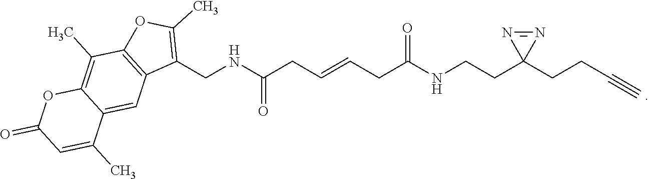 [SMILES: C#CCCC1(CCNC(=O)C/C=C/CC(=O)NCc2c(C)oc3c(C)c4oc(=O)cc(C)c4cc23)N=N1]

In some embodiments, the present invention provides a compound:

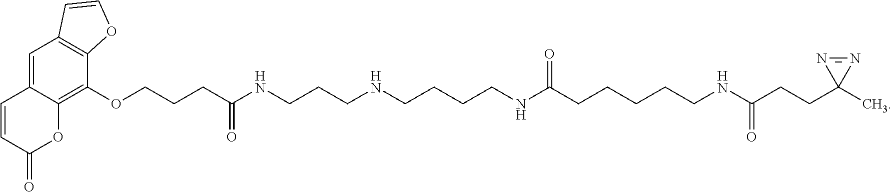 [SMILES: CC1(CCC(=O)NCCCCCC(=O)NCCCCNCCCNC(=O)CCCOc2c3occc3cc3ccc(=O)oc23)N=N1]

In some embodiments, the present invention provides a compound:

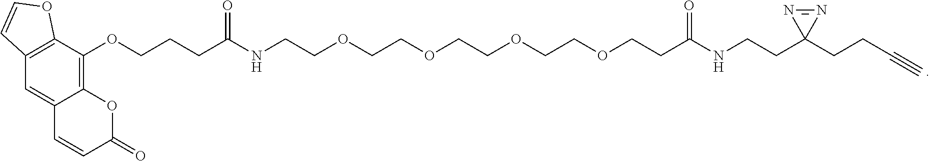 [SMILES: C#CCCC1(CCNC(=O)CCOCCOCCOCCOCCNC(=O)CCCOc2c3occc3cc3ccc(=O)oc23)N=N1]

In some embodiments, the present invention provides a compound:

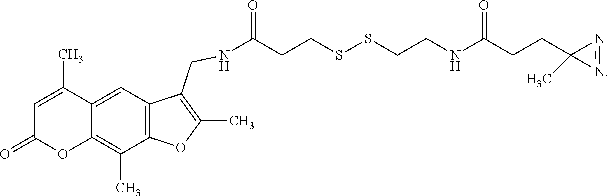 [SMILES: Cc1oc2c(C)c3oc(=O)cc(C)c3cc2c1CNC(=O)CCSSCCNC(=O)CCC1(C)N=N1]

In some embodiments, the present invention provides a compound:

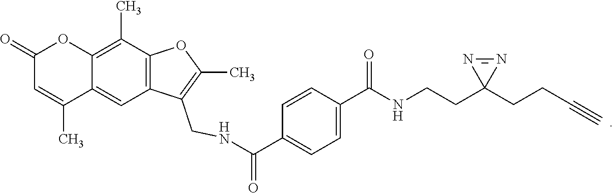 [SMILES: C#CCCC1(CCNC(=O)c2ccc(C(=O)NCc3c(C)oc4c(C)c5oc(=O)cc(C)c5cc34)cc2)N=N1]

In some embodiments, the present invention provides a compound:

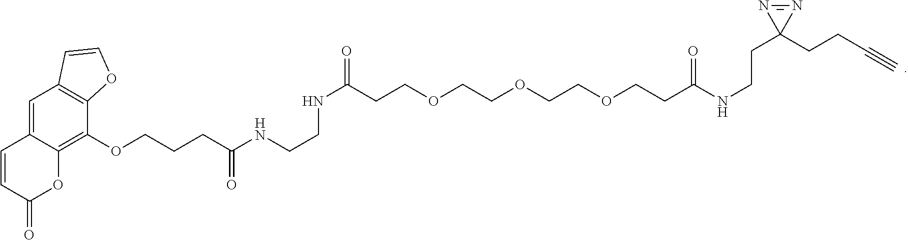 [SMILES: C#CCCC1(CCNC(=O)CCOCCOCCOCCC(=O)NCCNC(=O)CCCOc2c3occc3cc3ccc(=O)oc23)N=N1]

In some embodiments, the present invention provides a compound:

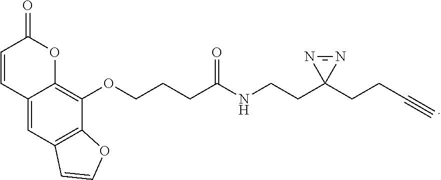 [SMILES: C#CCCC1(CCNC(=O)CCCOc2c3occc3cc3ccc(=O)oc23)N=N1]

Additional embodiments include those listed below.

In some embodiments, a compound of the present invention is a compound of Formula (I), a compound of Formula (II), a compound of Formula (III), a compound of Formula (IIa), a compound of Formula (IIb), a compound of Formula (IIc), a compound of Formula (IId), or a compound of Formula (IIe), or any combination thereof. In some embodiments, a compound of the present invention is a compound of Formula (II).

In various embodiments, the present invention provides a compound of Formula (II): A-L1-B. In some embodiments, L1 is absent. In some embodiments, L1 is present. In some embodiments, L1 is absent or the first linker.

Additional embodiments include those listed below.

In some embodiments, a compound of the present invention is selected from the group consisting of:

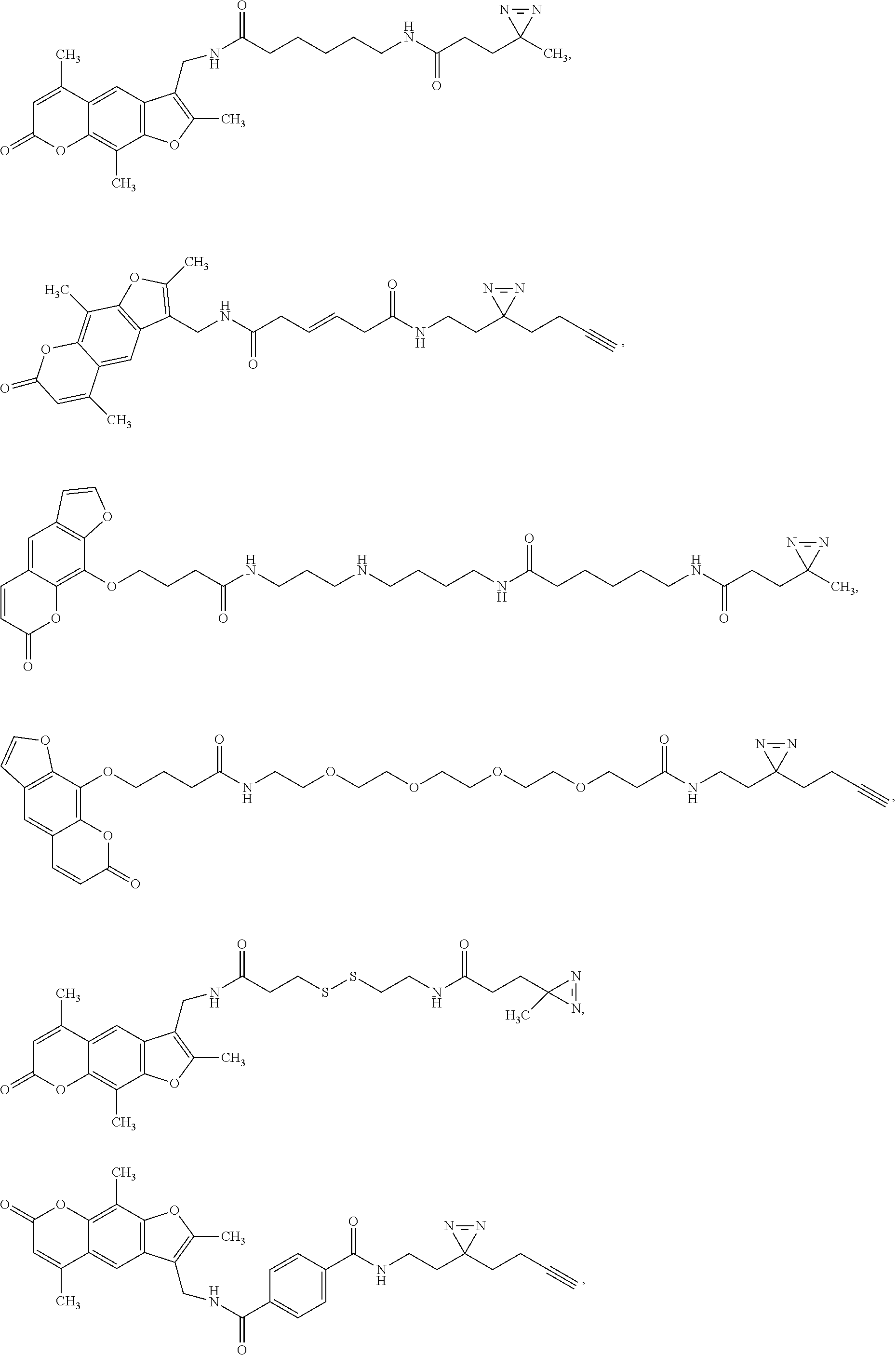 [SMILES: Cc1oc2c(C)c3oc(=O)cc(C)c3cc2c1CNC(=O)CCCCCNC(=O)CCC1(C)N=N1 ; C#CCCC1(CCNC(=O)C/C=C/CC(=O)NCc2c(C)oc3c(C)c4oc(=O)cc(C)c4cc23)N=N1 ; CC1(CCC(=O)NCCCCCC(=O)NCCCCNCCCNC(=O)CCCOc2c3occc3cc3ccc(=O)oc23)N=N1 ; C#CCCC1(CCNC(=O)CCOCCOCCOCCOCCNC(=O)CCCOc2c3occc3cc3ccc(=O)oc23)N=N1 ; Cc1oc2c(C)c3oc(=O)cc(C)c3cc2c1CNC(=O)CCSSCCNC(=O)CCC1(C)N=N1 ; C#CCCC1(CCNC(=O)c2ccc(C(=O)NCc3c(C)oc4c(C)c5oc(=O)cc(C)c5cc34)cc2)N=N1]

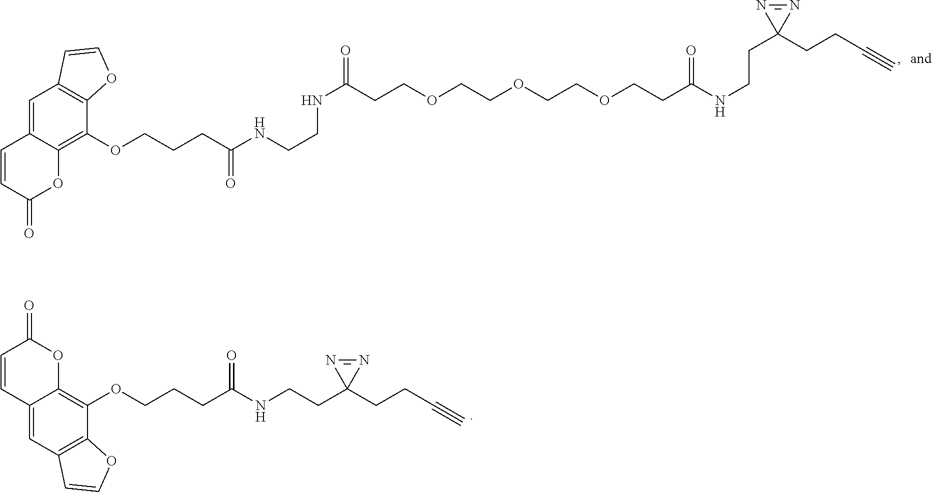 [SMILES: C#CCCC1(CCNC(=O)CCOCCOCCOCCC(=O)NCCNC(=O)CCCOc2c3occc3cc3ccc(=O)oc23)N=N1 ; C#CCCC1(CCNC(=O)CCCOc2c3occc3cc3ccc(=O)oc23)N=N1]

Additional embodiments include those listed below.

In various embodiments, the present invention provides a compound of Formula (II): A-L1-B. In some embodiments, L1 is absent. In some embodiments, L1 is present. In some embodiments, L1 is absent or the first linker. In some embodiments, a compound of Formula (II) is selected from the group consisting of:

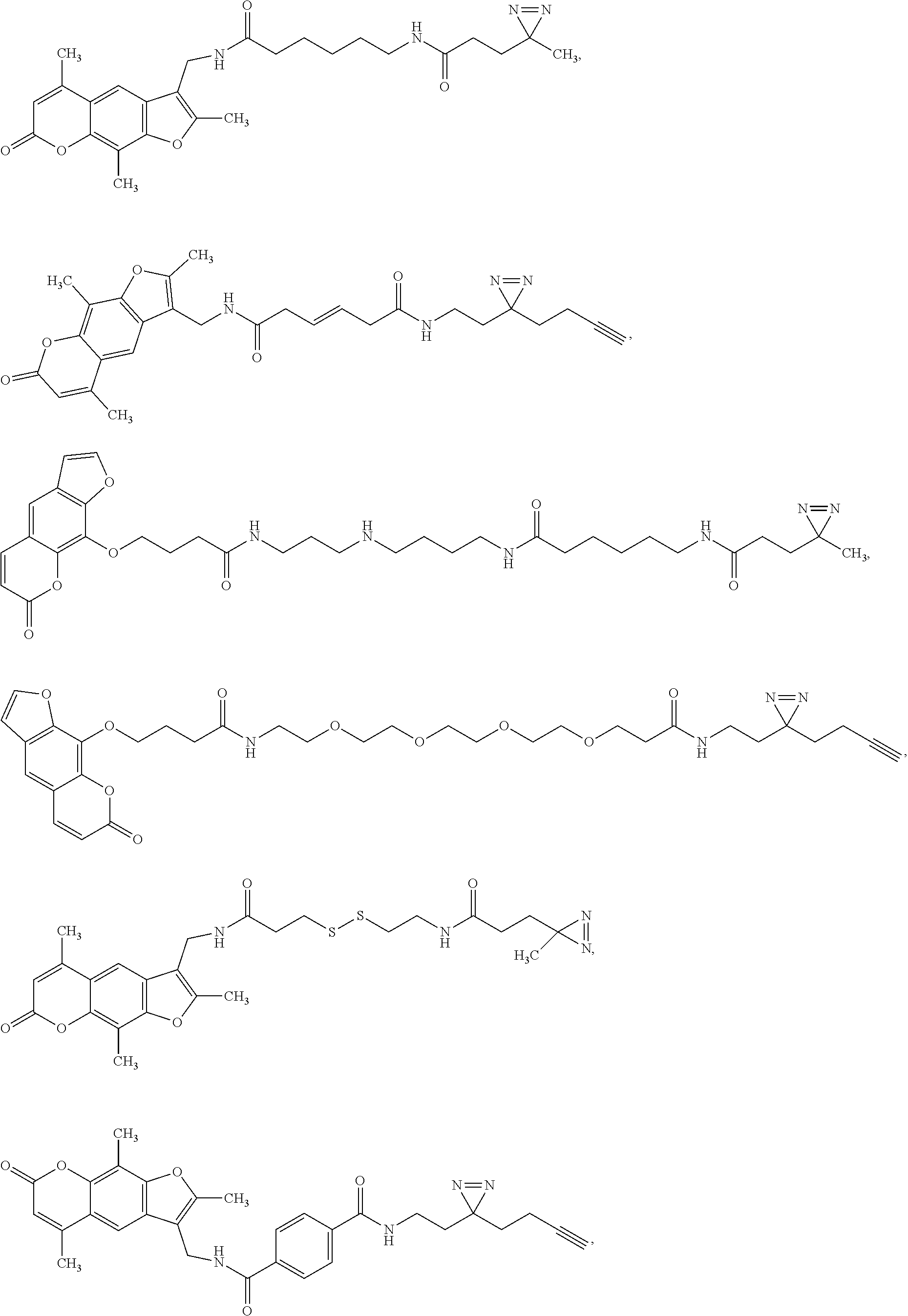

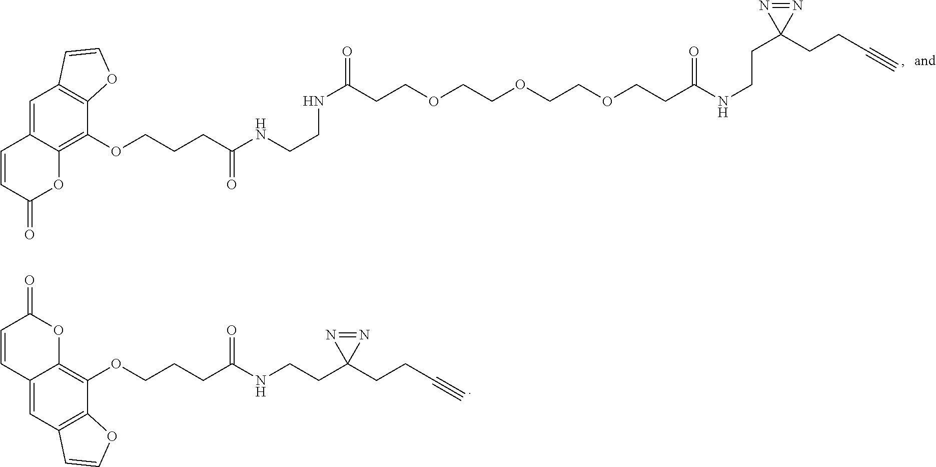

Additional embodiments include those listed below.

In some embodiments, a compound of Formula (I) is selected from the group consisting of:

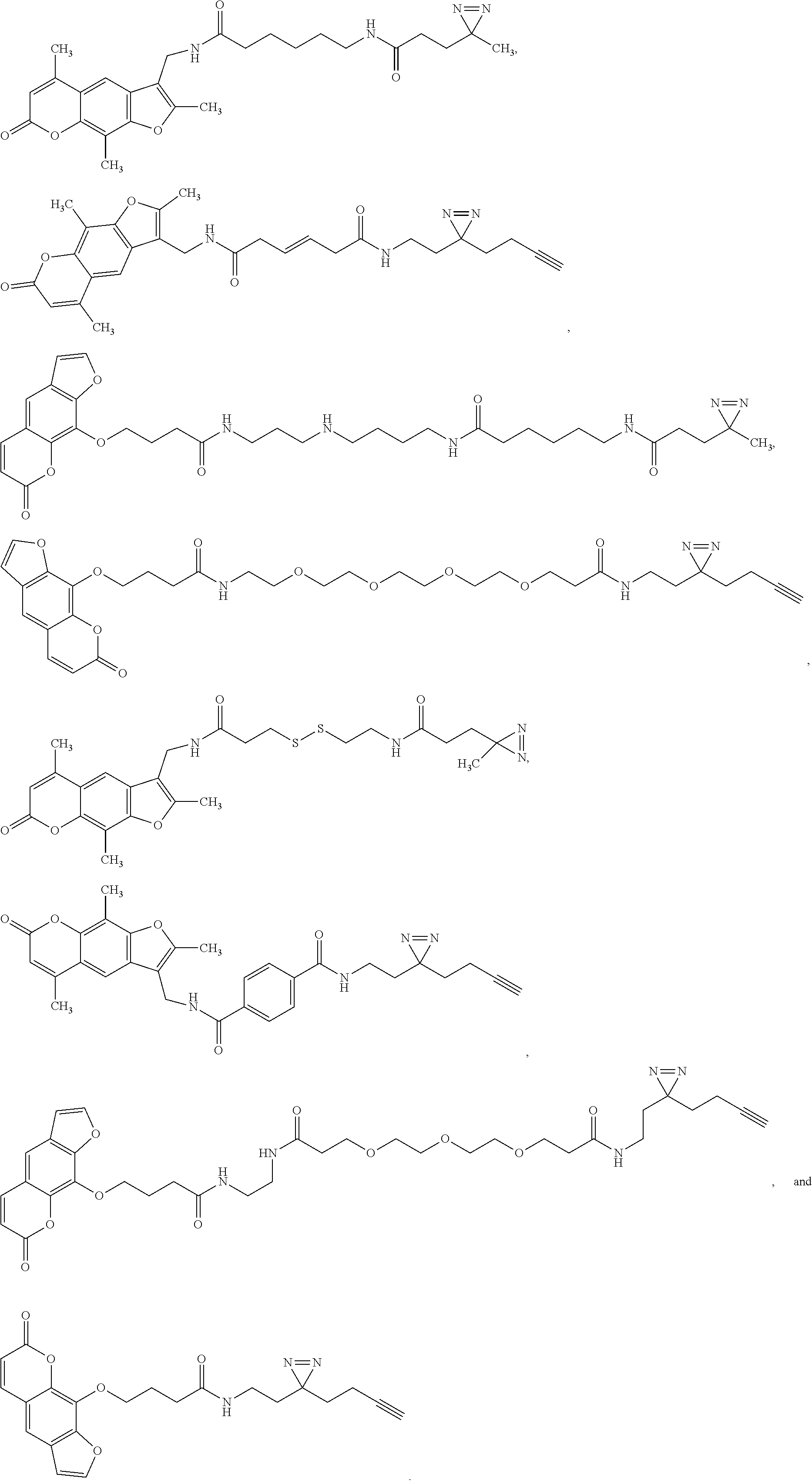

Additional embodiments include those listed below.

In some embodiments, A is selected from the group consisting of:

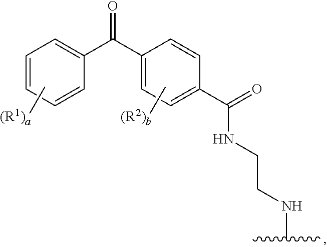

wherein:
R¹ is independently H, halo, OH, optionally substituted alkoxy, or optionally substituted alkyl;
R² is independently H, halo, OH, optionally substituted alkoxy, or optionally substituted alkyl;
a is 0, 1, 2, 3, 4, or 5; and
b is 0, 1, 2, 3, or 4;

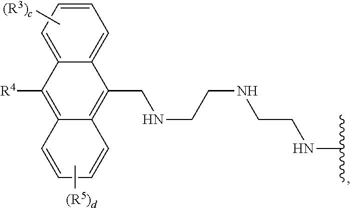

wherein:
R³ is independently H, halo, OH, optionally substituted alkoxy, or optionally substituted alkyl,
R⁴ is H, halo, OH, optionally substituted alkoxy, or optionally substituted alkyl;
R⁵ is independently H, halo, OH, optionally substituted alkoxy, or optionally substituted alkyl;
c is 0, 1, 2, 3, or 4; and
d is 0, 1, 2, 3, or 4;

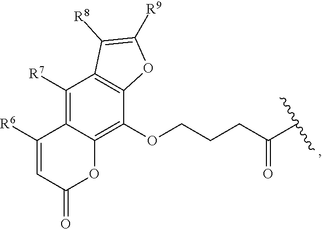

wherein:
R⁶ is H, halo, OH, optionally substituted alkoxy, or optionally substituted alkyl,
R⁷ is H, halo, OH, optionally substituted alkoxy, or optionally substituted alkyl;
R⁸ is H, halo, OH, optionally substituted alkoxy, or optionally substituted alkyl; and
R⁹ is H, halo, OH, optionally substituted alkoxy, or optionally substituted alkyl;

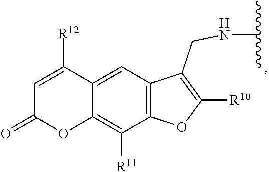

wherein:
R¹⁰ is H, halo, OH, optionally substituted alkoxy, or optionally substituted alkyl;
R¹¹ is H, halo, OH, optionally substituted alkoxy, or optionally substituted alkyl; and
R¹² is H, halo, OH, optionally substituted alkoxy, or optionally substituted alkyl;

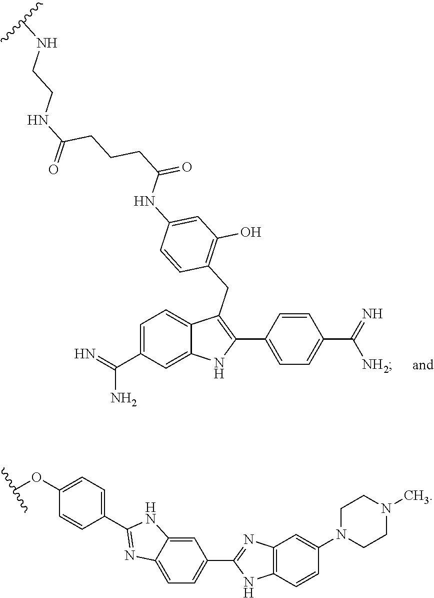 [SMILES: *NCCNC(=O)CCCC(=O)Nc1ccc(Cc2c(-c3ccc(C(=N)N)cc3)[nH]c3cc(C(=N)N)ccc23)c(O)c1 ; *Oc1ccc(-c2nc3ccc(-c4nc5cc(N6CCN(C)CC6)ccc5[nH]4)cc3[nH]2)cc1]

Additional embodiments include those listed below.

In some embodiments, A is:

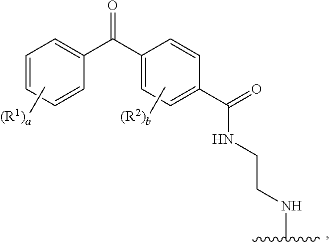

wherein:
R¹ is independently H, halo, OH, optionally substituted alkoxy, or optionally substituted alkyl; R² is independently H, halo, OH, optionally substituted alkoxy, or optionally substituted alkyl;
a is 0, 1, 2, 3, 4, or 5; and
b is 0, 1, 2, 3, or 4.

In some embodiments, A is:

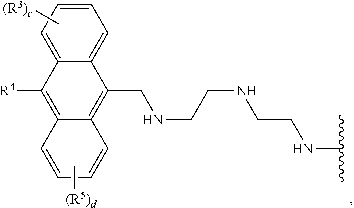

wherein:
R³ is independently H, halo, OH, optionally substituted alkoxy, or optionally substituted alkyl,
R⁴ is H, halo, OH, optionally substituted alkoxy, or optionally substituted alkyl;
R⁵ is independently H, halo, OH, optionally substituted alkoxy, or optionally substituted alkyl;
c is 0, 1, 2, 3, or 4; and
d is 0, 1, 2, 3, or 4.

In some embodiments, A is:

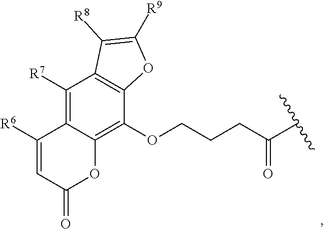

wherein:
R⁶ is H, halo, OH, optionally substituted alkoxy, or optionally substituted alkyl;
R⁷ is H, halo, OH, optionally substituted alkoxy, or optionally substituted alkyl;
R⁸ is H, halo, OH, optionally substituted alkoxy, or optionally substituted alkyl; and
R⁹ is H, halo, OH, optionally substituted alkoxy, or optionally substituted alkyl

In some embodiments, A is:

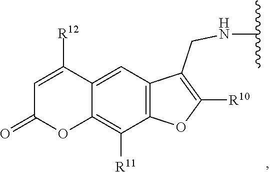

wherein:
R¹⁰ is H, halo, OH, optionally substituted alkoxy, or optionally substituted alkyl;
R¹¹ is H, halo, OH, optionally substituted alkoxy, or optionally substituted alkyl; and
R¹² is H, halo, OH, optionally substituted alkoxy, or optionally substituted alkyl.

In some embodiments, A is:

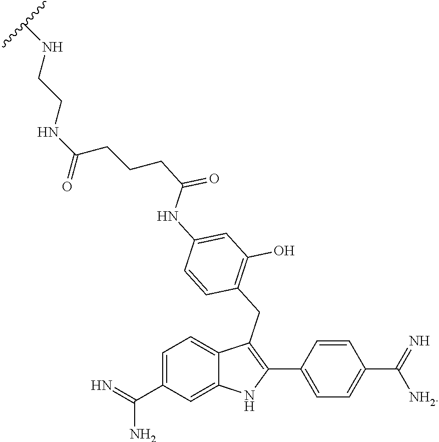 [SMILES: *NCCNC(=O)CCCC(=O)Nc1ccc(Cc2c(-c3ccc(C(=N)N)cc3)[nH]c3cc(C(=N)N)ccc23)c(O)c1]

In some embodiments, A is:

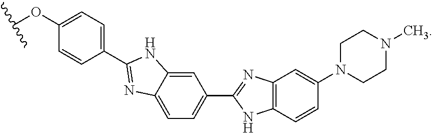 [SMILES: *Oc1ccc(-c2nc3ccc(-c4nc5cc(N6CCN(C)CC6)ccc5[nH]4)cc3[nH]2)cc1]

Additional embodiments include those listed below.

In some embodiments, A is selected from the group consisting of:

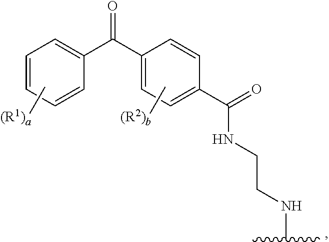

wherein:
R¹ is independently H, halo, OH, OCH₃, or CH₃;
R² is independently H, halo, OH, OCH₃, or CH₃;
a is 0, 1, 2, 3, 4, or 5; and
b is 0, 1, 2, 3, or 4;

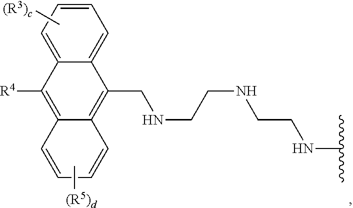

wherein:
R³ is independently H, halo, OH, OCH₃, or CH₃;
R⁴ is H, halo, OH, OCH₃, or CH₃;
R⁵ is independently H, halo, OH, OCH₃, or CH₃;
c is 0, 1, 2, 3, or 4; and
d is 0, 1, 2, 3, or 4;

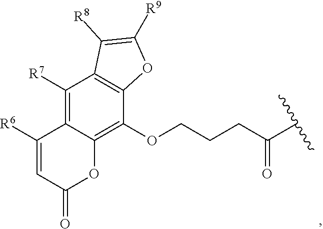

wherein:
R⁶ is H, halo, OH, OCH₃, or CH₃;
R⁷ is H, halo, OH, OCH₃, or CH₃;
R⁸ is H, halo, OH, OCH₃, or CH₃; and
R⁹ is H, halo, OH, OCH₃, or CH₃;

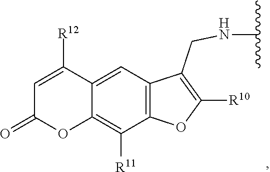

wherein:
R¹⁰ is H, halo, OH, OCH₃, or CH₃;
R¹¹ is H, halo, OH, OCH₃, or CH₃; and
R¹² is H, halo, OH, OCH₃, or CH₃;

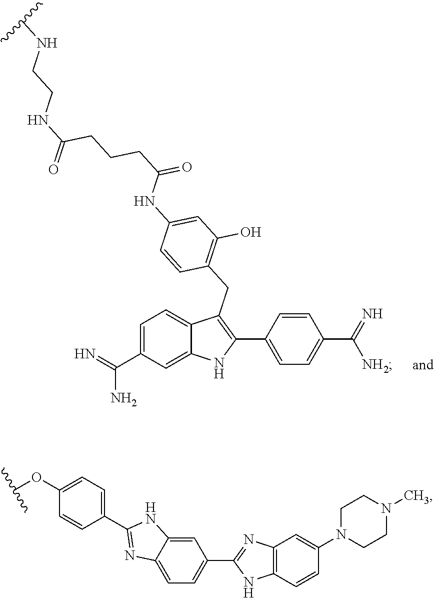 [SMILES: *NCCNC(=O)CCCC(=O)Nc1ccc(Cc2c(-c3ccc(C(=N)N)cc3)[nH]c3cc(C(=N)N)ccc23)c(O)c1 ; *Oc1ccc(-c2nc3ccc(-c4nc5cc(N6CCN(C)CC6)ccc5[nH]4)cc3[nH]2)cc1]

Additional embodiments include those listed below.

In some embodiments, A is:

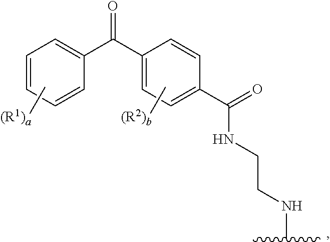

wherein:
R¹ is independently H, halo, OH, OCH₃, or CH₃;
R² is independently H, halo, OH, OCH₃, or CH₃;
a is 0, 1, 2, 3, 4, or 5; and
b is 0, 1, 2, 3, or 4.

In some embodiments, A is:

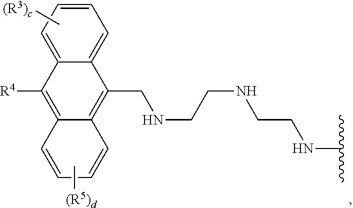

wherein:
R³ is independently H, halo, OH, OCH₃, or CH₃;
R⁴ is H, halo, OH, OCH₃, or CH₃;
R⁵ is independently H, halo, OH, OCH₃, or CH₃;
c is 0, 1, 2, 3, or 4; and
d is 0, 1, 2, 3, or 4.

In some embodiments, A is:

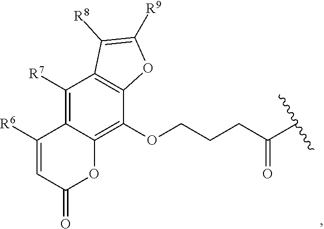

wherein:
R⁶ is H, halo, OH, OCH₃, or CH₃;
R⁷ is H, halo, OH, OCH₃, or CH₃;
R⁸ is H, halo, OH, OCH₃, or CH₃; and
R⁹ is H, halo, OH, OCH₃, or CH₃.

In some embodiments, A is:

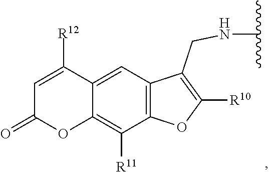

wherein:
R¹⁰ is H, halo, OH, OCH₃, or CH₃;
R¹¹ is H, halo, OH, OCH₃, or CH₃; and
R¹² is H, halo, OH, OCH₃, or CH₃.

In some embodiments, A is:

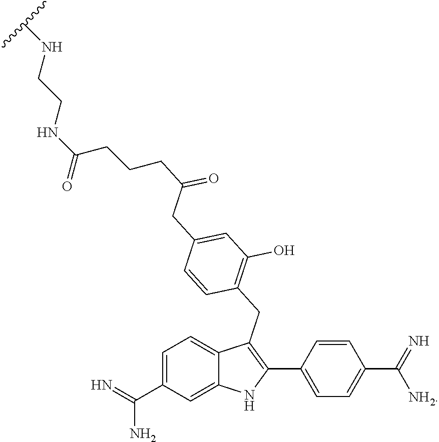 [SMILES: *NCCNC(=O)CCCC(=O)Cc1ccc(Cc2c(-c3ccc(C(=N)N)cc3)[nH]c3cc(C(=N)N)ccc23)c(O)c1]

In some embodiments, A is:

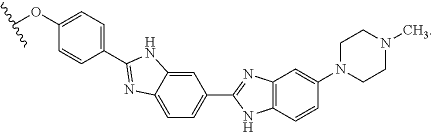 [SMILES: *Oc1ccc(-c2nc3ccc(-c4nc5cc(N6CCN(C)CC6)ccc5[nH]4)cc3[nH]2)cc1]

Additional embodiments include those listed below.

In some embodiments, A is selected from the group consisting of:

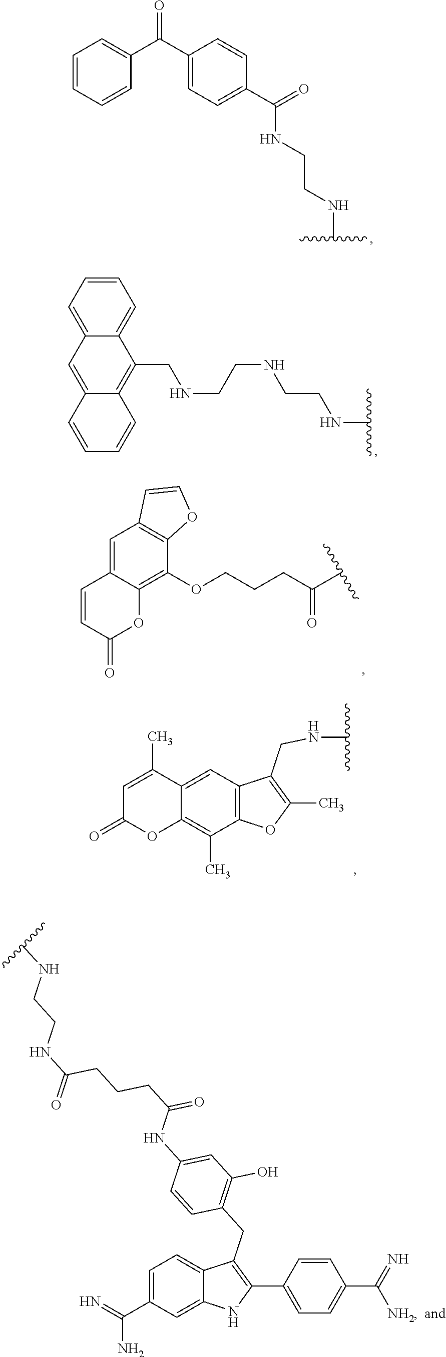 [SMILES: *NCCNC(=O)c1ccc(C(=O)c2ccccc2)cc1 ; *NCCNCCNCc1c2ccccc2cc2ccccc12 ; *C(=O)CCCOc1c2occc2cc2ccc(=O)oc12 ; *NCc1c(C)oc2c(C)c3oc(=O)cc(C)c3cc12 ; *NCCNC(=O)CCCC(=O)Nc1ccc(Cc2c(-c3ccc(C(=N)N)cc3)[nH]c3cc(C(=N)N)ccc23)c(O)c1]

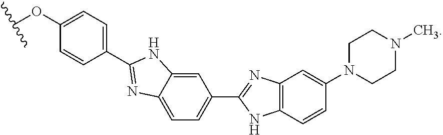 [SMILES: *Oc1ccc(-c2nc3ccc(-c4nc5cc(N6CCN(C)CC6)ccc5[nH]4)cc3[nH]2)cc1]

In some embodiments, A is selected from the group consisting of:

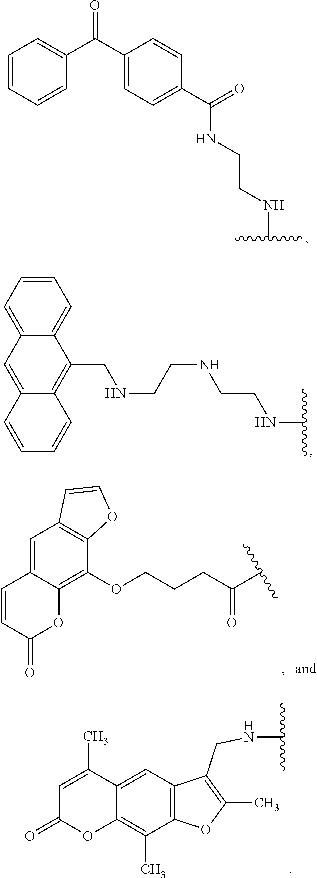

In some embodiments, A is:

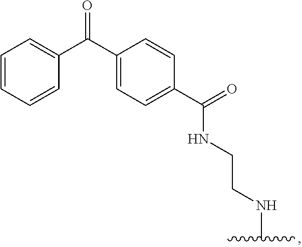 [SMILES: *NCCNC(=O)c1ccc(C(=O)c2ccccc2)cc1]

In some embodiments, A is:

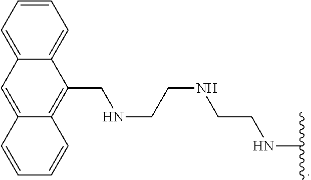 [SMILES: *NCCNCCNCc1c2ccccc2cc2ccccc12]

In some embodiments, A is:

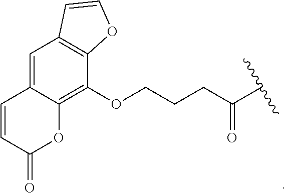 [SMILES: *C(=O)CCCOc1c2occc2cc2ccc(=O)oc12]

In some embodiments, A is:

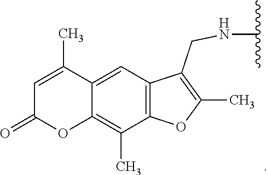 [SMILES: *NCc1c(C)oc2c(C)c3oc(=O)cc(C)c3cc12]

In some embodiments, A is:

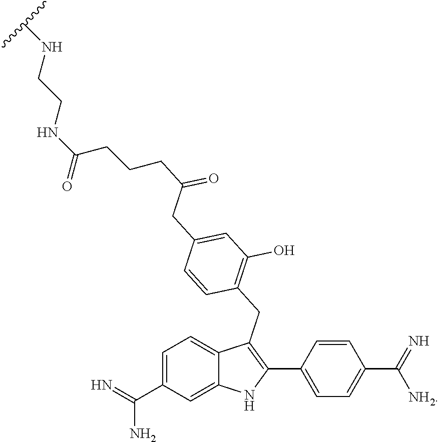 [SMILES: *NCCNC(=O)CCCC(=O)Cc1ccc(Cc2c(-c3ccc(C(=N)N)cc3)[nH]c3cc(C(=N)N)ccc23)c(O)c1]

In some embodiments, A is:

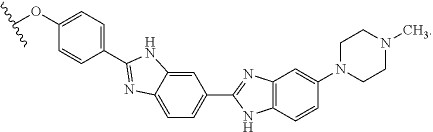 [SMILES: *Oc1ccc(-c2nc3ccc(-c4nc5cc(N6CCN(C)CC6)ccc5[nH]4)cc3[nH]2)cc1]

Additional embodiments include those listed below.

In some embodiments, L1 is selected from the group consisting of:

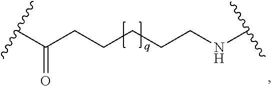

wherein:
q is 0, 1, 2, 3, or 4.

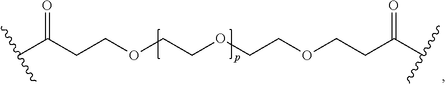

wherein:
p is 0, 1, 2, 3, or 4;

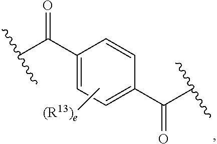

wherein:
R¹³ is independently H, halo, OH, optionally substituted alkoxy, or optionally substituted alkyl and
e is 0, 1, 2, 3 or 4:

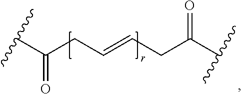

wherein:
r is 0, 1, 2, 3, or 4;

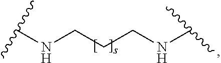

wherein:
s is 0, 1, 2, 3, or 4;

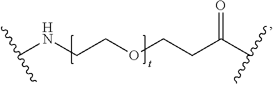

wherein:
t is 0, 1, 2, 3 or 4;

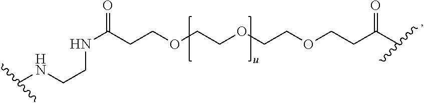

wherein:
u is 0, 1, 2, 3, or 4;

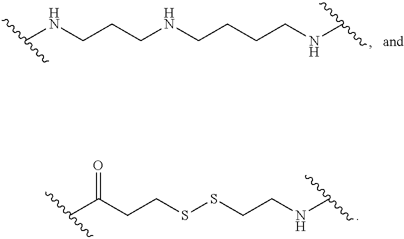 [SMILES: *NCCCCNCCCN* ; *NCCSSCCC(*)=O]

In some embodiments, L1 is:

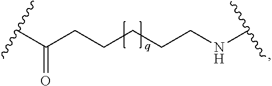

wherein:
q is 0, 1, 2, 3, or 4.

In some embodiments, L1 is:

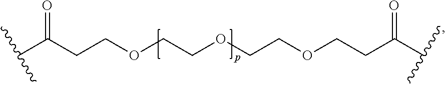

wherein:
p is 0, 1, 2, 3, or 4.

In some embodiments, L1 is:

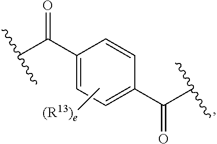

wherein:
R¹³ is independently H, halo, OH, optionally substituted alkoxy, or optionally substituted alkyl; and
e is 0, 1, 2, 3 or 4.

In some embodiments, L1 is:

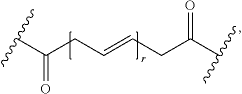

wherein:
r is 0, 1, 2, 3, or 4.

In some embodiments, L1 is:

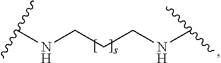

wherein:
s is 0, 1, 2, 3, or 4.

In some embodiments, L1 is:

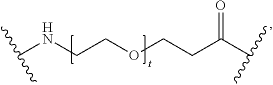

wherein:
t is 0, 1, 2, 3 or 4.

In some embodiments, L1 is:

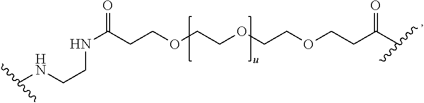

wherein:
u is 0, 1, 2, 3, or 4

In some embodiments, L1 is:

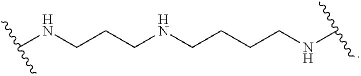 [SMILES: *NCCCCNCCCN*]

In some embodiments, L1 is:

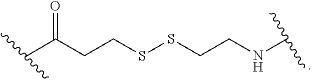 [SMILES: *NCCSSCCC(*)=O]

In some embodiments, L1 is selected from the group consisting of:

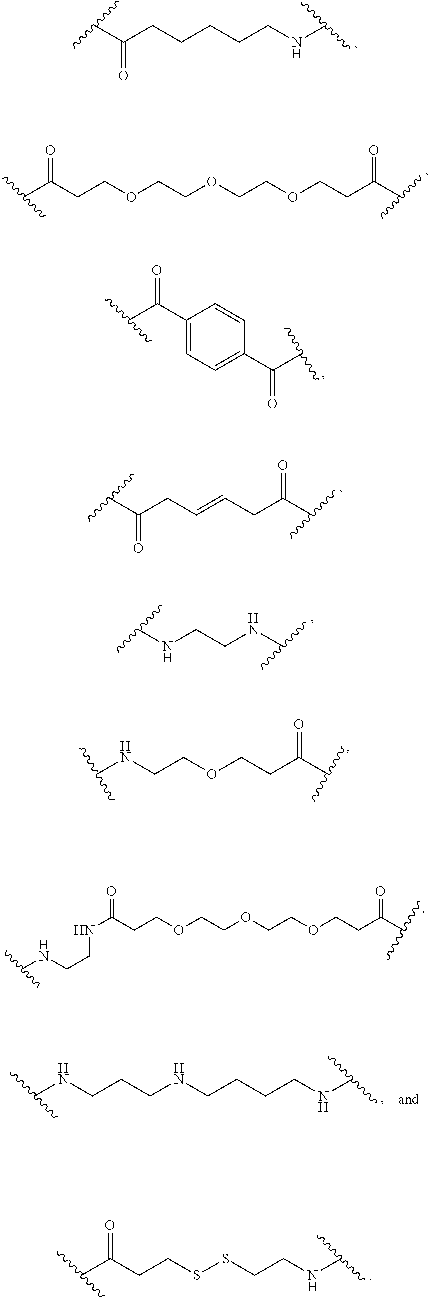

In some embodiments, L1 is:

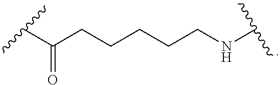 [SMILES: *NCCCCCC(*)=O]

In some embodiments, L1 is:

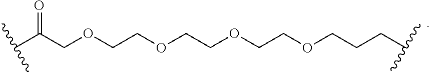 [SMILES: *CCCOCCOCCOCCOCC(*)=O]

In some embodiments, L1 is:

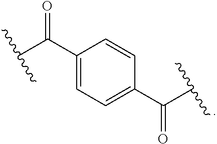 [SMILES: *C(=O)c1ccc(C(*)=O)cc1]

In some embodiments, L1 is:

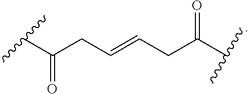 [SMILES: *C(=O)C/C=C/CC(*)=O]

In some embodiments, L1 is:

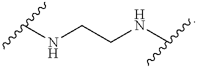 [SMILES: *NCCN*]

In some embodiments, L1 is:

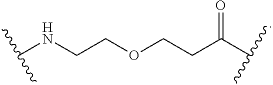 [SMILES: *NCCOCCC(*)=O]

In some embodiments, L1 is:

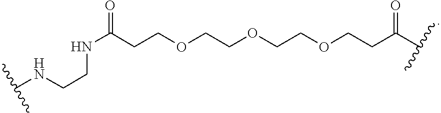 [SMILES: *NCCNC(=O)CCOCCOCCOCCC(*)=O]

Additional embodiments include those listed below.

In some embodiments, B is selected from the group consisting of:

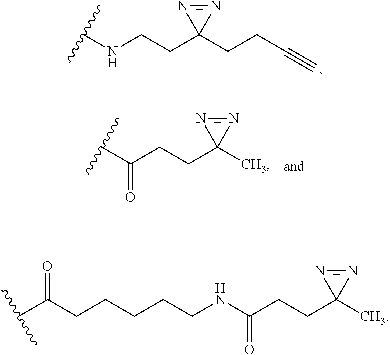 [SMILES: *NCCC1(CCC#C)N=N1 ; *C(=O)CCC1(C)N=N1 ; *C(=O)CCCCCNC(=O)CCC1(C)N=N1]

In some embodiments, B is:

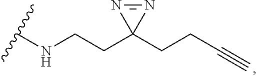 [SMILES: *NCCC1(CCC#C)N=N1]

In some embodiments, B is:

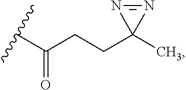 [SMILES: *C(=O)CCC1(C)N=N1]

In some embodiments, B is:

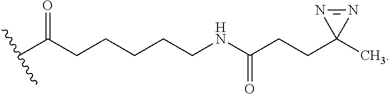 [SMILES: *C(=O)CCCCCNC(=O)CCC1(C)N=N1]

Additional embodiments include those listed below.

In some embodiments, psoralen is:

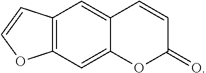 [SMILES: O=c1ccc2cc3ccoc3cc2o1]

In some embodiments, benzophenone is:

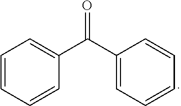 [SMILES: O=C(c1ccccc1)c1ccccc1]

In some embodiments, 4′,6-diamidino-2-phenylindole (DAPI) is:

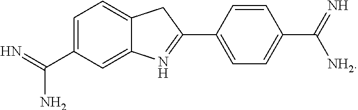 [SMILES: N=C(N)c1ccc(C2=Nc3cc(C(=N)N)ccc3C2)cc1]

In some embodiments, non-limiting examples of a Hoechst dye(s) and salts thereof include:

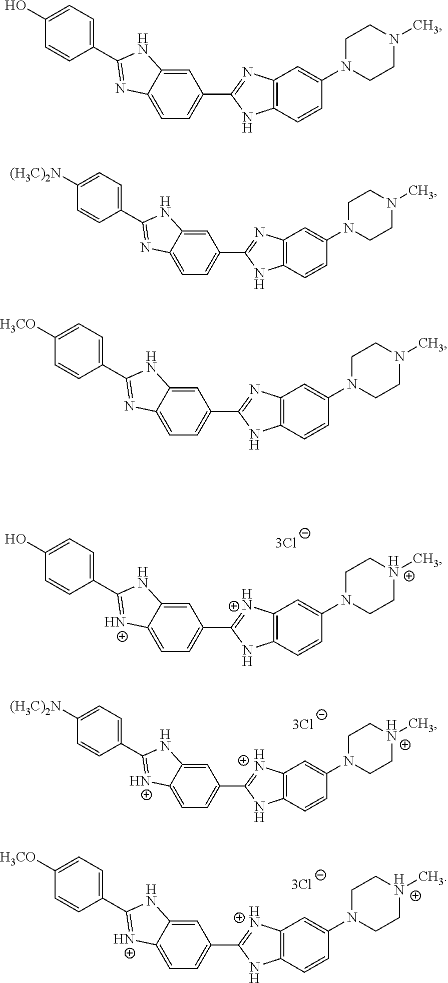

In some embodiments, non-limiting example of a G quartet binding molecule amine derivative:

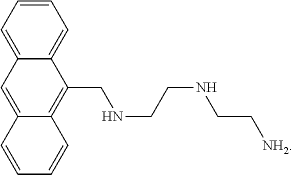 [SMILES: NCCNCCNCc1c2ccccc2cc2ccccc12]

In some embodiments, 4′-aminomethyltrioxsalen (4AMT) is:

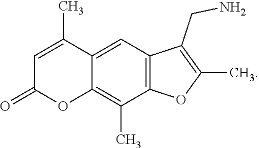 [SMILES: Cc1oc2c(C)c3oc(=O)cc(C)c3cc2c1CN]

In some embodiments, methyltrioxsalen is:

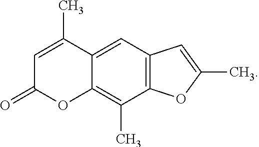 [SMILES: Cc1cc2cc3c(C)cc(=O)oc3c(C)c2o1]

Additional embodiments include those listed below.

In some embodiments, 4AMT-LC-SDA is:

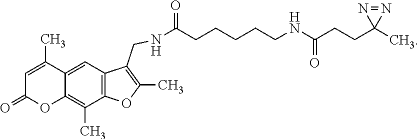 [SMILES: Cc1oc2c(C)c3oc(=O)cc(C)c3cc2c1CNC(=O)CCCCCNC(=O)CCC1(C)N=N1]

In some embodiments, 4AMT Hexenedioate AAD is:

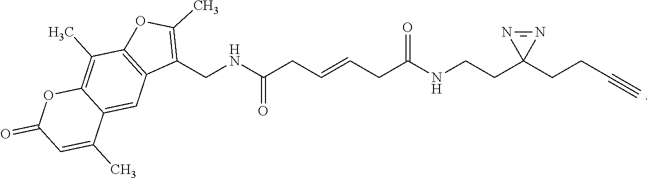 [SMILES: C#CCCC1(CCNC(=O)C/C=C/CC(=O)NCc2c(C)oc3c(C)c4oc(=O)cc(C)c4cc23)N=N1]

In some embodiments, SPB-Spermidine-AD is:

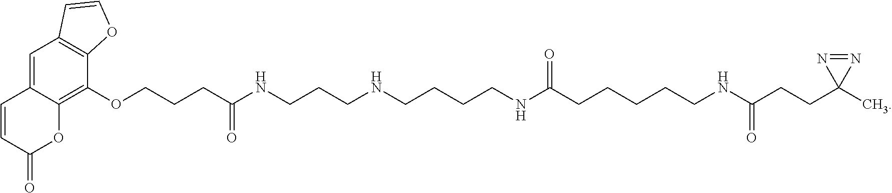 [SMILES: CC1(CCC(=O)NCCCCCC(=O)NCCCCNCCCNC(=O)CCCOc2c3occc3cc3ccc(=O)oc23)N=N1]

In some embodiments, SPB-PEG4-ADD is.

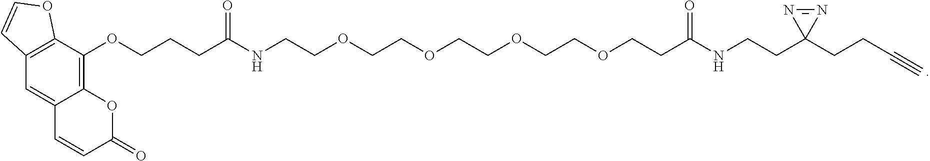 [SMILES: C#CCCC1(CCNC(=O)CCOCCOCCOCCOCCNC(=O)CCCOc2c3occc3cc3ccc(=O)oc23)N=N1]

In some embodiments, 4AMT-SDAD is:

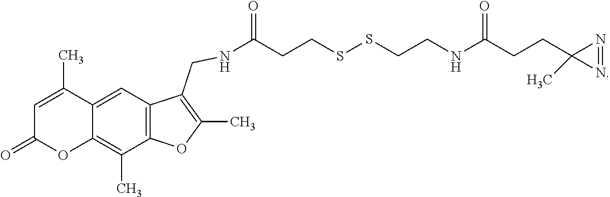 [SMILES: Cc1oc2c(C)c3oc(=O)cc(C)c3cc2c1CNC(=O)CCSSCCNC(=O)CCC1(C)N=N1]

In some embodiments, 4AMT Terephthalate AAD is:

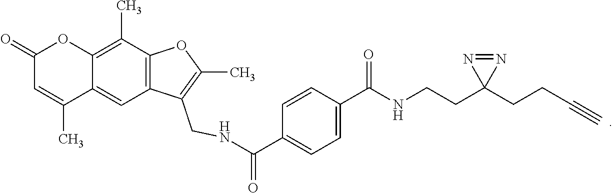 [SMILES: C#CCCC1(CCNC(=O)c2ccc(C(=O)NCc3c(C)oc4c(C)c5oc(=O)cc(C)c5cc34)cc2)N=N1]

In some embodiments, SPB-PEG3-AAD is:

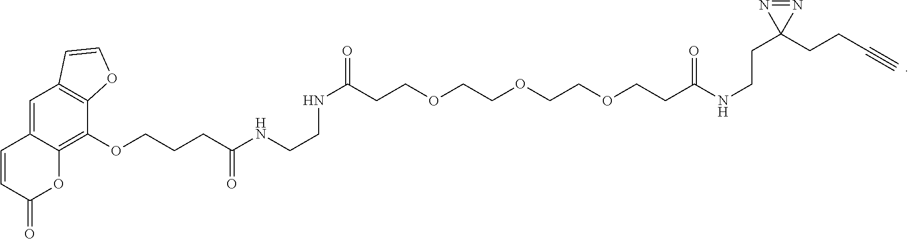 [SMILES: C#CCCC1(CCNC(=O)CCOCCOCCOCCC(=O)NCCNC(=O)CCCOc2c3occc3cc3ccc(=O)oc23)N=N1]

In some embodiments, SPB-AAD is:

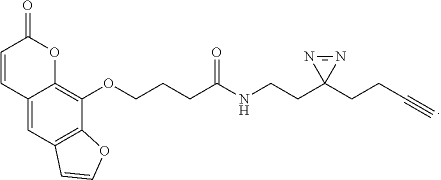 [SMILES: C#CCCC1(CCNC(=O)CCCOc2c3occc3cc3ccc(=O)oc23)N=N1]

Additional embodiments include those listed below.

In various embodiments, the present invention provides a method of crosslinking a nucleic acid with a protein in proximity in a system, comprising: incubating a compound of the present invention with the system, and irradiating the system with an ultraviolet light. In some embodiments, the system comprises a nucleic acid and a protein. In some embodiments, a compound of the present invention is a compound of Formula (I), a compound of Formula (II), a compound of Formula (III), a compound of Formula (IIa), a compound of Formula (IIb), a compound of Formula (IIc), a compound of Formula (IId), or a compound of Formula (IIe), or any combination thereof. In some embodiments, the system comprises a nucleic acid and a protein. In some embodiments, the nucleic acid is DNA, RNA, or combination thereof. In some embodiments, the compound of the present invention is a compound of Formula (II). In some embodiments, the method is performed in vitro, in vivo, or combination thereof. In some embodiments, the method is performed in vitro or in vivo. In some embodiments, the method is performed in vitro. In some embodiments, the method is performed in vivo. In some embodiments, the nucleic acid and protein are in proximity to one another in the system. In some embodiments, the system is a biological system. In some embodiments, the system is a live biological cell. In some embodiments, the live cell is a live biological cell. In some embodiments, the system is a live mammalian cell.

In various embodiments, the present invention provides a method of crosslinking a nucleic acid with a protein in a system, comprising: providing a compound of the present invention; providing a system, wherein the system comprises a nucleic acid and a protein; contacting the compound with the nucleic acid and the protein in the system; and irradiating the system with an ultraviolet light under conditions effective to crosslink the nucleic acid with the protein. In some embodiments, the system is a live cell. In some embodiments, the system is an in vivo system. In some embodiments, the system is an in vitro system. In some embodiments, the system is an in vivo system or an in vitro system. In some embodiments, a compound of the present invention is a compound of Formula (I), a compound of Formula (II), a compound of Formula (III), a compound of Formula (IIa), a compound of Formula (IIb), a compound of Formula (IIc), a compound of Formula (IId), or a compound of Formula (IIe), or any combination thereof. In some embodiments, the system comprises a nucleic acid and a protein. In some embodiments, the nucleic acid is DNA, RNA, or combination thereof. In some embodiments, the system is a sample. In some embodiments, the system is a biological sample. In some embodiments, the compound of the present invention is a compound of Formula (II). In some embodiments, the method is performed in vitro, in vivo, or combination thereof. In some embodiments, the method is performed in vitro or in vivo. In some embodiments, the method is performed in vitro. In some embodiments, the method is performed in vivo. In some embodiments, the nucleic acid and protein are in proximity to one another in the system. In some embodiments, the system is alive biological cell. In some embodiments, the live cell isa live biological cell. In some embodiments, the system is a live mammalian cell.

In various embodiments, the present invention provides a method of crosslinking a nucleic acid with a protein in a sample, comprising: providing a compound of the present invention; providing a sample, wherein the sample comprises a nucleic acid and a protein; contacting the compound with the nucleic acid and the protein in the sample; and irradiating the sample with an ultraviolet light under conditions effective to crosslink the nucleic acid with the protein. In some embodiments, the sample is a live cell. In some embodiments, the sample is an in vivo sample. In some embodiments, the sample is an in vitro system. In some embodiments, the sample is an in vivo sample or an in vitro sample. In some embodiments, the sample is a biological sample. In some embodiments, a compound of the present invention is a compound of Formula (I), a compound of Formula (II), a compound of Formula (III), a compound of Formula (Iia), a compound of Formula (Iib), a compound of Formula (IIc), a compound of Formula (IId), or a compound of Formula (Iie), or any combination thereof. In some embodiments, the sample comprises a nucleic acid and a protein. In some embodiments, the nucleic acid is DNA, RNA, or combination thereof. In some embodiments, the compound of the present invention is a compound of Formula (II). In some embodiments, the method is performed in vitro, in vivo, or combination thereof. In some embodiments, the method is performed in vitro or in vivo. In some embodiments, the method is performed in vitro. In some embodiments, the method is performed in vivo. In some embodiments, the nucleic acid and protein are in proximity to one another in the sample. In some embodiments, the sample is a live biological cell. In some embodiments, the live cell is a live biological cell. In some embodiments, the sample is a live mammalian cell.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

The purpose and applications for the disclosed probes are wide range, including examples of: i) as a more efficient and effective crosslinker replacement of formaldehyde or similar aldehyde reagents for in vitro or in vivo crosslinking, to explore nucleic acid situations upon proximity proteins, such as in immuno precipitation (IP), chromatin precipitation (ChIP), 3D chromatin conformation capture techniques (HiC/HiChIP); ii) as a crosslinker in exploring protein binding situations on nucleic acids in proteomic analysis such as protein or peptide identification of mass spectrometry or electrophoresis, since psoralen head from the probe can be released from the nucleic acids by irradiation with different wavelength (around 230 nm), and all other different nucleic acids binding heads can be reversed through the aforementioned cleavable azo junction (FIG. 3A-FIG. 3C). The remaining part still clung to the protein side after cleavage could be used as a unique mass ID/fingerprint in spectrum comparison. Isotoped compound with the same design can also be used as IDs.

Compared to the traditional protein nucleic acid crosslinkers such as formaldehyde or lengthy ones such as DSG (disuccinimidyl glutarate) or DSSP (3,3′-dithiobis(sulfosuccinimidyl propionate)), the probes disclosed herein has following advantages: i) it gives a direct, length controllable (through different length of NHS or alkyne linker cores), flexible and water soluble (using PEG core) crosslinking result between nucleic acids and proteins. In comparison, formaldehyde derivatives rely binding of these two through indirect ways of protein to nucleic acids tightly bound proteins, rather than directly onto nucleic acids themselves: ii) This direct way of binding would give a more precise protein binding location on nucleic acids either genome wide or locally labeled by fluorophores. In comparison, the traditional formaldehyde-based approach relies on the distribution, density and the location of nucleic acids tightly bound helper proteins such as histones, which result in imprecision evaluation of chromatin protein binding results. iii) The multi-arm PEG core can give the probe with multiple other desired functions and could be assembled in a relatively easy combinatorial way, such as aforementioned enrichment handle, reversibility through cleavage, length control, fluorescence, etc. iv) Compared to formaldehyde that can crosslink all other proteins of everywhere and thus result in distorted proximity reality and denatured epitope for antibody to recognize, our designed probes, as performed through direct DNA crosslink and length control will not result in any of these. Protein-protein crosslink cannot take place in large quantity because of probe's nucleic acids specific recognition end, and therefore the epitope from the rest of the parts of the protein is much more preserved in its native form. v) The photo chemical groups selected for the new probes described herein have the similar long wavelength UV spectrum (around 300 to 360 nm), compared to shorter UV direct crosslinking without any probe, our probe has less energy damage towards crosslinking biomolecule targets. And probe including design makes it much more efficient in crosslinking between nucleic acids and proteins.

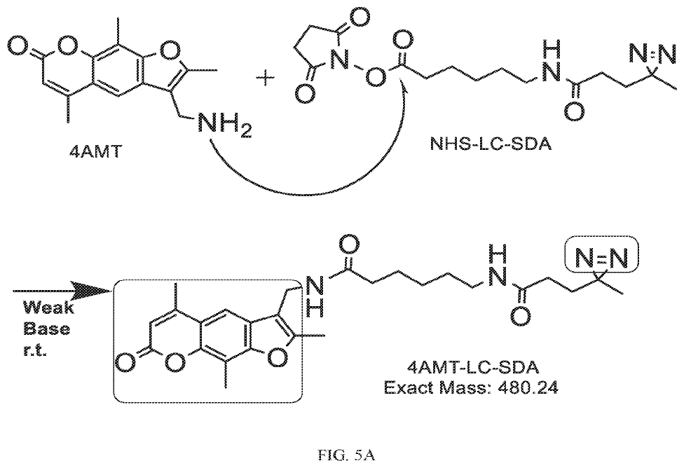

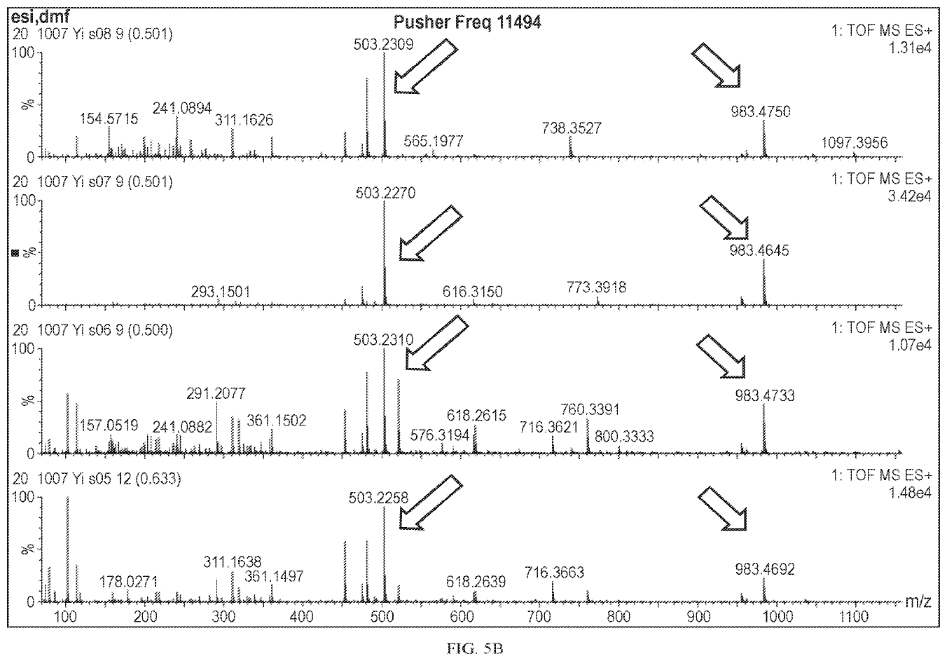

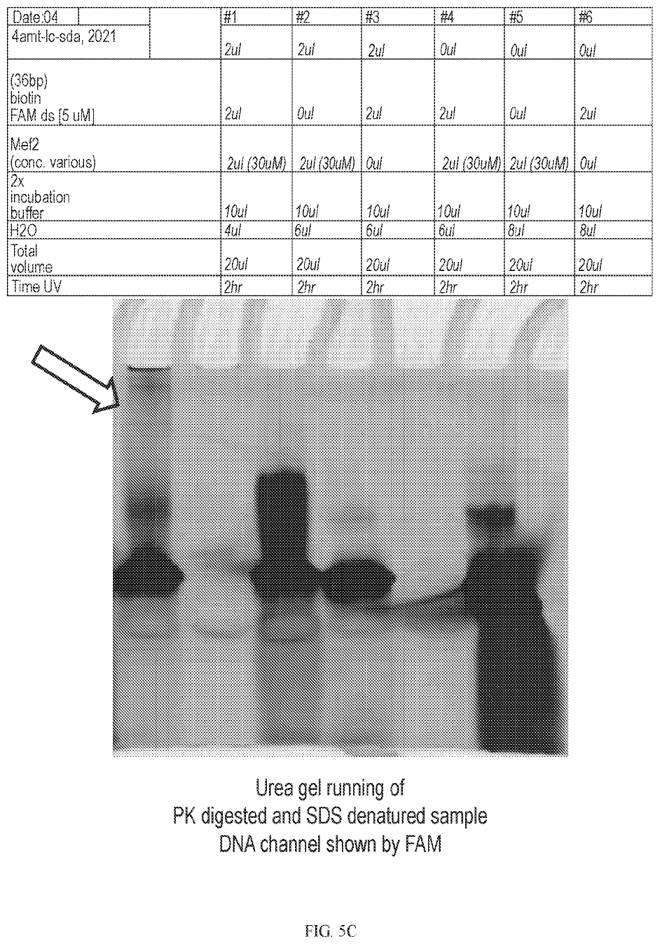

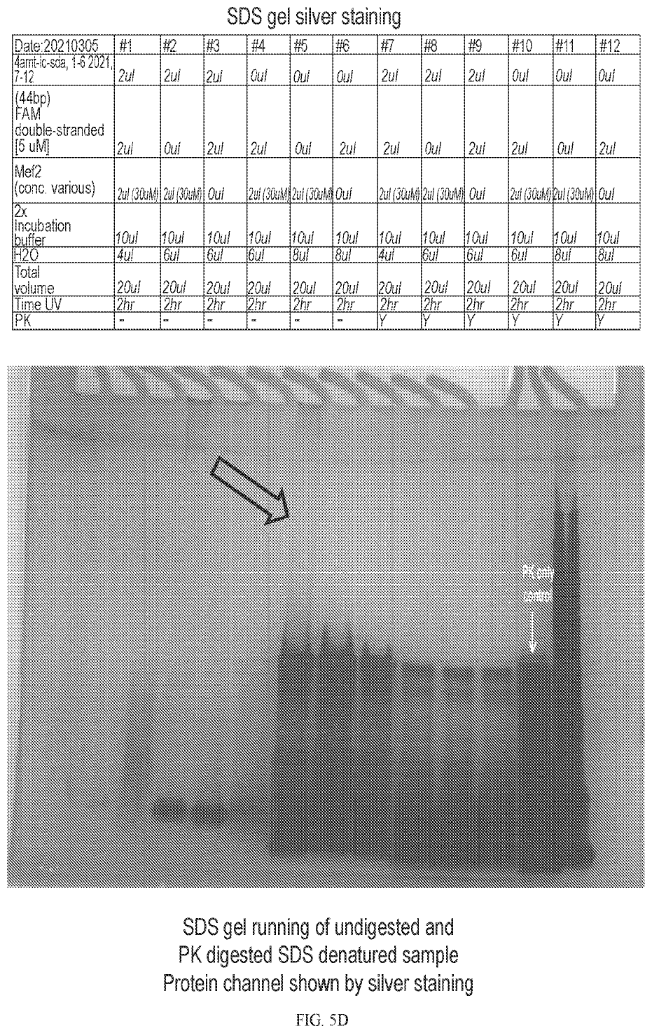

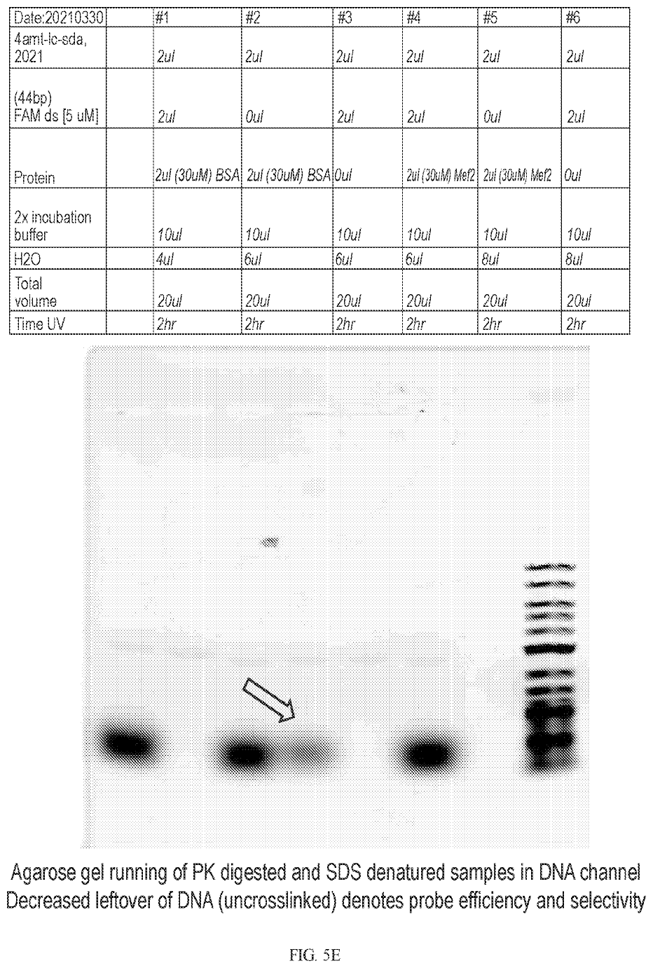

In vitro assays have confirmed the success and the efficiency of the demo probe (AMT-LC-SDA) in crosslinking double strand DNA of various contents and DNA-binding protein human myocyte enhancer factor 2A (MEF2A) (1-95 aa) (FIG. 5C-FIG. 5E). The solid crosslinking is confirmed for the probe with UV photo crosslinking conditions by denaturing or digestive approaches such as running with 8.3 M urea gel, SDS gel, combined with sample with overnight digestion of proteinase K at 65 Celsius and denatured at high temperature of 95 Celsius with SDS loading dye. Compared with all control conditions, only when probe and UV are both present, the crosslinking of protein and nucleic acids takes place. Under these harsh evaluation conditions, the protein got digested on the crosslinking complex thoroughly, yet the remaining crosslinked amino acids residuals still have made the DNA to be in a larger size and shifted clearly compared to other controls, which is indicated by the bold shaped arrow. Agarose gel was also used for its bigger pore size to estimate the crosslinking efficiency (UV 2 hrs), estimated by the amount of the left free DNA, our probe has reached around 30-60% efficiency (FIG. 5E), which is much higher than the UV-initiated protein DNA crosslinking without the use of a probe (10-20%) as reported in Nature Communications, volume 11, Article number: 3019 (2020). Additionally, the probe showed prominent selectivity for the nucleic acid binding protein: even in the presence of a high concentration of random non-nucleic acid binding protein such as BSA, only MEF2A sample shows consumption of DNA substrate by crosslinking (FIG. 5C-FIG. 5E). This further supports the idea of applying this probe for replacement of formaldehyde, it crosslinks the protein only within the proximity of nucleic acids, thereby largely reducing the random protein-protein crosslinking noise and helps to preserve the native state of protein epitopes.

### Example 2

Compared with the existing crosslinking technologies, the probes to be developed by the proposed research will have the following features: (1) By using DNA binding molecules, we can achieve regio-selectivity of photochemical reactions at or near the DNA binding site, thereby reducing the background noise of non-specific crosslinking. Unlike formaldehyde which will react with any proteins in the cells, causing damage to antibody epitopes and DNA-binding surface of TFs, our probes (for example, psoralen 4AMT head) will preferably bind only to nucleic acids by its nucleic acids specific recognition head (FIG. 2A, FIG. 2B). (2) By synthetic modification, we can introduce photo-affinity labeling groups that are highly efficient and can be activated by long wavelength UV (350 nm), thereby reducing the photo damage associated with the use of large doses of short UV (250 nm) irradiation. The synthetically introduced photo-affinity label (such as diazirine) will be a much more potent photo crosslinking group than endogenous protein residues and nucleoside bases. (3) Versatility in synthetic introduction of functional heads for customized applications (FIG. 2C). For example, multiple arms of protein photo-affinity labeling groups could be introduced to capture more than one TFs bound to composite DNA elements, thereby providing direct experimental evidence for transcriptional synergy of multi-TF complexes (as shown the NFAT-Fos-Jun ternary complex in FIG. 2C). The linker length could be varied to capture proximal DNA-binding domain or more distal protein cofactors recruited by the TF. Furthermore, the linker could be made cleavable so that the captured peptide (after the protease digestion) could be released for Mass Spectrometry analysis, thus permitting not only the identification of DNA sites (as done in ChIP assay) but also the structural mapping of DNA binding surface on protein (as in the XL-MS studies mentioned above).

Our initial attempt was to find a way to non-specifically functionalize DNA with a primary amine. For this purpose, we chose cell permeable, non-toxic 4′-aminomethyltrioxsalen (4AMT)(a psoralen derivative) that can bind and intercalate DNA nearly non-specifically (binding site preference 5′-TA>5′-AT>>5′-TG>5′-GT′), and under long wavelength (˜360 nm) UV illumination, crosslink to DNA covalently with high efficiency (up to 80%), thereby introducing an efficient nucleophile to DNA. Subsequently, any bi-functional amine reactive groups such as DSS could be used to crosslink DNA to proteins bound nearby. While this design solved the problem of low DNA reactivity, the amine reactive crosslinking agents could still modify other cellular proteins non-specifically to introduce high background noise and affect the recognition of TF by antibody, and also perhaps destroy the DNA binding surface (see FIG. 5G and description below). We therefore sought to use DNA-bound molecules to limit crosslinking reactions only to proteins that are bound or recruited to DNA. These considerations led to the design of the bi-functional photo-crosslinking probes.

The invention includes design and custom-synthesis of a class of new molecular tools that have at least two photo-crosslinking functional groups, hence the name bi-functional photo-crosslinking (BFPX) (FIG. 2C). One of the functional groups is responsible for binding and crosslinking to DNA under UV illumination, and the other is to capture proteins that are bound or recruited to DNA through UV-activated carbene or nitrene. The two functional groups are connected by a linker engineered to bear features that can facilitate the monitoring, isolation and analyses of the crosslinked protein-DNA complexes.

For the DNA binding and crosslinking head, a number of natural or synthetic DNA binding molecules, including psoralen or derivatives (Cimino et al., Anu. Rev. Biochem. 54, 1151-1193 (1985)), DAPI (Kapuscinski, Biotech. Histochem. 70, 220-233 (1995)), Hoechst dye (Carrondo et al., Biochemistry 28, 7849-7859 (1989)), which bind DNA nearly nonspecifically, could be used for genome-wide capture of all protein/DNA complexes. (Psoralen binds preferentially to nucleosome free regions of the genome, which might be a desired feature for studying protein complex active chromatin regions.) Specific DNA binding molecules, such as sequence-specific DNA binding polyamides (Nickols et al., Proc. Natl. Acad. Sci. U.S.A 104, 10418-10423 (2007)), or G quartet binder, could be used for targeted capture of protein-DNA complexes bound to a specific genomic locus. For the protein capturing head, we can use a photo-affinity labeling group diazirine (Mackinnon et al., Curr. Protoc. Chem. Biol. 1, 55-73 (2009)). Diazirine has a long UVA wavelength activation spectrum (340-365 nm) Similar to psoralen. The protein capturing head can be a single or multiple arms of diazirine for crosslinking one or more proteins bound to DNA near the psoralen insertion sites. The two heads are connected through a synthetic linkage of variable lengths. A shorter linker will be more efficient in capturing proximal DNA-binding domains of TFs, and a longer linker will be more efficient in capturing distal activation/repression domains or co-factors. Varying the arm length can also help improve capturing efficiency and selectivity by reducing crosslinking back to DNA or to faraway non-specific proteins. Two exemplary ways are choosing a different multi-arm core or linking diamine of various lengths to the protein capture head. The linker core can be a commercially available multi-PEG arm cyclooctyne linkage molecule (Creative PEG works). The arm composed of PEG is water soluble. The Copper free clickable DBCO end makes it bioorthogonal and assembly efficient that can be used for extraction enrichment handle, fluorescent labelling add-on, or cleavable MS index to the peptide. The flexibility of the linker can also be modulated by introducing unsaturated moieties (such as double bonds, triple bonds) to reduce back crosslinking to DNA hence favoring crosslinking to proteins bound to the DNA. Examples of double-bond linkers are shown in FIG. 3D. A general design using a commercially available multi PEG arm cyclooctyne linkage molecule (Creative PEG works) is depicted in FIG. 3B.

Preliminary studies: The major goal of developing BFPX is to allow for direct crosslink between DNA and its binding proteins. This feature is evaluated first by in vitro assembled TF/DNA complexes with purified TF proteins and synthetic DNA substrates, which could simplify the analysis of the crosslinked products and help quantify the crosslinking yield. This result in turn could facilitate the optimization of the probes and protocol design.

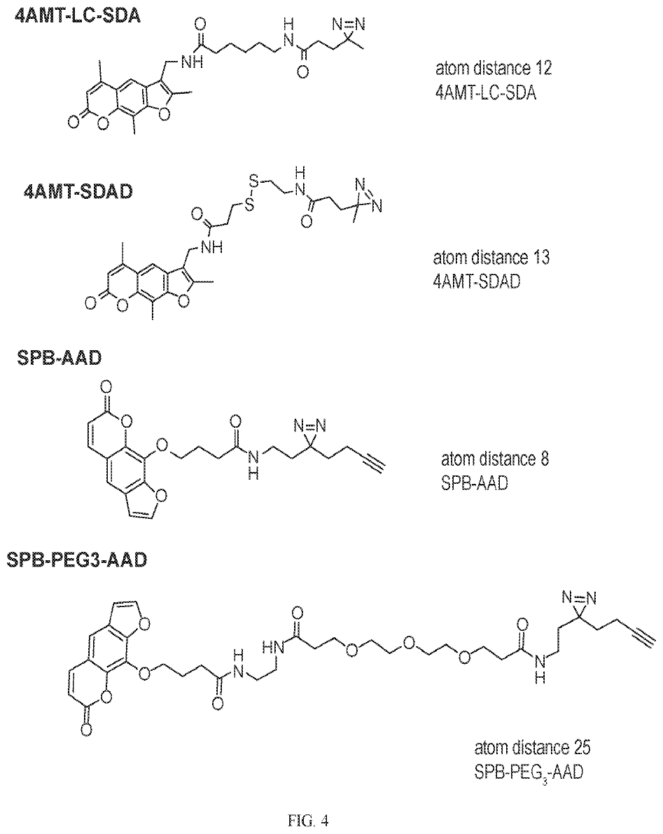

In preliminary studies, we have successfully synthesized four exemplary psoralen-based BFPX probes: 4AMT-LC-SDA, 4AMT-SDAD, SPB-AAD, and SPB-PEG3-AAD (FIG. 4). Psoralen is a plant natural product that has excellent cell permeability and tolerance. It shows little reactivity toward proteins but binds (intercalates) preferably to double-stranded DNA and RNA with μM (Kd) affinity. Upon UVA activation (˜360 nm), it forms stable covalent adduct with DNA and RNA. These properties of psoralen favor its use in in situ studies of chromatin structures and protein-nucleic acid interactions. In various embodiments of the present invention, we conceive that the preferential binding of psoralen to open chromatin regions makes it a great choice for designing BFPX probes to capture transcription factors, as transcription factors are known to bind almost exclusively to open chromatin regions. The general strategy of BFPX is applicable to other DNA binding heads that bind genome either non-specially or specifically. Using commercially available psoralen and diazirine derivatives, we synthesized a number of BFPX probes with various linker design features (FIG. 4). In addition to 4AMT, we also extended the BFPX probe synthesis to other psoralen derivatives, such as SPB (succinimidyl-[4-(psoralen-8-yloxy)]-butyrate).

### 4AMT-LC-SDA

The BFPX probe 4AMT-LC-SDA was synthesized with good yield by the NHS-amine chemistry using psoralen and diazirine derivatives (FIG. 5A, FIG. 5B). To test its photo crosslinking ability, human transcription factor MEF2A (the MADS-box/MEF2 domain) and double strand DNA containing a MEF2 binding site were used. We first used electrophoresis mobility shift assay (EMSA) to validate the activity of the purified recombinant MEF2A DNA-binding domain. After finding out the optimal protein to DNA ratio, the in vitro assembled MEF2/DNA complex was then subject to photocrosslinking in the presence or absence of the 4AMT-LC-SDA probe with or without UV illumination. Multiple other control experiments were carried out in parallel to test the specificity of photocrosslinking between the DNA and MEF2. Specifically, parameters such as protein/DNA amounts (1:1 to 20.1), ratio, UV time (10 m to 4 hrs), wavelength (320 to 360 nm), LV source (mercury long lamp, LED) and their distance to the reaction mixture have been optimized along the course. The MEF2 Protein and DNA were incubated first to mimic an in vivo binding mode and stoichiometry, and then the mixture was incubated with our 4AMT-LC-SDA probe to undergo UV illumination. The results were evaluated by running denaturing gels (SDS in FIG. 5D, or Urea in FIG. 5C) against the high temperature denatured reaction mixtures and checking the respective protein (silver stain) or DNA (FAM label) signals. To further rule out any other possible false positiveness during loading, proteinase K digestion was applied to all the samples including the negative controls with SDS at 65 degrees.

The results indicated that only when MEF2A, DNA, the BFPX probe (4AMT-LC-SDA) and UV irradiation are present could we have a successful photo crosslink complex between protein and DNA. Furthermore, non-DNA binding control protein BSA under the same condition did not show any signs of forming crosslink with the DNA, demonstrating the high selectivity of our 4AMT-LC-SDA probe for targeting only DNA binding proteins. By using the agarose gel for separating large complexes, we compared the intensity of bands corresponding to various molecular species, the photo crosslinking efficiency of the MEF2-DNA complex under this current condition is estimated to be around 50%.

We further tested the activity of this probe using in vitro assembled transcription factor/DNA complexes, wherein two human transcription factors, myocyte enhancer factor 2 (MEF2; herein we used MEF2A) and nuclear factor of activated T cells (NFAT; herein we used NFAT1), and their respective double-stranded DNA substrates labeled with fluorescein (5′6-FAM), were used for the in vitro assay.

We used electrophoresis mobility shift assay (EMSA) to examine the effect of formaldehyde (FA), a BFPX probe (4AMT-LC-SDA), and UVA (at 365 nm) on the DNA binding by NFAT1. A number of important observations are made from FIG. 5G. First, while formaldehyde did not show any effect on DNA alone (compare lane 2 and lane 1), it severely diminished the DNA binding by NFAT1 (compare lane 4 and lane 3) (the upward streaking bands likely represent residual NFAT/DNA complexes modified by formaldehyde outside the DNA binding surface). This observation is consistent with the notion that formaldehyde can modify the lysine-rich DNA binding surface of TFs and inhibit their DNA binding activity. By contrast, 4AMT-LC-SDS with and without UV365 or UV365 alone did not affect NFAT1 protein (data not shown). Second, UVA (365 nm), 4AMT-LC-SDA, and their combination did not affect the DNA mobility (compare lanes 5, 6, and 7) and the DNA binding by NFAT1 (compare lanes 8, 9, and 10). The presence of both DNA and protein in the same shifted band can be checked by the FAM fluorescence signal (top) and Coomassie stain (bottom), respectively (free NFAT is positively charged and not observable under the EMSA condition). Finally, when the sample of lane 10 is run on a denaturing SDS gel, a covalent complex containing both protein and NFAT can be observed (see FIG. 5H, lanes #1, 2, 3).

Through preliminary studies like these, we have found that the psoralen-based BFPX probes (FIG. 4) do not show any detectable effect on NFAT1/DNA binding at up to 150 μM concentration, which is consistent with previous in vivo studies showing that psoralen did not affect the endogenous transcription complexes at similar concentrations.

### 4AMT-SDAD

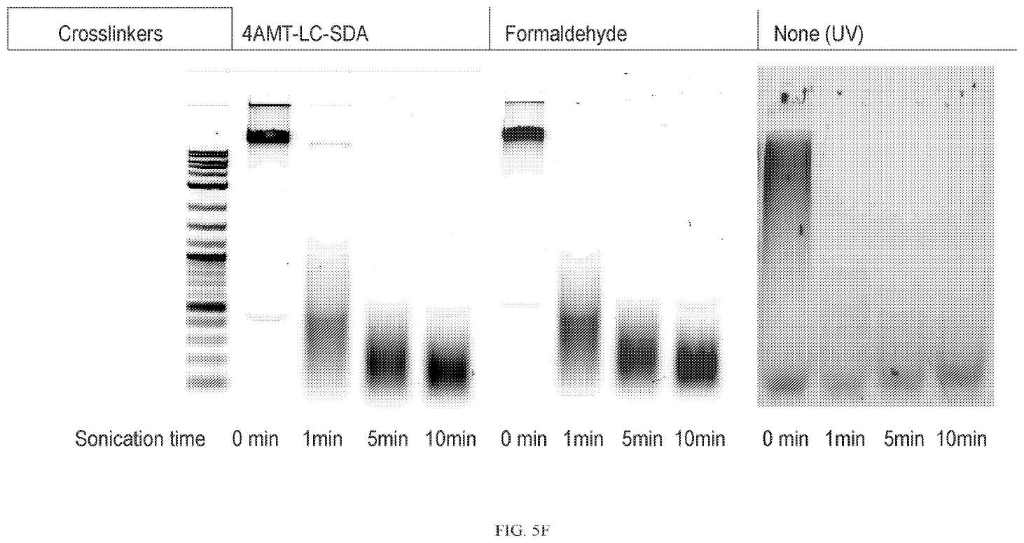

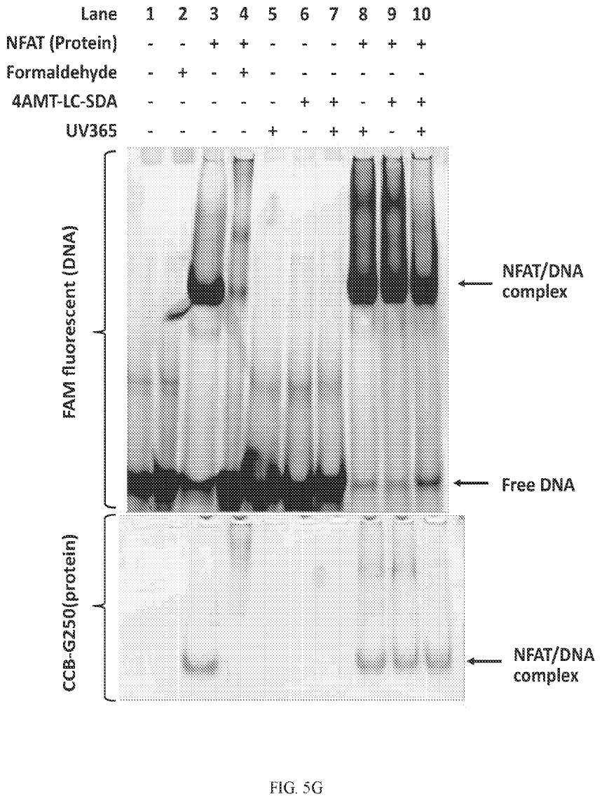

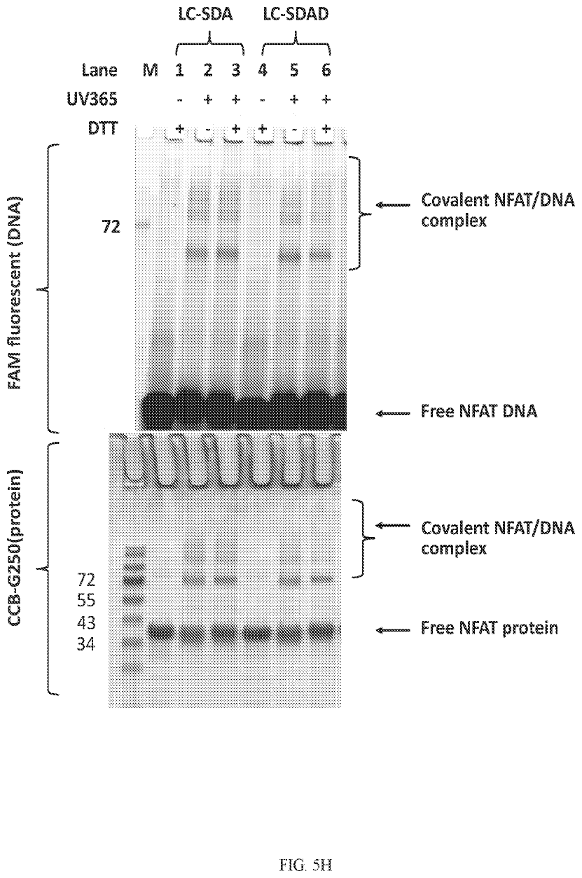

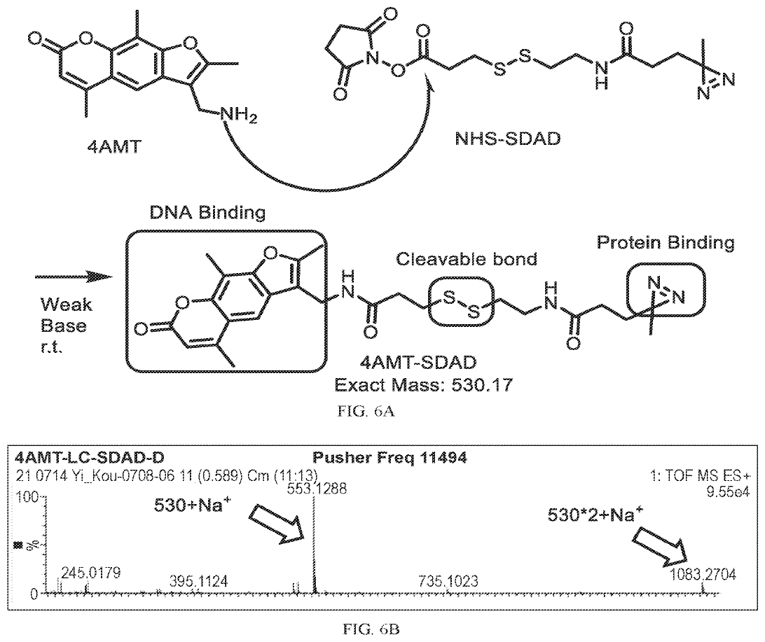

The BFPX probe 4AMT-SDAD with a cleavable link can greatly facilitate mass spec analysis of peptides released from crosslinked protein-DNA complexes after specific protease digestion. See FIG. 6A, FIG. 6B.

### SPB-AAD

We next used SDS denaturing gel to check for the formation of covalent protein-DNA complexes and determine the crosslinking efficiency, demonstrated with a BFPX probe, SPB-AAD. As shown in FIG. 7, for in vitro assembled MEF2A/DNA complex and the NFAT1/DNA complex, the addition of the BFPX probe (SPB-AAD) in the presence of UVA (365 nm) leads to the formation of complexes that run as larger molecular species than both free protein and DNA; and more importantly, these larger complexes contain both DNA and protein as indicated by the FAM signal and the coomassie stain (lanes 4-7 for the MEF2/DNA complexes and lanes 9-12 for the NFAT1/DNA complexes). The robust stability of these complexes under strong denaturing conditions (2% SDS and boiling for 10 min) is consistent with the covalent nature of the crosslinking reaction as designed in BFPX. By contrast, the controls (no treatment, lane 1; UV365 only, lane 2; and SPB-AAD only: lanes 3 and 8), showed only free protein and DNA dissociated under the denaturing condition. Surprisingly, even at a low probe concentration of 0.125 μM, a significant amount of crosslinked protein complex of MEF2/DNA (lane 7) and NFAT1/DNA (lane 12) were observed. As the probe concentration increased, more crosslinked protein/DNA complexes were formed, and the multiple bands likely represented protein-DNA complexes with various degrees of crosslinking, because multiple SPB-AAD molecules could bind the flanking region of the DNA binding sites and engage crosslinking reactions with the protein. At high probe concentrations (lane 5 and lane 10), the complexes become more uniform, likely due to the saturation binding of the BFPX probe to DNA. The reduced crosslinking efficiency at the highest probe concentration (lane 4 and lane 9) is due to the precipitation of the probe in an aqueous solution.

To estimate the crosslinking efficiency, we compared the fluorescence intensity of free DNA and DNA complexed with protein. We found that DNA (both the MEF2 DNA and NFAT1 DNA) did not show a significant mobility shift in SDS gel upon UV365 crosslinking in the presence of SPB-AAD and other BFPX probes we tested (data not shown). Moreover, almost all up-shifted DNA bands contain protein as indicated by the coomassie stain. Under protein excess conditions where most DNA is bound to protein, we estimated the crosslinking efficiency (the percentage of native protein-DNA complex being covalently crosslinked together) to be around 80-90% based on the free DNA FAM signal of lane 5 (vs lanes1-3) and lane 10 (vs lane 8).

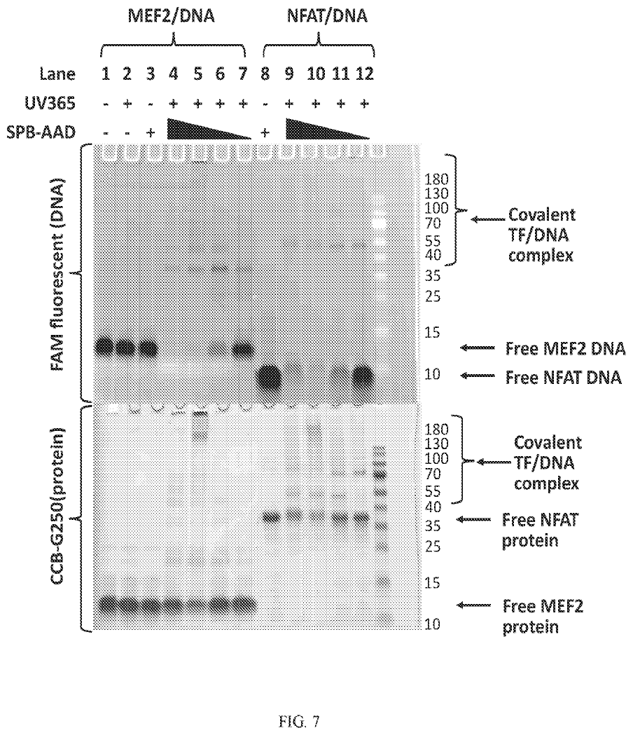

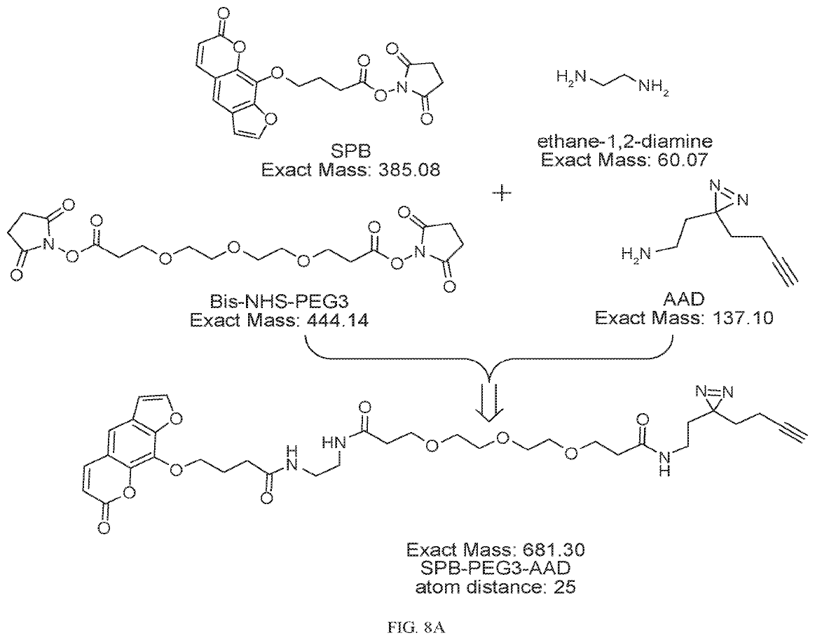

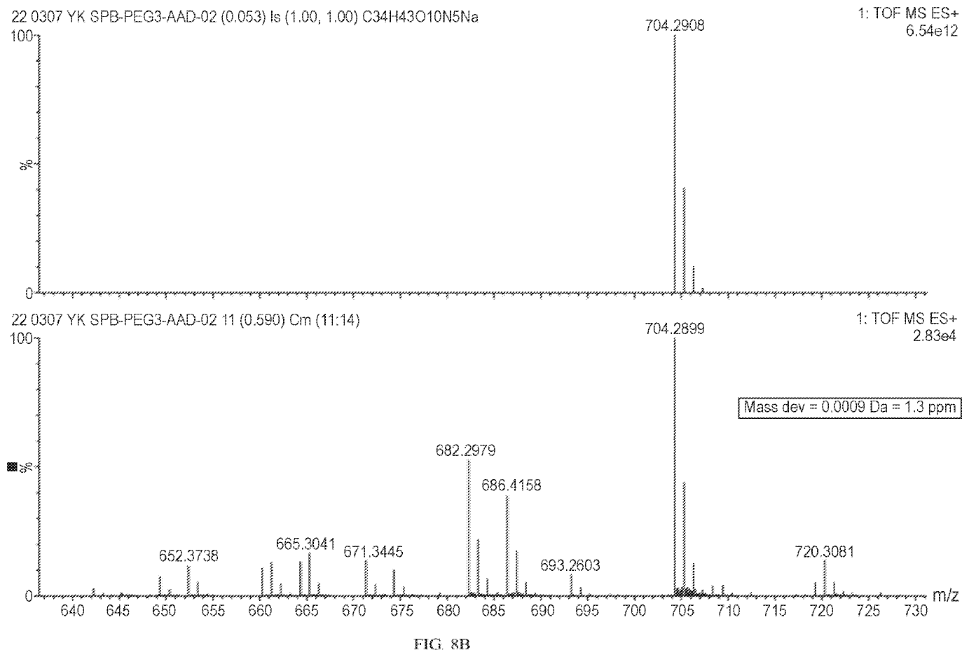

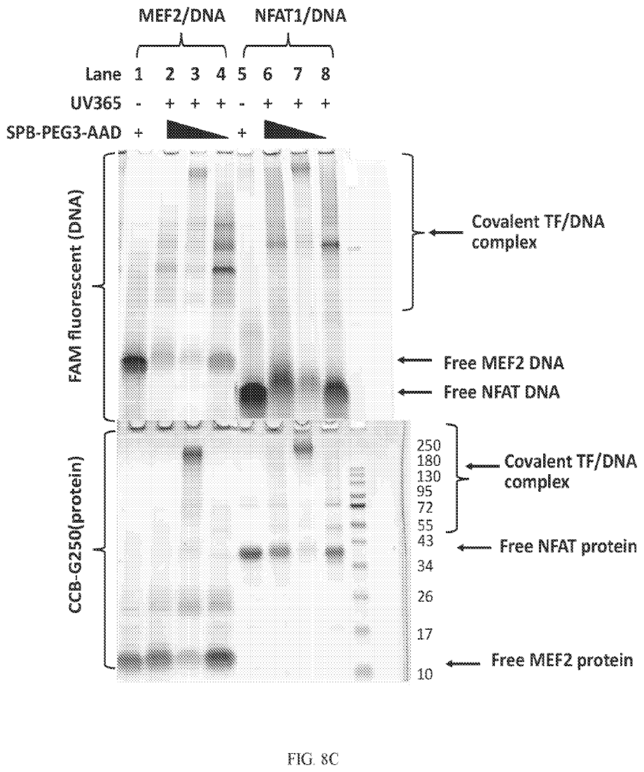

We have performed similar experiments with different BFPX probes and observed consistently highly efficient crosslinking of the MEF2/DNA complex and the NFAT1/DNA complex using the probe of 4AMT-LC-SDA (FIG. 5H, Lane #1, 2, 3) and 4AMT-LC-SDAD (FIG. 5H, Lane #4, 5, 6; also denoted as “4AMT-SDAD” in FIG. 4) and SPB-PEG3-AAD (FIG. 8C).

As shown in FIG. 5H, 4AMT-LC-SDA crosslinked NFAT to its DNA substrate efficiently (compare lanes 2 and 3 with lane 1). Similarly, 4AMT-LC-SDAD crosslinked NFAT to its DNA substrate efficiently (compare lanes 5 and 6 with lane 4). DTT treatment showed little effect on the NFAT-DNA complex crosslinked by 4AMT-LC-SDA (compare lane 2 and lane 3). By contrast, DTT treatment reduced significantly the NFAT/DNA complex crosslinked by 4AMT-LC-SDAD (compare lane 6 and lane 5). However, not all the covalent NFAT-DNA complexes is dissociated by the DTT treatment. It is possible that the tested reducing condition was not strong enough to reduce the disulfide bond in the 4AMT-LC-SDAD and a much stronger reducing reagent and/or condition may fully release the crosslinked the covalent NFAT/DNA complexes. Alternatively, a carbene based photo-crosslinking reaction may result covalent linkage between protein and DNA that is not cleavable by reducing reagents.

### SPB-PEG3-AAD

The BFPX probe SPB-PEG3-AAD has a longer linker arm that can facilitate the crosslinking to protein domains further away from the protein-DNA binding interface. See FIG. 8A, FIG. 8B.

For the BFPX probe SPB-PEG₃-AAD, when the probe concentration was sufficiently high (lane 3 and lane 7 of FIG. 8C), the crosslinking efficiency was more than 90% for the MEF2/DNA complex based on the DNA signal (comparing lane 3 and lane 1 in the 5FAM gel, the free DNA in lane 3 was nearly all gone as compared the strong band in lane 1). This is consistent with the protein signal (compare lane 3 and lane 1 in the CCB-250 stained gel, the free protein in lane 3 was very faint as compared with the strong band in lane 1). For the NFAT/DNA complex, a similar high crosslinking efficiency was observed (comparing lane 7 and lane 5 in the 5FAM gel, the free DNA in lane 7 was nearly all gone as compared the strong band in lane 5). Again, the protein signal showed consistent result (comparing lane 7 and lane 5 in the CCB-250 stained gel, the free protein in lane 5 was nearly all gone as compared with the strong band in lane 5). When the probe concentration was too high (lane 2 and lane 6), the probe precipitated out of solution, resulting lower crosslinking efficiency.

These results demonstrated the possibility of engineering tunable molecular tools for efficient and robust capture of protein-DNA complexes.

### Identification of Covalent Attachment Site:

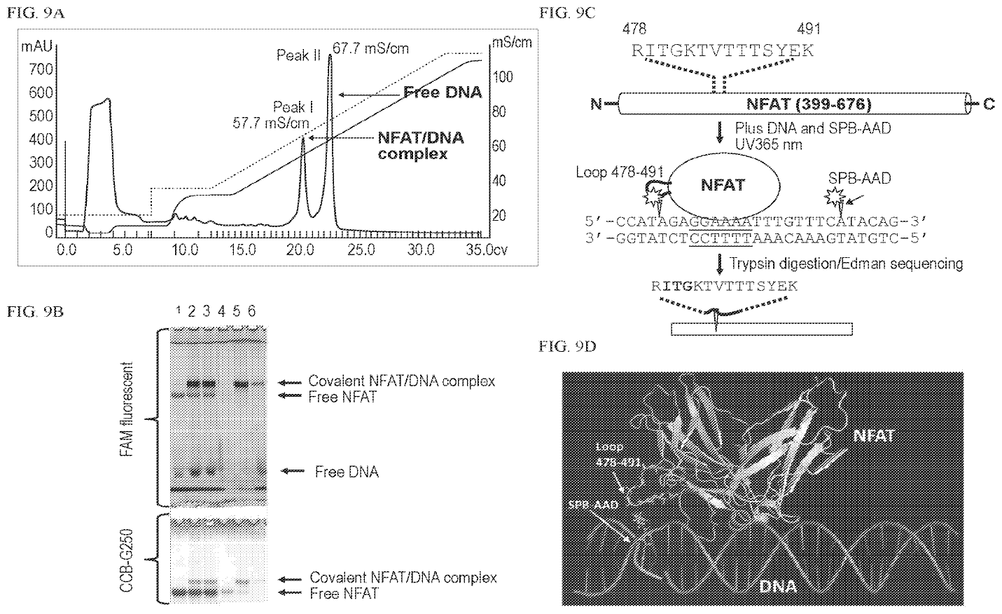

To further characterize BFPX mediated crosslinking reaction between protein and DNA, we identified the covalent attachment site on the protein. We first scaled up the crosslinking reaction corresponding to lane 11 of FIG. 7. We then purified the covalent-NFAT/DNA complex on FPLC using a mono-Q column (FIG. 9, panels a and b). The purified NFAT/DNA complex was digested with trypsin and subjected to another round of FPLC purification using Mono-Q. The DNA with a peptide attached to it was eluted like free DNA. The DNA-peptide conjugate was sent for Edman sequencing, which yield a unique sequencing motif Ile, Thr, and Gly that correspond to I479, T480, and G481 of human NFAT1 used in this study (FIG. 9, panel c). I479 is after R478, consistent with the fact the peptide is generated by trypsin digestion, which cuts at the C-terminus of Lys and Arg. Surprisingly, when this result was compared with the crystal structure of the NFAT/DNA complex, assuming that the psoralen head inserted into a 5′TpA-3′ in the DNA substrate used in the experiment and that the linker in SPB-AAD assumed an extended conformation, the diazirine head group pointed exactly at the loop harboring the loop of I479, T480, and G481(FIG. 9, panel d).

Despite the general belief that diazirine-generated carbene will react non-specifically with solvent molecules and DNA in addition to protein, given that there had been uncertainly of the DNA insertion activity of the modified psoralen head, our results have shown strongly that the psoralen head in the BFPX probe still retains a strong preference for binding to the TpA site despite the synthetic modifications, and that the carbene generated through UV activation of diazirine strongly favors crosslinking with proteins. If a large fraction of the carbene reacted with solvent molecules and DNA, then we would not observe the high crosslinking efficiency. If the high crosslinking efficiency was due to the many BFPX probes bound to DNA that crosslink to proteins, we would then not see a unique attachment site on NFAT. These observations demonstrate that BFPX can serve as a mechanistically clear, accurate and quantitative crosslinking technology for studying protein-nucleic acids interactions.

In various embodiments of the present invention, we further conceive to study: (i) BFPX protocol development using in vitro TF complex model systems: We will extend the above studies to other transcription factors that have been purified and studied by crystallography in the lab. These TFs include p53, FOS, Jun, TonEBP, FOXP2, FOXP3, GATA3, NF-kappaB p50, and NF-kappaB p65, NKX2.5. This group of TFs represents a variety of DNA binding domain families that can help us determine the general applicability of the BFPX approach. In addition to the direct DNA-binding proteins, we will also test if transcription cofactors recruited to DNA could be crosslinked to DNA using probes with longer linker lengths. For this, we will use the ternary TF complexes of MEF2/Cabin1/DNA complex, the MEF2/HDAC/DNA complex, and the MEF2/p300/DNA complex. These are classical higher-order TF complexes where transcription repressors (such as Cabin1 and class IIa HDACs) or activators (such as p300) are also recruited. Our structural studies of the TF/DNA complexes demonstrated above provide suitable model systems to develop and optimize the BFPX probes and the related protocols. Additionally, we will also map the atomic linkage between the protein and the DNA using the XL-MS approaches recently described for protein-DNA complexes captured by short UV (˜250 nm) and compare with our crystal structures. These studies will not only help cross-validate BFPX at the structural level, but will also establish a general approach to analyzing protein-DNA interactions using BFPX-based XL-MS. (ii) Further development of BFPX probes: multi-arm BFPX probes with variable linkers containing multi-functional features, including length, cleavability, enrichment handle, and fluorescent reporter.

Different linker lengths may be optimized for different experimental applications: shorter linker for immediate DNA-binding domains, and longer linker for recruited co-factors or faraway functional domains. A cleavable enrichment handle can also be added. The advantage is that upon purification of the crosslinked protein-DNA complexes, the proteins or their protease digested peptide fragments could be released by the cleavage of the linker to facilitate the enrichment and the subsequent protein analyses (e.g., LC/MS). We will also test if by introducing multiple diazirine protein capturing arms, we could increase the crosslink efficiency and enable capture of multiple nearby proteins. Via clickable core, various fluorescent labels can also be added to facilitate probe location tracking.

Another advantage of introducing multiple diazirine arms is to circumvent the need of introducing a photo-affinity label on DNA binding heads that do not have the intrinsic ability to undergo photo crosslinking with DNA like psoralen does, because one of the diazirine arms could serve as the crosslinking group for DNA and the other for DNA bound proteins. One general scheme for developing such multi-arm BFPX probes is outline in FIG. 3B. Since most compounds are commercially available or can be azidated mildly from amine (e.g. DAPI's primary amine), the assembly should be chemically facile. Considering it is a multi-step assembly, the desired compound will be purified (HPLC/silica gel chromatography) and confirmed by NMR in addition to mass spectrometry. The tests for photo-crosslinking for these multi-arm probes will be similar to the experiments of the BFPX probes described above in preliminary studies.

One such scheme for developing the multi-arm BFPX probe is described (FIG. 3B). Since most compounds can be azidated mildly from amine (e.g. DAPI's primary amine), the assembly should be chemically facile. Considering it is the multi-step assembly, the desired compound will be purified (HPLC/silica gel chromatography) and confirmed by NMR in addition to MS. The tests for photo-crosslinking for these multi-arm probes will be similar to the experiments of 4AMT-LC-SDA probe described above.

Some alternative considerations: psoralen is known to bind preferentially to nucleosome-free regions of the genome. While this might be a desired feature for studying protein complexes in active chromatin regions, this could also be a limitation of this class of probes if one wants to investigate protein-DNA interactions in other genomic regions such as the dense heterochromatin regions. For this reason, we will also extend our future BFPX probe design to other DNA binding molecules (such as DAPI and Hoechst dye) that have different DNA-binding properties than psoralen (FIG. 3B, upper half). When using DAPI and Hoechst dye as the DNA binding head to design BFPX probes, we will take advantage of the crystal structure of their complexes bound to DNA to pick the sites to introduce a linker with diazirine photo-affinity labelling groups (FIG. 3E).

### Applications of BFPX Probes in Cells

For this, we tested the prototype BFPX probe 4AMT-LC-SDA in the suspension GM12878 cell lines. The result showed that BFPX showed good crosslink effect as compared to the no-probe or no-UV controls (FIG. 5F).

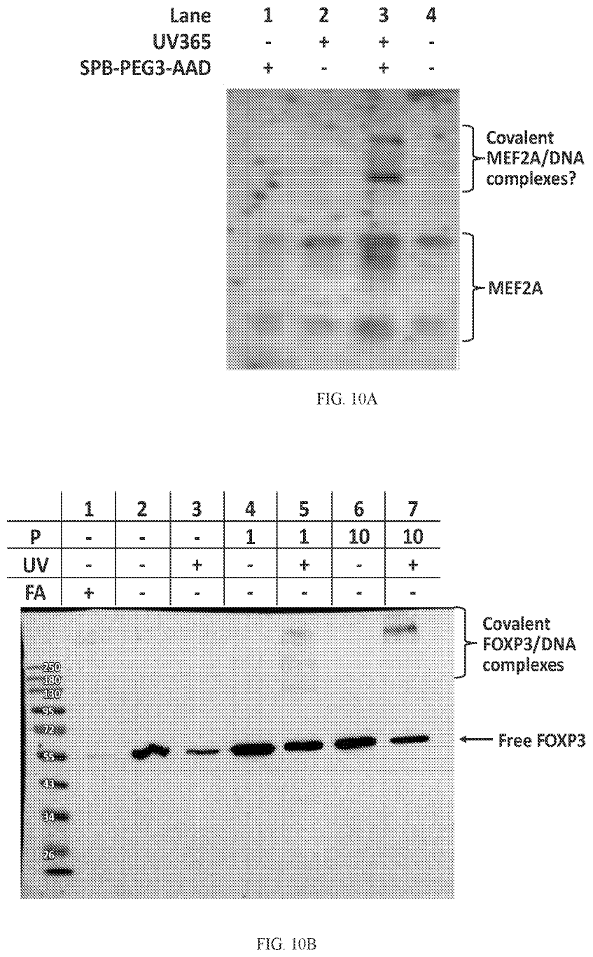

We tested the capture of MEF2A/DNA complexes in the GM12878 cell line, a Tier 1 ENCODE cell line with rich genomics data. As shown in FIG. 10A, the MEF2A antibody detected two bands in untreated GM12878 (lane 4), which is similar to what was shown for this antibody by the manufacturer (SCBT). While cells treated with only SPB-PEG3-AAD (lane 1) or only UV365 (lane 2) showed similar two bands of free MEF2A, the cells treated with SPB-PEG3-AAD showed two upper bands together with smearing bands, indicating that a mixture of larger MEF2A complexes were generated by UV365 induced crosslinking with SPB-PEG3-AAD. This initial testing indicated that UV365 and SPB-PEG3-AAD could capture MEF2 complexes in the nucleus.

We have performed similar experiments with a different system, Hela cells transfected with AVI-TEV-FLAG tagged FOXP3. Again, we observed that the BFPX probe SPB-AAD can capture higher molecular weight FOXP3 complexes (presumably covalent FOXP3-DNA complexes under UV 365 nm illumination. (FIG. 10B). This experiment shows that only when SPB-AAD and UV illumination are both present (lane 5 and 7), higher molecular weight FOXP3 complexes can be observed (compare lanes 5 and 7 with lanes 2, 3, 4 and 6). Furthermore, the formation of the higher molecular weight FOXP3 complexes is dosage dependent on the concentration of SPB-AAD (compare lane 7 and lane 5). Interestingly, the signal from the FA control lane (lane 1) is very weak, probably due to the trapping of FOXP3 in large protein complexes or the modification and/or masking of the antibody epitope.

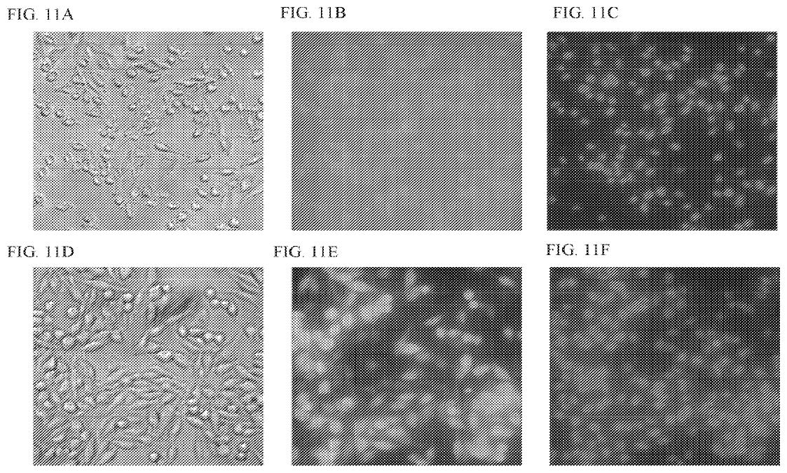

We also analyzed (i) cell permeability and subcellular distribution of the BFPX probes. We validated the cell and nuclear permeability of the BFPX probes and monitored their sub-cellular distribution using azide-containing fluorescent label through click reaction with the alkyne group (as in SPB-AAD and SPB-PEG3-AAD). Briefly, Hela cells were incubated with SPB-AAD (25 μM) in the dark for 30 min to allow the DNA binding and intercalation by the psoralen moiety of the BFPX probe. After washing off the excessive BFPX probe, the cells were illuminated with UV 365 nm for 5 min. An Alexa Fluor 647 picolyl azide molecule (from CLICK-IT™ Plus ALEXA FLUOR™ 647 Picolyl Azide Toolkit) was then used to react with the alkyne group on SPB-AAD in DNA via click reaction. After the click chemistry labeling, the unreacted fluorescent molecules were removed by washing. The nuclei were then analyzed using fluorescence imaging (FIG. 11). These experiments demonstrated that only when the BFPX probe was present and activated by UV crosslinking, the Alexa Fluor 647 picolyl azide molecule was immobilized in the cells via click reaction with the BFPX probe. It also shows that the BFPX probes can enter the cells and the nucleus efficient. This approach has been successfully used to monitor the nuclear binding and distribution of psoralen derivatives. We have previously used this approach to monitor the subcellular locations of alkyne-containing molecular tools (via STORM).

In various embodiments of the present invention, we also conceive to (ii) fine tune the BFPX-based crosslinking and extraction of TF/DNA complexes in cells. In addition to the endogenous MEF2A in GM12878, we have also selected two other model systems, MDA-MB-231 cells expressing N-3×FLAG-GATA3 (doxycycline-inducible) and HEK 293T cells expression AVI-TEV-FLAG-tagged FOXP3 (transient transfection). These additional systems will allow us to validate the general applicability of BFPX in capturing different TF/DNA complexes with different antibodies (FLAG) or tag (biotin) and at different abundance levels in cells. The formation of denaturing-resistant TF/DNA complexes will be monitored by SDS gel/western blot and further analyzed by nuclease (DNase I) and mass spectrometry. By monitoring the yield of covalent TF/DNA complexes from a given amount of cells (e.g., 1 million), we can fine tune the BFPX in vivo crosslinking protocol by varying the probe concentration and UV dosage. To facilitate a variety of subsequent analyses (e.g. ChIP-seq and proteomics), we will develop a protocol for preparative extraction of the covalent TF/DNA complexes from free proteins and DNA by modifying the existing phenol/toluene/chloroform extraction methods for covalent protein/DNA or covalent protein/RNA complexes.

In various embodiments of the present invention, we also conceive to put the BFPX probes in various (iv) test applications. We will apply BFPX in chromatin immunoprecipitation (ChIP) assays with sequencing (ChIP-seq) and Hi-C genomic analysis technique to compare with the formaldehyde in two suspension cell lines (K562, GM12878) and adherent (Hela) cell lines on four different TFs (CTCF, GATA1, PU.1, and MEF2A). These protein targets represent distinct classes of DNA binding domains, and their formaldehyde-based ChIP-seq data are available in all three cell lines, which will allow us to carry out a systematic comparison of the formaldehyde-based and BFPX-based ChIP-seq datasets. A number of metrics will be used to assess the performance of BFPX based ChIP-seq. We will first use data correlation between technical replicates to check the protocol reproducibility. We will also compare the ChIP-seq peaks of the same TF between different cells to see if significant differences (i.e. above the background noises defined by the technical replicates) can be observed and if yes, compare such differences with known signaling and gene expression differences between different cells. Such mechanistic analyses are often challenging if not impossible for formaldehyde ChIP-seq data of TFs due to the high background noises. As discussed earlier, a major and significant contributor of these noises is DNA fragments co-captured with the target TF targets via non-specific cross-linking of protein-protein complexes. By contrast, BFPX is designed to capture only (or at least preferably) proteins bound or recruited to DNA. We propose to use the percentage of isolated DNA fragments containing the expected binding sites of the targeted TF to assess the accuracy of formaldehyde and BFPA-based ChIP-seq data sets. This will provide another metric for quantitatively evaluating data quality.

In various embodiments of the present invention, we also conceive to study (v) adapting with tagmentation. We will adapt BFPX to a new version of ChIP-seq protocol that combines chromatin immunoprecipitation with sequencing library preparation by Tn5 transposase (“tagmentation”), also known as ChIPmentation. In the initial development of ChIPmentation, it was found that tagmentation of the purified ChIP DNA leads to the same problem of data and protocol reproducibility across samples and across antibodies mentioned earlier. Part of the problem is that tagmentation was particularly sensitive to the ratio of DNA to transposase, which is highly variable and often too low to quantify in standard ChIP protocols. Moreover, purified ChIP DNA is already fragmented, and excess transposase can result in small fragments that are difficult to sequence. It turns out that performing tagmentation directly on the immunoprecipitated and bead-bound chromatin gives much improved results in terms of data quality and reproducibility, presumably because other chromatin proteins bound on beads can protect the DNA from excessive tagmentation. These observations suggest that BFPX will be a perfect match to ChIPmentation as BFPX enables the isolation of covalent protein-DNA complexes from other protein crosslinking complex backgrounds. The BFPX captured Protein-DNA complex can be anchored on streptavidin beads using azido-biotin enrichment tag and subject tagmentation by Tn5. By replacing formaldehyde, BFPX could greatly improve the performance of ChIPmentation in the study of protein-DNA interactions.

In various embodiments of the present invention, we can also compare the ChIP datasets obtained with different BFPX probes (with different DNA binding heads to avoid DNA binding bias; with different linker length to sample different DNA binding complexes). These analyses will reveal further insights into the BFPX protocol and guide its optimal application in the study of protein-DNA interactions inside cells.

### ADDITIONAL EXAMPLES

All reagents used for chemical synthesis were purchased from Sigma-Aldrich, Alfa Aesar or EMD Millipore unless otherwise specified and used without further purification. All anhydrous reactions were performed under argon or nitrogen atmosphere. All reactions, purifications, and manipulations were carried in the dark, avoiding direct exposure to natural or artificial light. Analytical thin-layer chromatography (TLC) was conducted on EMD Silica Gel 60 F254 plates with detection by ceric ammonium molybdate (CAM), anisaldehyde or UV. For flash chromatography, 60 Å silica gel (EMD) was utilized. ¹H spectra were obtained at 400, 500, or 600 MHz on a Varian spectrometers Mercury 400, VNMRS-500, or -600. Chemical shifts are recorded in ppm (δ) relative to the solvent. Coupling constants (J) are reported in Hz. ¹³C spectra were obtained at 100, 125, or 150 MHz on the same instruments. The abbreviations used for the proton spectra multiplicities are: s, singlet; b, broad; d, doublet, t, triplet; q, quartet; m, multiplet.

A Biotage Isolera Spektra FLASH system (solvent A, 0.1% TFA in water; solvent B, 0.1% TFA in acetonitrile) or an Agilent 1200 Series HPLC (solvent A: 0.1% TFA in water; solvent B: 0.1% TFA and 90% acetonitrile in water) system was used for reverse-phase high performance liquid chromatography (RP-HPLC). Mass spectra was recorded on an Agilent HPLC/Q TOF MS/MS Spectrometer.

### Example 3

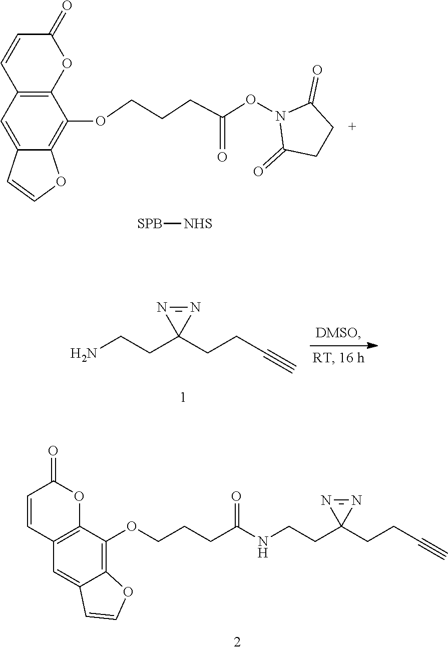

### Preparation of Compound 2 (SPB-AAD) N-(2-(3-(but-3-yn-1-yl)-3H-diazirin-3-yl)ethyl)-4-((7-oxo-7H-furo[3,2-g]chromen-9-yl)oxy)butanamide

2-(3-(but-3-yn-1-yl)-3H-diazirin-3-yl)ethan-1-amine 1 (25 mg, 0.18 mmol) was added to a solution of Succinimidyl-[4-(psoralen-8-yloxy)]butyrate SPB-NHS (50 mg, 0.13 mmol) in 0.2 mL of DMSO. The reaction was stirred at room temperature for 16 h, and after evaporation of the solvent, the crude reaction mixture was purified by reversed-phase C-18 column chromatography (0.1% TFA H₂O:ACN, 95:5 to 0:100) in 30 min to afford the compound 1 (20 mg, 38%).

¹H NMR (400 MHz, CDCl₃) δ 7.8 (dd, J=9.6, 1.2 Hz, 1H), 7.7-7.7 (m, 1H), 7.4 (s, 1H), 6.8-6.8 (m, 1H), 6.4 (d, J=1.2 Hz, 1H), 4.5-4.4 (m, 2H), 3.2 (q, J=7.0 Hz, 2H), 2.7 (t, J=7.0 Hz, 2H), 2.2-2.1 (m, 2H), 2.0-1.9 (m, 3H), 1.7-1.6 (m, 4H).

¹³C NMR (101 MHz, CDCl₃) δ 173.1, 161.0, 146.9, 144.8, 131.5, 126.2, 116.4, 114.5, 114.0, 106.9, 73.3, 69.2, 34.5, 33.0, 32.5, 31.9, 26.3, 13.2.

HRMS (ESI): calc'd. for C₂₂H₂₁N₃O₅ (M+H⁺) 408.1559, found 408.1589.

### Example 4

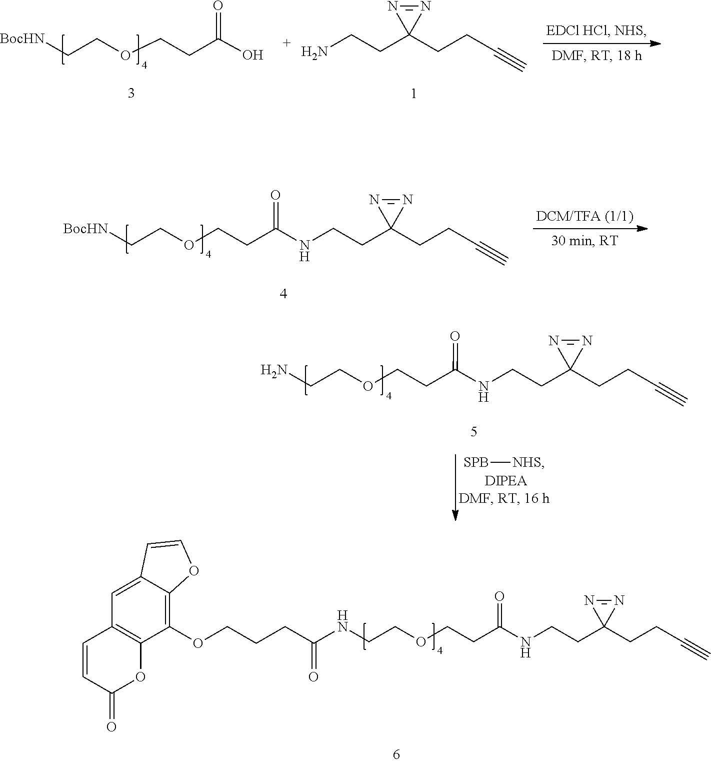

### Preparation of Compound 4 tert-butyl (18-(3-(but-3-yn-1-yl)-3H-diazirin-3-yl)-15-oxo-3,6,9,12-tetraoxa-16-azaoctadecyl)carbamate

A mixture of 2,2-dimethyl-4-oxo-3,8,11,14,17-pentaoxa-5-azaicosan-20-oic acid 3 (133 mg, 364 μmol), 1-Ethyl-3-(3-dimethylaminopropyl)carbodiimide (70 mg, 364 μmol), N-Hydroxysuccinimide (42 mg, 364 μmol) in DMF (600 μL) was stirred at room temperature for 45 min.

To this mixture was added a solution of 2-(3-(but-3-yn-1-yl)-3H-diazirin-3-yl)ethan-1-amine 1 (25 mg, 182 μmol) in DMF (300 μL). The reaction was stirred at room temperature for 18 h The reaction was stirred at room temperature for 18 h, and after evaporation of the solvent, the crude reaction mixture was purified by reversed-phase C-18 column chromatography (H₂O:MeOH, 100:0 to 0:100) in 15 column volumes (CV) to afford the compound 4 as a colorless oil (50 mg, 57%).

¹H NMR (400 MHz, CDCl₃) δ 6.7 (s, 1H), 5.1 (s, 1H), 3.7 (t, J=5.8 Hz, 2H), 3.6-3.6 (m, 11H), 3.5 (t, J=5.2 Hz, 2H), 3.3-3.2 (m, 2H), 3.1-3.0 (m, 2H), 2.4 (t, J=5.7 Hz, 2H), 2.1 (s, 1H), 2.0-1.9 (m, 3H), 1.6 (td, J=7.2, 2.3 Hz, 4H), 1.4 (s, 9H).

¹³C NMR (101 MHz, CDCl₃) δ 171.7, 155.9, 125.4, 121.7, 82.6, 70.5, 70.46, 70.4, 70.24, 70.22, 70.17, 70.14, 40.3, 36.8, 34.2, 32.5, 32.1, 28.4, 26.9, 13.2.

HRMS (ESI): calc'd. for C₂₃H₄₁N₄O₇ (M+H⁺) 485.2975, found 485.2990.

### Example 5

### Preparation of N-(2-(3-(but-3-yn-1-yl)-3H-diazirin-3-yl)ethyl)-1-(4-((7-oxo-7H-furo[3,2-g]chromen-9-yl)oxy)butanamido)-3,6,9,12-tetraoxapentadecan-15-amide (6) (SPB-PEG4-AAD)

Compound 4 (25 mg, 51 μmol) was dissolved into a solution of TFA/DCM [1:1 (v/v) 1.5 mL] and stirred for 30 min at room temperature. The reaction was concentrated under vacuum and co-evaporated with toluene three times to afford compound 5 as crude. N,N-Diisopropylethylamine (18 μL, 103 μmol) was added to a solution of the crude amine 5, SPB-NHS (25 mg, 51 μmol) in 500 μL of dry DMF. The reaction was stirred at room temperature for 16 h, then the solvent evaporated.

The reaction mixture was purified by reversed-phase C-18 column chromatography (H₂O:MeOH, 100:0 to 0:100) in 15 column volumes (CV) to afford compound 6 as a colorless oil (18 mg, 53% over 2 steps).

¹H NMR (400 MHz, CDCl₃) δ 7.8-7.8 (m, 1H), 7.7 (d, J=2.2 Hz, 1H), 7.4 (s, 1H), 6.8 (d, J=2.2 Hz, 1H), 6.7-6.7 (m, 1H), 6.4 (d, J=9.5 Hz, 1H), 4.5 (t, J=5.8 Hz, 2H), 3.7 (t, J=5.5 Hz, 2H), 3.6-3.6 (m, 11H), 3.6-3.5 (m, 2H), 3.5-3.4 (m, 2H), 3.1 (q, J=5.8 Hz, 2H), 2.6 (t, J=6.8 Hz, 2H), 2.5 (t, J=5.8 Hz, 2H), 2.2-2.1 (m, 2H), 2.0-2.0 (m, 2H), 1.7-1.6 (m, 3H), 1.3-1.2 (m, 2H).

¹³C NMR (101 MHz, CDCl₃) δ 172.7, 171.7, 160.7, 146.8, 144.6, 131.6, 126.1, 116.4, 114.6, 113.5, 106.8, 82.7, 73.2, 70.5, 70.5, 70.4, 70.3, 70.2, 70.1, 69.9, 69.4, 67.1, 39.2, 36.9, 34.2, 32.7, 32.1, 26.9, 26.1, 13.2.

HRMS (ESI): calc'd. for C₃₃H₄₃N₄O₁₀ (M+H⁺) 655.2979, found 655.2964.

### Example 6

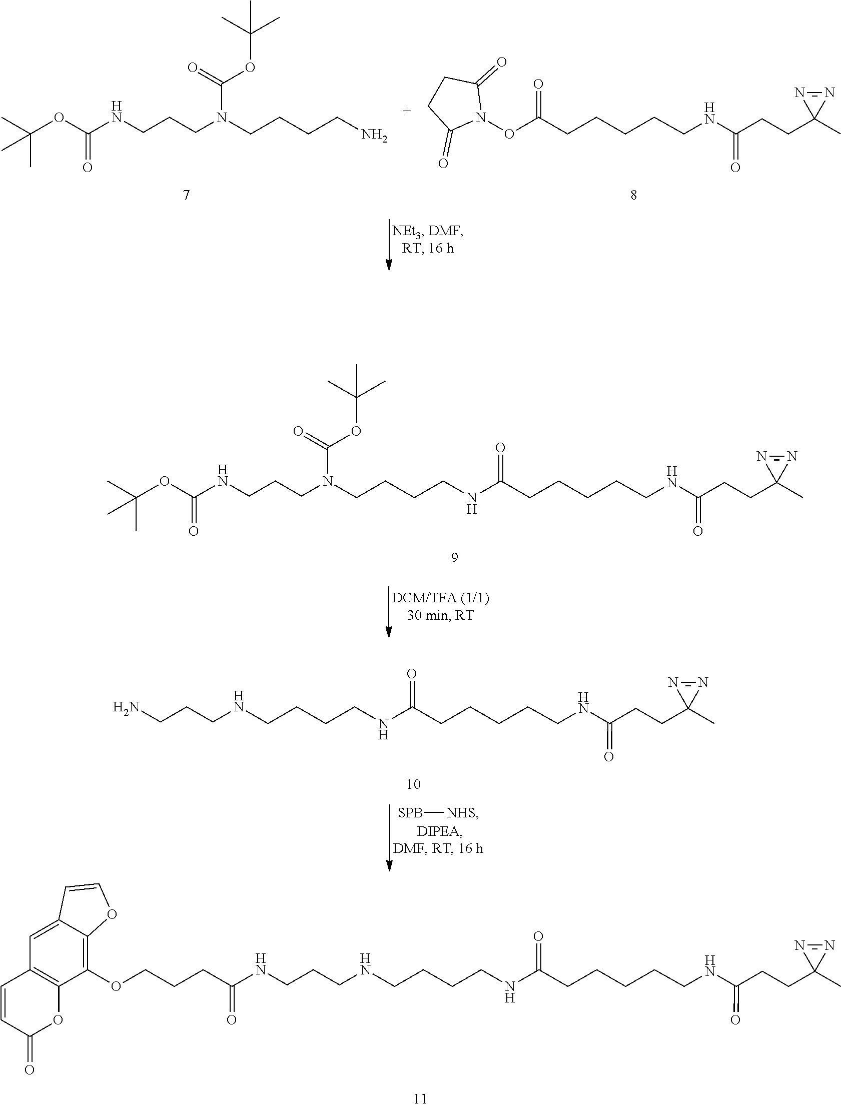

### Preparation of Compound 9

### Tert-butyl (3-((tert-butoxycarbonyl)amino)propyl)(4-(6-(3-(3-methyl-3H-diazirin-3-yl)propanamido)hexanamido)butyl)carbamate

Triethylamine (32 μL, 246 μmol) was added to a solution of 2,5-dioxopyrrolidin-1-yl 6-(3-(3-methyl-3H-diazirin-3-yl)propanamido)hexanoate (8) (40 mg, 118 μmol) and tert-butyl (4-aminobutyl)(3-((tert-butoxycarbonyl)amino)propyl)carbamate (7) (49 mg, 141 μmol) in DMF (2.1 mL).

The reaction was concentrated under vacuum and purified by reversed-phase C-18 column chromatography (H₂O:MeOH, 100:0 to 0:100) in 15 column volumes (CV) to afford compound 9 as a colorless oil (62 mg, 92%).

¹H NMR (400 MHz, CDCl₃) δ 6.8-6.5 (m, 1H), 3.6-3.3 (m, 10H), 2.4 (t, J=7.4 Hz, 2H), 2.3 (dd, J=9.0, 6.7 Hz, 2H), 2.0 (dd, J=8.8, 6.7 Hz, 2H), 2.0-1.9 (m, 4H), 1.8 (dd, J=15.0, 7.5 Hz, 7H), 1.7 (s, 9H), 1.7 (s, 9H), 1.6-1.6 (m, 2H), 1.3 (d, J=0.7 Hz, 2H).

¹³C NMR (101 MHz, CDCl₃) δ 171.8, 39.5, 36.6, 30.9, 30.4, 29.3, 28.7, 26.6, 25.8, 25.4, 20.2.

HRMS (ESI): calc'd. for C₂₈H₅₃N₆O₆(M+H⁺) 569.4027, found 569.4057.

### Example 7

### Preparation of Compound 11 (SPB-Spermidine-AD)

### 6-(3-(3-methyl-3H-diazirin-3-yl)propanamido)-N-(4-((3-(4-((7-oxo-7H-furo[3,2-g]chromen-9-yl)oxy)butanamido)propyl)amino)butyl)hexanamide

Compound 9 (62 mg, 109 μmol) was dissolved into a solution of TFA/DCM [1:1 (v/v) 1.5 mL] and stirred for 30 min at room temperature. The reaction was concentrated under vacuum and co-evaporated with toluene three times to afford compound 10 as crude.

N,N-Diisopropylethylamine (19 μL, 109 μmol) was added to a solution of the crude amine 10 and SPB-NHS (42 mg, 109 μmol) in 500 μL of dry DMF. The reaction was stirred at room temperature for 16 h, then the solvent evaporated.

The reaction mixture was purified by reversed-phase C-18 column chromatography (H₂O:MeOH, 100:0 to 0-100) in 15 column volumes (CV) to afford compound 11 as a colorless oil (18 mg, 53% over 2 steps).

¹H NMR (400 MHz, CDCl₃) δ 7.8 (dd. J=9.6, 0.8 Hz, 1H), 7.7 (dd, J=2.3, 0.8 Hz, 1H), 7.4 (s, 1H), 6.8 (dd, J=2.3, 0.8 Hz, 1H), 6.4 (dd, J=9.6, 0.8 Hz, 1H), 4.5 (t, J=5.6 Hz, 2H), 3.5 (d, J=0.8 Hz, 2H), 3.3 (q, J=6.2 Hz, 2H), 3.3-3.2 (m, 4H), 2.6 (t, J=6.5 Hz, 2H), 2.6-2.6 (m, 4H), 2.2-2.1 (m, 5H), 2.0 (dd, J=8.7, 6.8 Hz, 2H), 1.7-1.7 (m, 4H), 1.6-1.6 (m, 3H), 1.6-1.4 (m, 5H), 1.4-1.3 (m, 2H), 1.0 (d, J=0.9 Hz, 3H).

¹³C NMR (100 MHz, CDCl₃) δ 173.1, 173.0, 171.5, 161.0, 146.9, 144.9, 126.3, 116.4, 114.4, 113.7, 106.8, 73.2, 49.0, 47.3, 39.2, 39.1, 37.9, 36.3, 33.0, 30.6, 30.1, 29.0, 28.9, 27.2, 27.0, 26.2, 25.0, 19.9.

HRMS (ESI): calc'd. for C₃₃H₄₇N₆O₇ (M+H⁺) 639.3506, found 639.3602.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A compound of Formula (I): 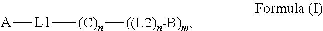
wherein:
A represents a nucleic acid-binding functional group derived from psoralen, methyltrioxsalen, benzophenone, 4′,6-diamidino-2-phenylindole (DAPI), a Hoechst dye, a polyamide, or a G quartet binding molecule, kethoxal, or a derivative thereof,
L1 is absent or represents a first linker;
C represents, when n=1, a core moiety having at least two functional groups each separately for attachment to L1 and attachment to at least one arm represented by L2-B; or C is absent when n=0;
L2 represents, when n=1, independently a second linker for each arm represented by L2-B; or L2 is absent when n=0;
B represents independently for said each arm:
a photo-reactive functional group comprising diazirine or its derivative or an aryl azide or its derivative, optionally the aryl azide or its derivative selected from phenyl azide, orthro-hydroxyphenyl azide, meta-hydroxyphenyl azide, tetrafluorophenyl azide, ortho-nitropenyl azide, meta-nitropenyl azide, or azdo-methylcoumarin; or
a detectable functional group;
wherein in at least one said each arm, B represents the photo-reactive functional group;
n=0 or 1;
m represents number of arms represented by (L2)ₙ-B, wherein m is an integer being 1 or greater when n=1, or m=1 when n=0.

2. The compound of claim 1, wherein at least one of L1 and L2 is not absent, and the at least one of L1 and L2 is cleavable.

3. The compound of claim 2, wherein L1, L2, or both independently comprise one or more of a sulfoxide-containing mass spectrometry (MS)-cleavable bond, an acid-cleavable C—S bond, a disulfide group, and an azo group.

4. The compound of claim 1, wherein n=0, m=1, and the compound is represented by Formula (II): 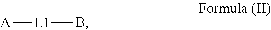
wherein L1 is absent or the first linker.

5. The compound of claim 4, wherein:
A is an amine-containing or amine-reactive derivative of the psoralen, an amine-containing or amine-reactive derivative of the methyltrioxsalen, an amine-containing or amine-reactive derivative of the benzophenone, an amine-containing or amine-reactive derivative of the 4′,6-diamidino-2-phenylindole (DAPI), an amine-containing or amine-reactive derivative of the Hoechst dye, an amine-containing or amine-reactive derivative of the polyamide, or an amine-containing or amine-reactive derivative of the G quartet binding molecule, or an amine-containing or amine-reactive derivative of kethoxal, optionally A being derived from succinimidyl-[4-(psoralen-8-yloxy)]-butyrate (SPB) or 4′-aminomethyltrioxsalen (4AMT);
B comprises a diazirine or a diazirine alkyne, optionally an amino diazirine alkyne (AAD); and
L1 is absent or the first linker, wherein the first linker comprises one or more of (i) a cleavable bond, (ii) an oligomer or polymer having a repeating unit of —OCH₂CH₂—, and (iii) an unsaturated moiety, optionally selected from a carbon-carbon double bond, a carbon-carbon triple bond, or an aryl group.

6. The compound of claim 5, wherein L1-B is derived from succinimidyl 6-(4,4′-azipentanamido)hexanoate (NHS-LC-SDA), succinimidyl 2-((4,4′-azipentanamido)ethyl)-1,3′dithiopropionate (NHS-SS-Diazirine), or 2-(3-(But-3-yn-1-yl)-3H-diazirin-3-yl)ethan-1-amine (AAD); and/or wherein A is derived from 4′-aminomethyltrioxsalen (4AMT) or succinimidyl-[4-(psoralen-8-yloxy)]-butyrate (SPB); and wherein optionally the photocrosslinking molecule is represented by Formula (IIa) or Formula (IIc):

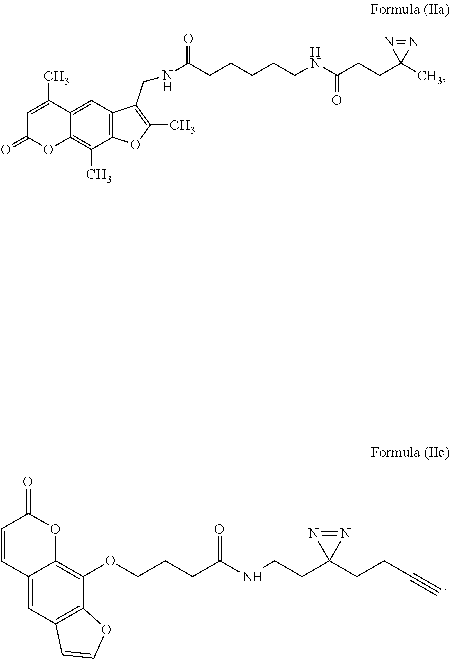 [SMILES: Cc1oc2c(C)c3oc(=O)cc(C)c3cc2c1CNC(=O)CCCCCNC(=O)CCC1(C)N=N1 ; C#CCCC1(CCNC(=O)CCCOc2c3occc3cc3ccc(=O)oc23)N=N1]

7. The compound of claim 5, wherein:
A is derived from succinimidyl-[4-(psoralen-8-yloxy)]-butyrate (SPB) or 4′-aminomethyltrioxsalen (4AMT);
B comprises a diazirine or a diazirine alkyne, optionally an amino diazirine alkyne (AAD); and
L1 is the first linker comprising one or more of (i) a cleavable bond, (ii) an oligomer or polymer having a repeating unit of —OCH₂CH₂—, and/or (iii) an unsaturated moiety, said unsaturated moiety optionally selected from a carbon-carbon double bond or an aryl group; and
wherein optionally the photocrosslinking molecule is represented by Formula (IIb), Formula (IId), Formula (IIe), or Formula (IIf):

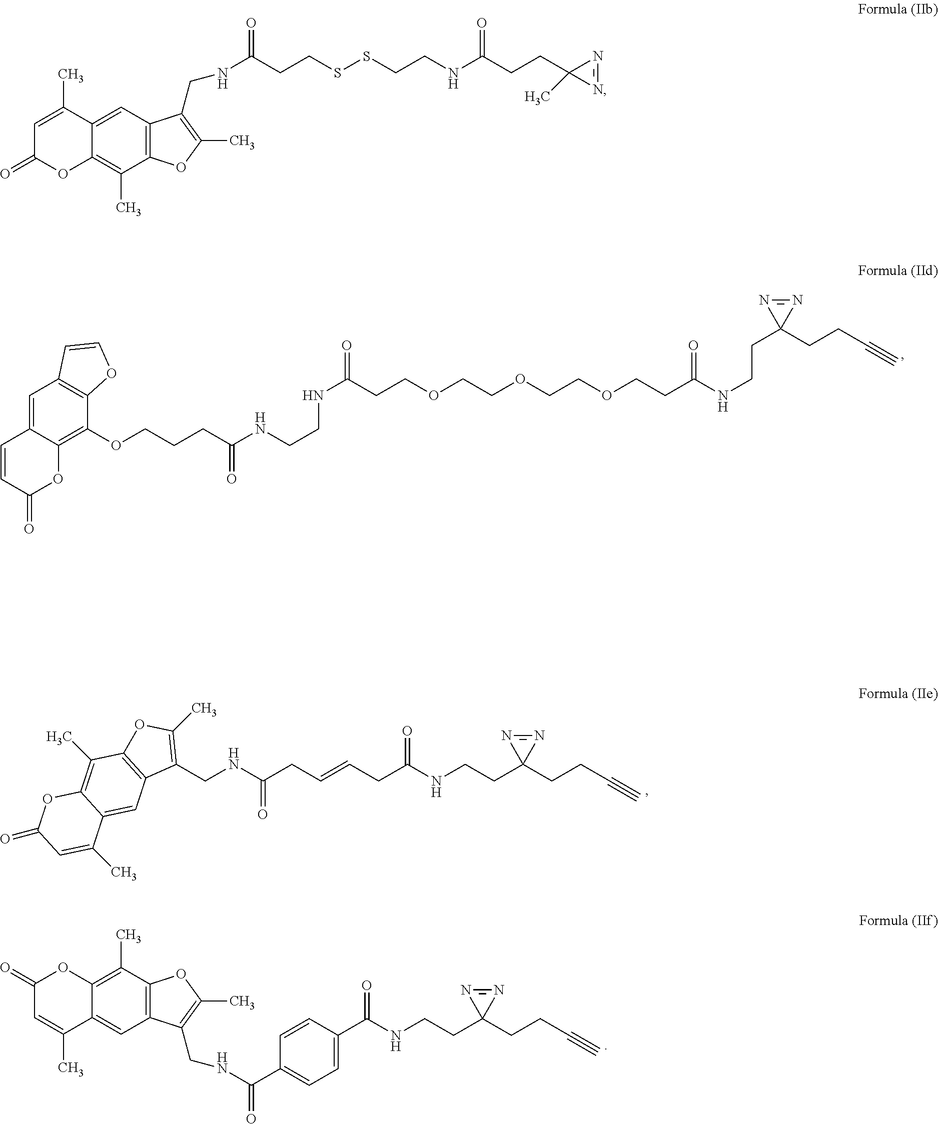 [SMILES: Cc1oc2c(C)c3oc(=O)cc(C)c3cc2c1CNC(=O)CCSSCCNC(=O)CCC1(C)N=N1 ; C#CCCC1(CCNC(=O)CCOCCOCCOCCC(=O)NCCNC(=O)CCCOc2c3occc3cc3ccc(=O)oc23)N=N1 ; C#CCCC1(CCNC(=O)C/C=C/CC(=O)NCc2c(C)oc3c(C)c4oc(=O)cc(C)c4cc23)N=N1 ; C#CCCC1(CCNC(=O)c2ccc(C(=O)NCc3c(C)oc4c(C)c5oc(=O)cc(C)c5cc34)cc2)N=N1]

8. The compound of claim 4, wherein:
A is selected from the group consisting of: 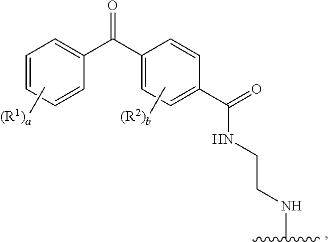
wherein:
R¹ is independently H, halo, OH, optionally substituted alkoxy, or optionally substituted alkyl;
R² is independently H, halo, OH, optionally substituted alkoxy, or optionally substituted alkyl;
a is 0, 1, 2, 3, 4, or 5; and
b is 0, 1, 2, 3, or 4; 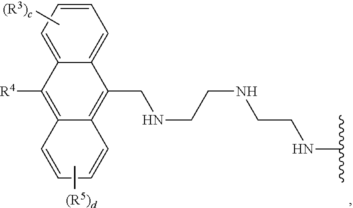
wherein:
R³ is independently H, halo, OH, optionally substituted alkoxy, or optionally substituted alkyl;
R⁴ is H, halo, OH, optionally substituted alkoxy, or optionally substituted alkyl;
R⁵ is independently H, halo, OH, optionally substituted alkoxy, or optionally substituted alkyl;
c is 0, 1, 2, 3, or 4; and
d is 0, 1, 2, 3, or 4; 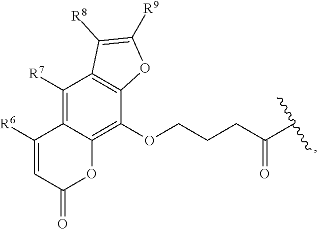
wherein:
R⁶ is H, halo, OH, optionally substituted alkoxy, or optionally substituted alkyl;
R⁷ is H, halo, OH, optionally substituted alkoxy, or optionally substituted alkyl;
R⁸ is H, halo, OH, optionally substituted alkoxy, or optionally substituted alkyl; and
R⁹ is H, halo, OH, optionally substituted alkoxy, or optionally substituted alkyl; 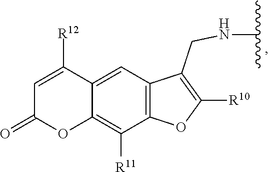
wherein:
R¹⁰ is H, halo, OH, optionally substituted alkoxy, or optionally substituted alkyl;
R¹¹ is H, halo, OH, optionally substituted alkoxy, or optionally substituted alkyl; and
R¹² is H, halo, OH, optionally substituted alkoxy, or optionally substituted alkyl; 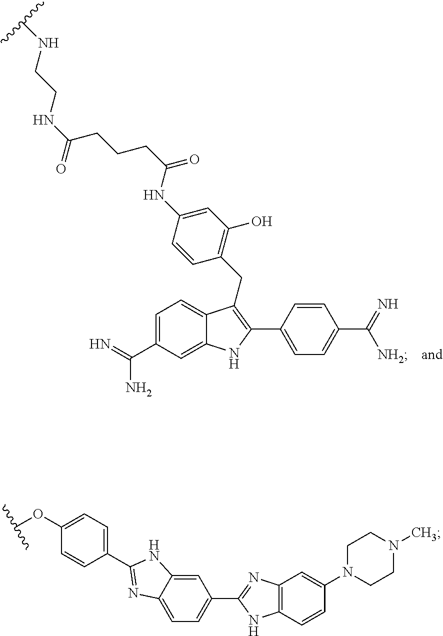 [SMILES: *NCCNC(=O)CCCC(=O)Nc1ccc(Cc2c(-c3ccc(C(=N)N)cc3)[nH]c3cc(C(=N)N)ccc23)c(O)c1 ; *Oc1ccc(-c2nc3ccc(-c4nc5cc(N6CCN(C)CC6)ccc5[nH]4)cc3[nH]2)cc1]
L1 is absent or L is selected from the group consisting of: 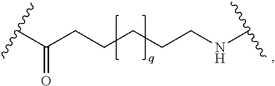
wherein:
q is 0, 1, 2, 3, or 4; 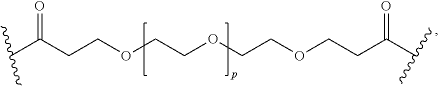
wherein:
p is 0, 1, 2, 3, or 4; 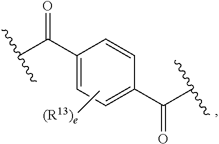
wherein:
R¹³ is independently H, halo, OH, optionally substituted alkoxy, or optionally substituted alkyl;
and e is 0, 1, 2, 3 or 4; 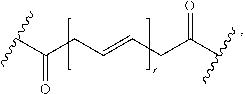
wherein:
r is 0, 1, 2, 3, or 4; 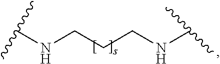
wherein:
s is 0, 1, 2, 3, or 4; 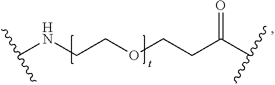
wherein:
t is 0, 1, 2, 3 or 4; 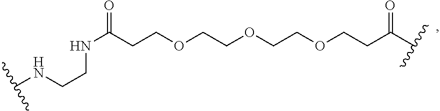 [SMILES: *NCCNC(=O)CCOCCOCCOCCC(*)=O]
u is 0, 1, 2, 3, or 4; 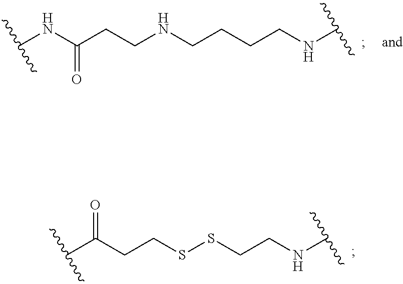 [SMILES: *NCCCCNCCC(=O)N* ; *NCCSSCCC(*)=O]
and
B is selected from the group consisting of:

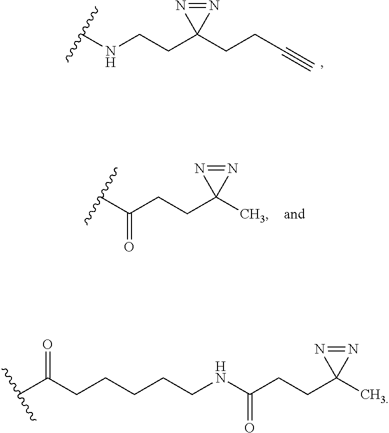 [SMILES: *NCCC1(CCC#C)N=N1 ; *C(=O)CCC1(C)N=N1 ; *C(=O)CCCCCNC(=O)CCC1(C)N=N1]

9. The compound of claim 4, wherein:
A is selected from the group consisting of: 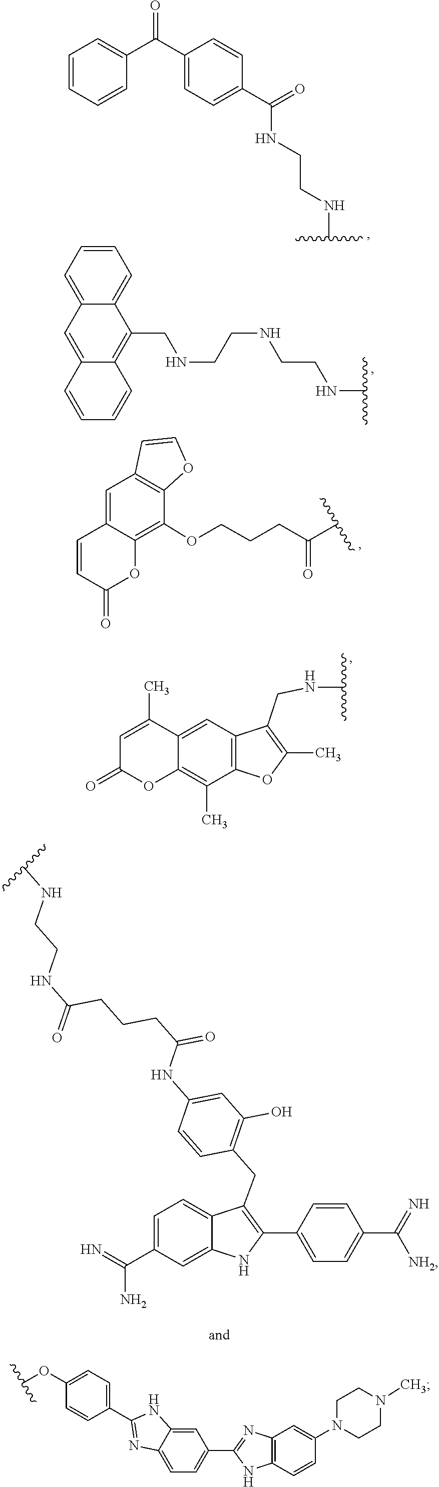
L1 is absent or L1 is selected from the group consisting of: 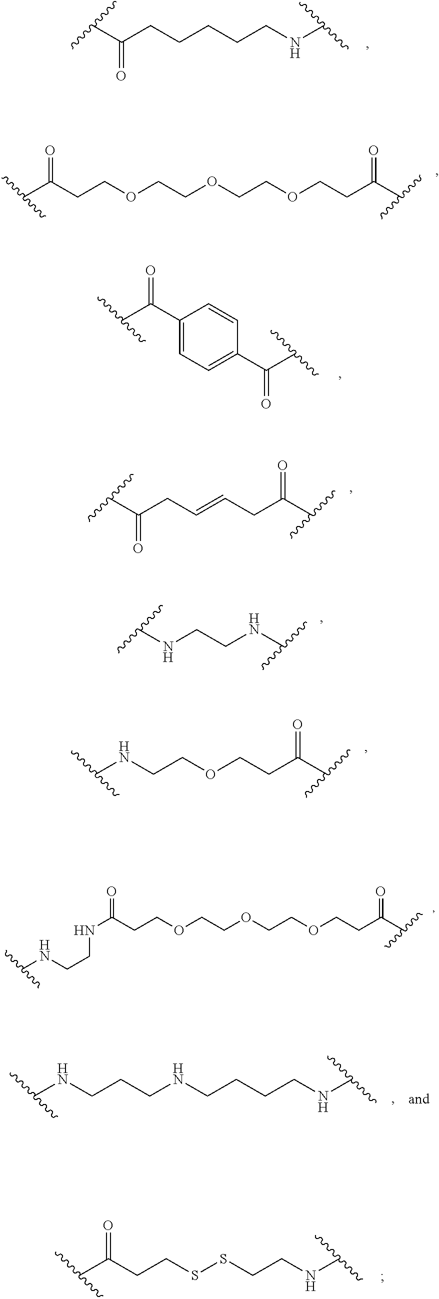
and
B is selected from the group consisting of:

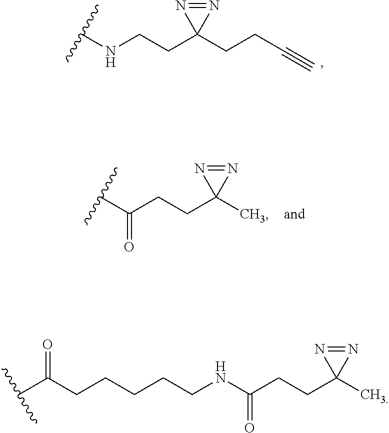 [SMILES: *NCCC1(CCC#C)N=N1 ; *C(=O)CCC1(C)N=N1 ; *C(=O)CCCCCNC(=O)CCC1(C)N=N1]

10. The compound of claim 4, wherein the compound is:

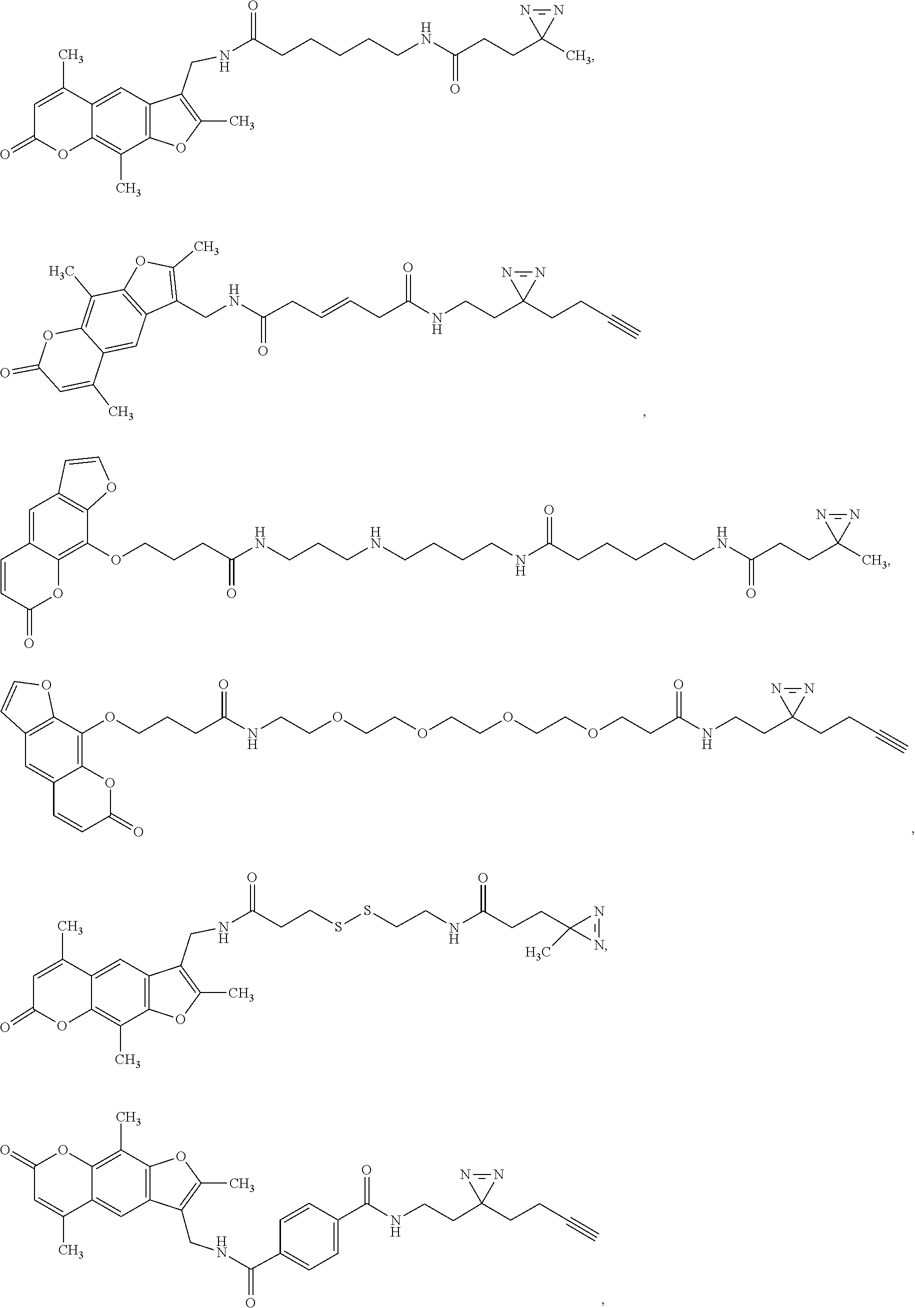

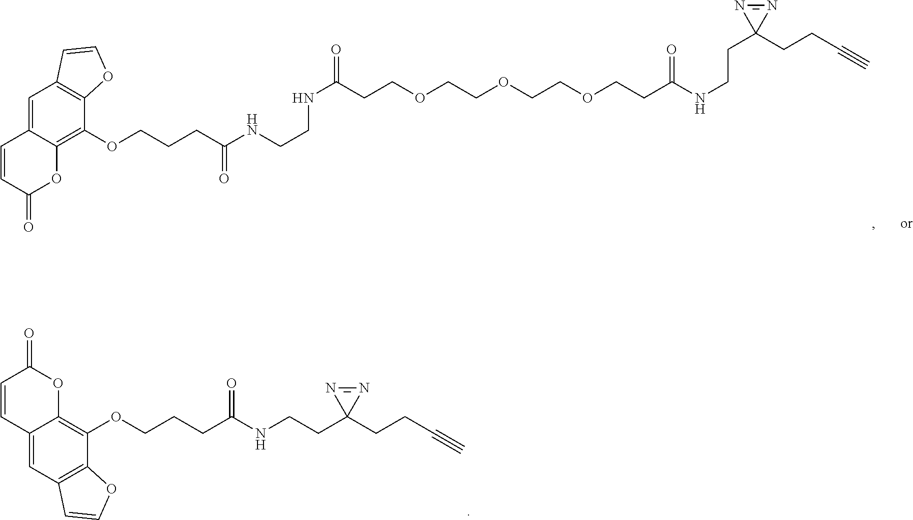 [SMILES: C#CCCC1(CCNC(=O)CCOCCOCCOCCC(=O)NCCNC(=O)CCCOc2c3occc3cc3ccc(=O)oc23)N=N1 ; C#CCCC1(CCNC(=O)CCCOc2c3occc3cc3ccc(=O)oc23)N=N1]

11. The compound of claim 5, wherein L1 comprises 2 to 20 carbons or 20-100 carbons in length.

12. The compound of claim 1, wherein n=1, m is an integer being 2 or greater, and C represents a core moiety having at least three functional groups each separately for attachment to L1 and attachment to the at least two arms each represented by (L2-B), so that the compound is represented by Formula (III):

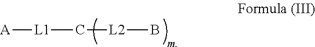

13. The compound of claim 12, wherein B comprises diazirine or an azide diazirine in one of the at least two arms, and B represents a detectable functional group in another one of the at least two arms, said detectable function group comprising a fluorophore, a biotin, a chromophore, a chromogen, a quantum dot, a fluorescent microsphere, or a nanoparticle.

14. The compound of claim 12, wherein L1, L2, or both independently comprise one or more of (i) a cleavable bond, (ii) an oligomer or polymer having a repeating unit of —OCH₂CH₂—, and (iii) an unsaturated moiety.

15. The compound of claim 12, wherein C represents a dendritic core moiety comprising at least three surface functional groups each separately for attachment to L1 and attachment to the at least two arms each represented by L2-B.

16. (canceled)

17. A method of crosslinking a nucleic acid with a protein in a system, comprising:
providing a compound of claim 1;
providing a system, wherein the system comprises a nucleic acid and a protein;
contacting the compound with the system; and
irradiating the system and the compound with an ultraviolet light under conditions effective to crosslink the nucleic acid with the protein.

18. (canceled)

19. (canceled)

20. The method of claim 17, further comprising performing one or more of immuno precipitation, chromatic precipitation, 3D chromatin conformation capture, mass spectrometry, and electrophoresis, with the system.

21. The method of claim 17, wherein element L1, L2, or both of the compound is independently cleavable, and the method further comprises adding a cleaving agent to the system to cleave the elements L1, L2, or both; or wherein element A of the compound is derived from psoralen, and the method further comprises applying an ultraviolet light of about 230 nm in wavelength to cleave the element A; thereby generating a fingerprint of crosslinked proteins in proximity to nucleic acids in the system.

22. A method for preparing the compound of claim 12, comprising:
providing an azide derivative of a nucleic acid-binding, photo-reactive agent comprising psoralen, methyltrioxsalen, benzophenone, 4′,6-diamidino-2-phenylindole (DAPI), a Hoechst dye, a polyamide, or a G quartet binding molecule, kethoxal, or a derivative thereof;
providing an azide derivative of a photo-reactive agent that comprises a diazirine moiety so as to obtain an azide-diazirine bifunctional, photo-reactive agent, and said photo-reactive agent optionally further comprising an alkyne group, or providing an aryl azide, said aryl azide optionally selected from phenyl azide, orthro-hydroxyphenyl azide, meta-hydroxyphenyl azide, tetrafluorophenyl azide, ortho-nitropenyl azide, meta-nitropenyl azide or azdo-methylcoumarin;
optionally providing an azide derivative of a detectable agent comprising a fluorophore, a biotin, a chromophore, a chromogen, a quantum dot, a fluorescent microsphere, or a nanoparticle;
providing a multi-arm agent having at least three functional groups each independently comprising an alkyne; and
combining each azide derivatives and, if provided, the aryl azide, together with the multi-arm agent in one reaction vessel to prepare the compound.

23. (canceled)

24. The method of claim 22, wherein the nucleic acid-binding, photo-reactive agent comprises a first primary amine functional group, and providing the azide derivative of the nucleic acid-binding, photo-reactive agent comprises converting the first primary amine functional group to a first azide-containing moiety, optionally via reacting the nucleic acid-binding, photo-reactive agent with imidazole-1-sulfonyl azide; and/or wherein the photo-reactive agent that comprises a diazirine moiety further comprises a second primary amine functional group or is modified with the second primary amino functional group, and providing the azide derivative of said photo-reactive agent comprises converting the second primary amine functional group to a second azide-containing moiety, optionally via reacting said photo-reactive agent with imidazole-1-sulfonyl azide.
